SEQUENCE LISTING (130 sequences)

SEQ ID NO: 1 - protein, 5030 aa, Homo sapiens [sequence omitted]

SEQ ID NO: 2 - DNA, 921 bp, synthetic construct
  misc_feature 1..921: note = N-terminal fragment of MUC5AC
  source 1..921: mol_type = other DNA
accacccattctcaacctgttactcgtgattgtcatctgcgttgcacctggactaaatggttcgacgttgacttcccgtccccaggtccacacggtggtgacaaggaaacctataacaacatcattcgttccggtgagaaaatctgccgtcgtccggaggaaatcacccgtctgcagtgccgtgcagagtcccacccggaggtatctatcgaacatctgggccaggttgtgcagtgcagccgtgaagaaggtctggtttgccgtaaccaagatcagcagggcccgttcaaaatgtgcctgaactatgaagtccgtgtcctgtgctgcgaaaccccaaaaggctgtccagttacttctaccccggttaccgcgccgtccactccaagcggccgcgcgaccagcccgacccagagcacctcctcttggcagaaatcccgcaccactaccctggttactacctctactacctccactccacagacttccaccacctccgccccgactaccagcactaccagcgccccgaccactagcactacctccgctccgaccacctccaccacttctaccccgcagacctctatctcttctgcgccgaccagctctaccaccagcgctccgactagctccacgatttctgctcgtactacttctatcatttccgcccctacgacctctaccacttctagccctaccacctctaccacgtccgcgaccaccacctccactacctctgcaccaacttcctctactacgagcacgccgcagacttctaaaacctctgcggcaacctcttctaccaccagcagctctggcaccactccgagcccggtgaccaccactagcaccgcttctgtgtccaagaccagcacctctcacgtgtctgtttctaaaacgacccactcccagccggttacccgc

SEQ ID NO: 3 - protein, 306 aa, synthetic construct
  REGION 1..306: note = N-terminal fragment of MUC5AC
TTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTTSAPTTSTTSAPTTSTTSTPQTSISSAPTSSTTSAPTSSTISARTTSIISAPTTSTTSSPTTSTTSATTTSTTSAPTSSTTSTPQTSKTSAATSSTTSSSGTTPSPVTTTSTASVSKTTSHVSVSKTTHSQPVTR

SEQ ID NO: 4 - DNA, 384 bp, synthetic construct
  misc_feature 1..384: note = N-terminal fragment of MUC5AC
  source 1..384: mol_type = other DNA
actactcattctcaacctgtaactcgtgattgtcatctgcgctgtacttggactaaatggtttgacgtggacttcccgtcccctggcccgcacggtggtgataaagaaacctacaataacatcattcgctctggtgagaaaatctgccgtcgtccggaagaaatcactcgtctgcaatgtcgtgccgaatcccacccggaggtgagcatcgaacacctgggtcaggttgttcagtgttctcgtgaggaaggtctggtatgccgtaaccaagatcagcaaggcccattcaaaatgtgcctgaactacgaagttcgtgttctgtgttgcgagactccgaaaggttgcccggttacgagcacgcctgtcaccgcaccgagcacgccg

SEQ ID NO: 5 - protein, 128 aa, synthetic construct
  REGION 1..128: note = N-terminal fragment of MUC5AC
TTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTP

SEQ ID NO: 6 - protein, 45 aa, synthetic construct
  REGION 1..45: note = MUC5AC termolysin fragment2
LVTTSTTSTPQTSTTSAPTTSTTSAPTTSTTSAPTTSTTSTPQTS

SEQ ID NO: 7 - protein, 46 aa, synthetic construct
  REGION 1..46: note = MUC5AC thermolysin fragment4
SSPTTSTTPTPQTSTTSSPTTSTTSAPTTSTTSAPTTSTTSTPQTS

SEQ ID NO: 8 - protein, 59 aa, synthetic construct
  REGION 1..59: note = MUC5AC thermolysin fragment1
ITSAPTTSTTSAPTTSTTSAPTTSTTSAPTTSTTSAPTTSTTSTPTSSTTSTPQTSTTS

SEQ ID NO: 9 - protein, 54 aa, synthetic construct
  REGION 1..54: note = MUC5AC thermolysin fragment3
ASIPSTTSAPTTSTTSAPTTSTTSAPTTSTTSTPQTTTSSAPTSSTTSAPTTST

SEQ ID NO: 10 - protein, 45 aa, synthetic construct
  REGION 1..45: note = MUC5AC thermolysin fragment5
MTSGPGTTPSPVPTTSTTSAPTTSTTSGPGTTPSPVPTTSTTSAP

SEQ ID NO: 11 - protein, 54 aa, synthetic construct
  REGION 1..54: note = MUC5AC thermolysin fragment6
ITSMPSGPGTTPSPVPTTSTTSAPTTSTTSGPGTTPSPVPTTSTTSAPTTSTTS

SEQ ID NO: 12 - protein, 59 aa, synthetic construct
  REGION 1..59: note = MUC5AC thermolysin fragment7
LSPVPTTSTTSAPTTSTTSGPGTTPSPVPTTSTTSAPTTSTTSGPGTTPSPVPTTSTTP

SEQ ID NO: 13 - protein, 85 aa, synthetic construct
  REGION 1..85: note = MUC5AC truncation construct (85)
ATMSVGRRKLALLWALALALACTTTHSQPVTRDCHLRCTWTKWFDVDFPSPGPHGGDKETYNNIIRSGEKICRRPEEITRLQCRA

SEQ ID NO: 14 - protein, 136 aa, synthetic construct
  REGION 1..136: note = MUC5AC truncation construct (136)
ATMSVGRRKLALLWALALALACTTTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPK

SEQ ID NO: 15 - protein, 151 aa, synthetic construct
  REGION 1..151: note = MUC5AC truncation construct (151)
ATMSVGRRKLALLWALALALACTTTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTP

SEQ ID NO: 16 - protein, 187 aa, synthetic construct
  REGION 1..187: note = MUC5AC truncation construct (187)
ATMSVGRRKLALLWALALALACTTTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTT

SEQ ID NO: 17 - protein, 289 aa, synthetic construct
  REGION 1..289: note = MUC5AC truncation construct (289)
ATMSVGRRKLALLWALALALACTTTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTTSAPTTSTTSAPTTSTTSTPQTSISSAPTSSTTSAPTSSTISARTTSIISAPTTSTTSSPTTSTTSATTTSTTSAPTSSTTSTPQTSKTSAATSS

SEQ ID NO: 18 - protein, 338 aa, synthetic construct
  REGION 1..338: note = MUC5AC truncation construct (338)
ATMSVGRRKLALLWALALALACTTTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTTSAPTTSTTSAPTTSTTSTPQTSISSAPTSSTTSAPTSSTISARTTSIISAPTTSTTSSPTTSTTSATTTSTTSAPTSSTTSTPQTSKTSAATSSTTSSSGTTPSPVTTTSTASVSKTSTSHVSVSKTTHSQPVTRCTHHHHHH

SEQ ID NO: 19 - protein, 502 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 20 - DNA, 3600 bp, Homo sapiens
  source 1..3600: mol_type = unassigned DNA
gaagagactcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacaaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtatgacagaatacatttgaaaacattggttatattaccaagactttgactagaatgtcgtatttgaggatataaacccataggtaataaacccacaggtactacaaacaaagtctgaagtcagccttggtttggcttcctagtgtcaattaaacttctaaaagtttaatctgagattccttataaaaacttccagcaaagcaactttaaaaaagtctgtgtgggccgggcgcggtggctcacgcctgtaatcccagcactttgatccgccgaggcgggcggatcacgaggtcaggagatccagaccatcctggctaacacagtgaaaccccgtctctactaaaaatacaaaaaaagttagccgggcgtggtggtgggggcctgtagtcccagctactcaggaggctgaggcaggagaacggcatgaacccgggaggcagggcttgcagtgagccaagatcatgccgctgcactccagcctgggagacaaagtgagactccgtcaaaaaaaaaaaaaagtctatgtggtcagtcactactcttgctgcagttatgaaaagaatgaggccaagtctgatgaaaataaacttattttgaaaaca

SEQ ID NO: 21 - protein, 344 aa, Homo sapiens
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 22 - protein, 2631 aa, Homo sapiens
GACCCTGGGAAATGCTTCTATCCCTGAGAGGAGGCTCAGCACAGAAGGAGGAAGGACAGCAGGGCCAACAGTCACAGCAGCCCTGACCAGAGCATTCCTGGAGCTCAAGCTCCTCTACAAAGAGGTGGACAGAGAAGACAGCAGAGACCATGGGACCCCCCTCAGCCCCTCCCTGCAGATTGCATGTCCCCTGGAAGGAGGTCCTGCTCACAGCCTCACTTCTAACCTTCTGGAACCCACCCACCACTGCCAAGCTCACTATTGAATCCACGCCGTTCAATGTCGCAGAGGGGAAGGAGGTTCTTCTACTCGCCCACAACCTGCCCCAGAATCGTATTGGTTACAGCTGGTACAAAGGCGAAAGAGTGGATGGCAACAGTCTAATTGTAGGATATGTAATAGGAACTCAACAAGCTACCCCAGGGCCCGCATACAGTGGTCGAGAGACAATATACCCCAATGCATCCCTGCTGATCCAGAACGTCACCCAGAATGACACAGGATTCTATACCCTACAAGTCATAAAGTCAGATCTTGTGAATGAAGAAGCAACCGGACAGTTCCATGTATACCCGGAGCTGCCCAAGCCCTCCATCTCCAGCAACAACTCCAACCCCGTGGAGGACAAGGATGCTGTGGCCTTCACCTGTGAACCTGAGGTTCAGAACACAACCTACCTGTGGTGGGTAAATGGTCAGAGCCTCCCGGTCAGTCCCAGGCTGCAGCTGTCCAATGGCAACATGACCCTCACTCTACTCAGCGTCAAAAGGAACGATGCAGGATCCTATGAATGTGAAATACAGAACCCAGCGAGTGCCAACCGCAGTGACCCAGTCACCCTGAATGTCCTCTATGGCCCAGATGGCCCCACCATTTCCCCCTCAAAGGCCAATTACCGTCCAGGGGAAAATCTGAACCTCTCCTGCCACGCAGCCTCTAACCCACCTGCACAGTACTCTTGGTTTATCAATGGGACGTTCCAGCAATCCACACAAGAGCTCTTTATCCCCAACATCACTGTGAATAATAGCGGATCCTATATGTGCCAAGCCCATAACTCAGCCACTGGCCTCAATAGGACCACAGTCACGATGATCACAGTCTCTGGAAGTGCTCCTGTCCTCTCAGCTGTGGCCACCGTCGGCATCACGATTGGAGTGCTGGCCAGGGTGGCTCTGATATAGCAGCCCTGGTGTATTTTCGATATTTCAGGAAGACTGGCAGATTGGACCAGACCCTGAATTCTTCTAGCTCCTCCAATCCCATTTTATCCCATGGAACCACTAAAAACAAGGTCTGCTCTGCTCCTGAAGCCCTATATGCTGGAGATGGACAACTCAATGAAAATTTAAAGGGAAAACCCTCAGGCCTGAGGTGTGTGCCACTCAGAGACTTCACCTAACTAGAGACAGGCAAACTGCAAACCATGGTGAGAAATTGACGACTTCACACTATGGACAGCTTTTCCCAAGATGTCAAAACAAGACTCCTCATCATGATAAGGCTCTTACCCCCTTTTAATTTGTCCTTGCTTATGCCTGCCTCTTTCGCTTGGCAGGATGATGCTGTCATTAGTATTTCACAAGAAGTAGCTTCAGAGGGTAACTTAACAGAGTATCAGATCTATCTTGTCAATCCCAACGTTTTACATAAAATAAGAGATCCTTTAGTGCACCCAGTGACTGACATTAGCAGCATCTTTAACACAGCCGTGTGTTCAAATGTACAGTGGTCCTTTTCAGAGTTGGACTTCTAGACTCACCTGTTCTCACTCCCTGTTTTAATTCAACCCAGCCATGCAATGCCAAATAATAGAATTGCTCCCTACCAGCTGAACAGGGAGGAGTCTGTGCAGTTTCTGACACTTGTTGTTGAACATGGCTAAATACAATGGGTATCGCTGAGACTAAGTTGTAGAAATTAACAAATGTGCTGCTTGGTTAAAATGGCTACACTCATCTGACTCATTCTTTATTCTATTTTAGTTGGTTTGTATCTTGCCTAAGGTGCGTAGTCCAACTCTTGGTATTACCCTCCTAATAGTCATACTAGTAGTCATACTCCCTGGTGTAGTGTATTCTCTAAAAGCTTTAAATGTCTGCATGCAGCCAGCCATCAAATAGTGAATGGTCTCTCTTTGGCTGGAATTACAAAACTCAGAGAAATGTGTCATCAGGAGAACATCATAACCCATGAAGGATAAAAGCCCCAAATGGTGGTAACTGATAATAGCACTAATGCTTTAAGATTTGGTCACACTCTCACCTAGGTGAGCGCATTGAGCCAGTGGTGCTAAATGCTACATACTCCAACTGAAATGTTAAGGAAGAAGATAGATCCAATTAAAAAAAATTAAAACCAATTTAAAAAAAAAAAGAACACAGGAGATTCCAGTCTACTTGAGTTAGCATAATACAGAAGTCCCCTCTACTTTAACTTTTACAAAAAAGTAACCTGAACTAATCTGATGTTAACCAATGTATTTATTTCTGTGGTTCTGTTTCCTTGTTCCAATTTGACAAAACCCACTGTTCTTGTATTGTATTGCCCAGGGGGAGCTATCACTGTACTTGTAGAGTGGTGCTGCTTTAATTCATAAATCACAAATAAAAGCCAATTAGCTCTATAACT

SEQ ID NO: 23 - protein, 129 aa, synthetic construct
  REGION 1..129: note = CEACAM6 truncation construct (residues 191-320)
LQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVS

SEQ ID NO: 24 - protein, 130 aa, synthetic construct
  REGION 1..130: note = CEACAM5 truncation construct (residues 191-321)
LQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYA

SEQ ID NO: 25 - protein, 130 aa, synthetic construct
  REGION 1..130: note = CEACAM8 truncation construct (residues 191-321)
LQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSD

SEQ ID NO: 26 - protein, 130 aa, synthetic construct
  REGION 1..130: note = CEACAM1 truncation construct (residues 191-321)
LQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTE

SEQ ID NO: 27 - protein, 8 aa, synthetic construct
  REGION 1..8: note = MUC5AC tandem repeat
TTSTTSAP

SEQ ID NO: 28 - protein, 8 aa, synthetic construct
  REGION 1..8: note = MUC5AC tandem repeat consensus peptide
GSTPSPVP

SEQ ID NO: 29 - protein, 8 aa, synthetic construct
  REGION 1..8: note = MUC5AC tandem repeat consensus peptide
TASTTSGP

SEQ ID NO: 30 - protein, 16 aa, synthetic construct
  REGION 1..16: note = MUC5AC O-glycosylation motiff
GTTPSPVPTTSTTSAP

SEQ ID NO: 31 - protein, 16 aa, synthetic construct
  REGION 1..16: note = MUC5AC O-glycosylation motiff
GTTPSAVPTTSTTSVP

SEQ ID NO: 32 - protein, 16 aa, synthetic construct
  REGION 1..16: note = MUC5AC O-glycosylation motiff
GTTPSPVPTTSITSVP

SEQ ID NO: 33 - protein, 11 aa, synthetic construct
  REGION 1..11: note = MUC5AC tandem repeat
TTSTTSAPTTS

SEQ ID NO: 34 - protein, 307 aa, synthetic construct
  REGION 1..307: note = NPC-1 antigen (variant 1)
TTHSQPVTRDCHLRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTTSAPTTSTTSAPTTSTTSTPQTSISSAPTSSTTSAPTSSTISARTTSIISAPTTSTTSSPTTSTTSATTTSTTSAPTSSTTSTPQTSKTSAATSSTTSSSGTTPSPVTTTSTASVSKTSTSHVSVSKTTHSQPVTR

SEQ ID NO: 35 - protein, 281 aa, synthetic construct
  REGION 1..281: note = NPC-1 antigen (variant 2)
GTHTTPVTRNCHPRCTWTKWFDVDFPSPGPHGGDKETYNNIIRSGEKICRRPEEITRLQCRAKSHPEVSIEHLGQVVQCSREEGLVCRNQDQQGPFKMCLNYEVRVLCCETPRGCPVTSTPVTAPSTPSGRAISPTQSTSSWQKSRTTTLVTTSTTSTPQTSTTYAHTTSTTSAPTARTTSAPTTRTTSASPASTTSGPGNTPSPVPTTSTISAPTTSITSAPTTSTTSAPTSSTTSAPTTSTTSAPTSSTTSSPQTSTTSAPTTSTTSGPGTTPSPVPTT

SEQ ID NO: 36 - protein, 16 aa, synthetic construct
  REGION 1..16: note = MUC5AC NPC-1 epitope (1)
TTSTTSAPTTSTTSAP

SEQ ID NO: 37 - protein, 15 aa, synthetic construct
  REGION 1..15: note = MUC5AC NPC-1 epitope (2)
GCPVTSTPVTAPSTP

SEQ ID NO: 38 - protein, 424 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQNALPQENGLSPGAIAGIVIGVVALVALIAVALACFLHFGKTGSSGPLQ

SEQ ID NO: 39 - protein, 349 aa, Homo sapiens
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 40 - protein, 92 aa, synthetic construct
  REGION 1..92: note = CEACAM5 16C3 epitope
  VAR_SEQ 8..67: note = MISC_FEATURE - Xaa can be any amino acid
GPDAPTIXXXXXXXXXXXXXXXXXXXXXXXXXXXXXXXXXXXXXXXXXXXXXXXXXXXXXXXXXXXXGSYTCQAHNSDTGLNRTTVTTITVY

SEQ ID NO: 41 - protein, 7 aa, synthetic construct
  REGION 1..7: note = CEACAM 16C3 epitope N-terminal flanking peptide
GPDAPTI

SEQ ID NO: 42 - protein, 25 aa, synthetic construct
  REGION 1..25: note = CEACAM5 16C3 C-terminal flanking peptide
GSYTCQAHNSDTGLNRTTVTTITVY

SEQ ID NO: 43 - protein, 92 aa, synthetic construct
  REGION 1..92: note = CEACAM6 16C3 epitope
  VAR_SEQ 8..67: note = MISC_FEATURE - Xaa can be any amino acid
GPDGPTIXXXXXXXXXXXXXXXXXXXXXXXXXXXXXXXXXXXXXXXXXXXXXXXXXXXXXXXXXXXXGSYMCQAHNSATGLNRTTVTMITVS

SEQ ID NO: 44 - protein, 25 aa, synthetic construct
  REGION 1..25: note = CEACAM6 C-terminal flanking peptide
GSYMCQAHNSATGLNRTTVTMITVS

SEQ ID NO: 45 - protein, 319 aa, Homo sapiens
MVGKMWPVLWTLCAVRVTVDAISVETPQDVLRASQGKSVTLPCTYHTSTSSREGLIQWDKLLLTHTERVVIWPFSNKNYIHGELYKNRVSISNNAEQSDASITIDQLTMADNGTYECSVSLMSDLEGNTKSRVRLLVLVPPSKPECGIEGETIIGNNIQLTCQSKEGSPTPQYSWKRYNILNQEQPLAQPASGQPVSLKNISTDTSGYYICTSSNEEGTQFCNITVAVRSPSMNVALYVGIAVGVVAALIIIGIIIYCCCCRGKDDNTEDKEDARPNREAYEEPPEQLRELSREREEEDDYRQEEQRSTGRESPDHLDQ

SEQ ID NO: 46 - protein, 56 aa, synthetic construct
  REGION 1..56: note = A33 antigen mass spec fragment 1
ASQGKSVTLPCTYHTSTSSREGLIQWDKLLLTHTERVVIWPFSNKNYIHGELYKNR

SEQ ID NO: 47 - protein, 67 aa, synthetic construct
  REGION 1..67: note = A33 antigen mass spec fragment 2
VRLLVLVPPSKPECGIEGETIIGNNIQLTCQSKEGSPTPQYSWKRYNILNQEQPLAQPASGQPVSLK

SEQ ID NO: 48 - protein, 55 aa, synthetic construct
  REGION 1..55: note = A33 antigen mass spec fragment 3
DDNTEDKEDARPNREAYEEPPEQLRELSREREEEDDYRQEEQRSTGRESPDHLDQ

SEQ ID NO: 49 - protein, 15 aa, synthetic construct
  REGION 1..15: note = Synthetic peptide
FSNKNYIHGELYKNR

SEQ ID NO: 50 - protein, 19 aa, synthetic construct
  REGION 1..19: note = 31.1 epitope
SPTPQYSWKRYNILNQEQP

SEQ ID NO: 51 - DNA, 965 bp, synthetic construct
  misc_feature 1..965: note = NPC-1 antibody light chain
  source 1..965: mol_type = other DNA
atgagaataccattaattagctagggaccaaaattcaaagacaaaatggattttcaggtgcagattttcagcttcctgctaatcagtgcctcagtcatactgtccagaggacaagttgttctcacccagtctccagtaatcatgtctgcatctccaggggagaaggtcaccatgacctgcagtgccagctcaagtataagttacatgtactggtaccagcagaagccaggcacctcccccaaaagatggatttatgacacatccaaactggcttctggagtccctgctcgcttcagtggcagtgggtctgggacctcttattctctcacaatcagcaacatggaggctggagatgctgccacttattactgccatcagcgggattcttacccatggacgttcggtggaggcaccaacctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagctccccagctccatcctatcttcccttctaaggtcttggaggcttccccacaagcgacctaccactgttgcggtgctccaaacctcctccccacctccttctcctcctcctccctttccttggcttttatcatgctaatatttgcagaaaatattcaataaagtgagtctttgcacttgaaaa

SEQ ID NO: 52 - protein, 235 aa, synthetic construct
  REGION 1..235: note = NPC-1 antibody light chain
  REGION 46..55: note = MISC_FEATURE - CDR1
  REGION 71..77: note = MISC_FEATURE - CDR2
  REGION 110..118: note = MISC_FEATURE - CDR3
MDFQVQIFSFLLISASVILSRGQVVLTQSPVIMSASPGEKVTMTCSASSSISYMYWYQQKPGTSPKRWIYDTSKLASGVPARFSGSGSGTSYSLTISNMEAGDAATYYCHQRDSYPWTFGGGTNLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 10 aa, synthetic construct
  REGION 1..10: note = NPC-1 light chain CDR1
SASSSISYMY

SEQ ID NO: 54 - protein, 7 aa, synthetic construct
  REGION 1..7: note = NPC-1 light chain CDR2
DTSKLAS

SEQ ID NO: 55 - protein, 9 aa, synthetic construct
  REGION 1..9: note = NPC-1 antibody light chain CDR3
HQRDSYPWT

SEQ ID NO: 56 - DNA, 1520 bp, synthetic construct
  misc_feature 1..1520: note = NPC-1 antibody heavy chain
  source 1..1520: mol_type = other DNA
ttttccatcctcttctcatagagcctccatcagaccatggctgtcctggcactgctcctctgcctggtgacattcccaagctgtgtcctgtcccaggtgcagctgaaggagtcaggacctgacctggtggcgccctcacagagcctgtccatcacatgcactgtctcaggattctcattaagcaaatttggtgtaaactgggttcgccagcctccaggaaagggtctggagtggctgggagtaatatggggtgacgggagcacaagttataattcaggtctcatatcaagactgagcatcagcaaggagaactccaagagccaggttttcttaaaactgaacagtctgcaagctgatgacacagccacatactactgtgtcaaaccggggggtgactactggggtcacggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggtcctacaggactctgacacctacctccacccctccctgtataaataaagcacccagcactgccttgggaccctgcaaaaaaaaaaaaaaaa

SEQ ID NO: 57 - protein, 456 aa, synthetic construct
  REGION 1..456: note = NPC-1 antibody heavy chain
  REGION 49..54: note = MISC_FEATURE - CDR1
  REGION 69..84: note = MISC_FEATURE - CDR2
  REGION 114..121: note = MISC_FEATURE - CDR3
MAVLALLLCLVTFPSCVLSQVQLKESGPDLVAPSQSLSITCTVSGFSLSKFGVNWVRQPPGKGLEWLGVIWGDGSTSYNSGLISRLSISKENSKSQVFLKLNSLQADDTATYYCVKPGGDYWGHGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 58 - protein, 6 aa, synthetic construct
  REGION 1..6: note = NPC-1 antibody heavy chain CDR1
SKFGVN

SEQ ID NO: 59 - protein, 16 aa, synthetic construct
  REGION 1..16: note = NPC-1 heavy chain CDR2
VIWGDGSTSYNSGLIS

SEQ ID NO: 60 - protein, 8 aa, synthetic construct
  REGION 1..8: note = NPC-1 antibody heavy chain CDR3
CVKPGGDY

SEQ ID NO: 61 - DNA, 737 bp, synthetic construct
  misc_feature 1..737: note = NPC-1 Chi-antibody light chain
  source 1..737: mol_type = other DNA
gcatagatctgccaccatggactttcaggtccagatatttagctttctattgattagcgcctctgtcattctgagtagggggcaggtggtgctcacccagtctccagtgatcatgtcagcctcaccaggagaaaaagtgactatgacctgctcagcatcctccagcatcagttacatgtactggtaccagcagaagccaggcacctcgcccaagcgttggatctacgatacttccaagctggcaagtggggtacccgcacgcttcagtggaagtggctccggaacctcgtacagtttgaccatttcaaatatggaagctggggacgcagctacatattattgccaccagagagactcctacccgtggaccttcggaggcggtactaatttagagatcaagaggaccgtagccgctccttccgtgttcatctttcccccttccgacgaacaactgaaaagcggtacagcctccgtggtttgtctgctgaacaacttctacccccgggaggctaaagttcagtggaaggttgacaatgctctgcagtcaggcaactctcaagagagcgtcacggagcaagatagcaaagattctacatattctctctcttctacacttacacttagcaaggccgattatgagaagcacaaggtgtatgcctgcgaggtgactcatcagggtctttcttctcctgtcactaaaagcttcaaccgaggcgaatgttgatgaagatcttacg

SEQ ID NO: 62 - protein, 235 aa, synthetic construct
  REGION 1..235: note = NPC-1 chi-antibody light chain with signal peptide
  SIGNAL 1..22
  REGION 46..55: note = MISC_FEATURE - CDR1
  REGION 71..77: note = MISC_FEATURE - CDR2
  REGION 110..118: note = MISC_FEATURE - CDR3
MDFQVQIFSFLLISASVILSRGQVVLTQSPVIMSASPGEKVTMTCSASSSISYMYWYQQKPGTSPKRWIYDTSKLASGVPARFSGSGSGTSYSLTISNMEAGDAATYYCHQRDSYPWTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 10 aa, synthetic construct
  REGION 1..10: note = NPC-1 chi-antibody light chain CDR1
SASSSISYMY

SEQ ID NO: 64 - protein, 7 aa, synthetic construct
  REGION 1..7: note = NPC-1 chi-antibody light chain CDR2
DTSKLAS

SEQ ID NO: 65 - protein, 9 aa, synthetic construct
  REGION 1..9: note = NPC-1 chi-antibody light chain CDR3
HQRDSYPWT

SEQ ID NO: 66 - DNA, 1418 bp, synthetic construct
  misc_feature 1..1418: note = NPC-1 chi-antibody heavy chain
  source 1..1418: mol_type = other DNA
gagcggtaccgccaccatggcagtgctggcccttcttctatgtctggtgaccttcccatcctgcgtcctgagccaggtacaactgaaggagtcgggcccagacctagtggctccgtcacaatcactctccattacgtgcactgtctccggcttctctttgtctaaattcggcgtgaattgggtgcgacagccccccgggaaggggcttgagtggttaggagttatctggggtgacggctcaaccagctacaactcaggactaatctcacgcttgtcaatttcaaaggagaattcaaagtctcaggtgttccttaagctcaactcgctgcaagccgacgataccgcaacctattactgcgtcaaacctggcggggactactggggccatggcacctccgtcacagtgagttccgcatccacaaagggtcccagtgtttttcctttggcgccctctagcaaatcgacatctggcggcacagccgcacttgggtgcttggttaaagactacttccccgaaccggtgacagtatcttggaactctggcgctcttaccagcggagttcataccttccctgccgtattacagtctagcgggccctactccctctcctctgtcgtgacagtcccaagctcttctctgggaactcaaacctacatctgcaatgtgaaccataaacctagcaacacgaaagtggacaaaaaggtcgaacccaagagttgcgacaagacacacacctgccctccttgtcctgctccagagctcctcggcggacctagcgttttcttgttccctccgaaaccaaaggacaccttgatgatttctcggacccccgaggtgacatgtgtagtagttgatgtctcccacgaggaccctgaggtcaagtttaattggtatgtggacggtgtggaggtccacaacgccaaaacaaaaccacgggaggaacagtacaattccacatatagggtggtgagcgtccttaccgtcctgcatcaggattggttaaatggtaaggagtataagtgtaaggtgtctaacaaggctctgcctgctcccatcgaaaaaactataagtaaggccaaaggacagcccagggaacctcaggtgtatactcttccacccagtagagatgagctgactaaaaaccaggtgtccctgacttgtctggtgaagggattttacccatccgatatcgccgtggaatgggagtccaacggacagccagaaaacaattataaaactatgccaccagtgctggatagtgatggtagtttttttctgtacagtaagctgactgttgataagagtagatggcagcagggtaatgtttttagttgtagcgttatgcacgaagctctgcacaatcactatactcagaagagcctgagcctgagccccggtaagtgatgaggtaccgagc

SEQ ID NO: 67 - protein, 462 aa, synthetic construct
  REGION 1..462: note = NPC-1 chi-antibody heavy chain
  SIGNAL 1..19
MAVLALLLCLVTFPSCVLSQVQLKESGPDLVAPSQSLSITCTVSGFSLSKFGVNWVRQPPGKGLEWLGVIWGDGSTSYNSGLISRLSISKENSKSQVFLKLNSLQADDTATYYCVKPGGDYWGHGTSVTVSSASTKGPSVFPLAPSSKSTSGGTAALGCLVKDYFPEPVTVSWNSGALTSGVHTFPAVLQSSGPYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTMPPVLDSDGSFFLYSKLTVDKSRWQQGNVFSCSVMHEALHNHYTQKSLSLSPGK

SEQ ID NO: 68 - protein, 10 aa, synthetic construct
  REGION 1..10: note = NPC-1 chi-antibody heavy chain CDR1
GFSLSKFGVN

SEQ ID NO: 69 - protein, 16 aa, synthetic construct
  REGION 1..16: note = NPC-1 chi-antibody heavy chain CDR2
VIWGDGSTSYNSGLIS

SEQ ID NO: 70 - protein, 5 aa, synthetic construct
  REGION 1..5: note = NPC-1 chi-antibody heavy chain CDR3
PGGDY

SEQ ID NO: 71 - DNA, 735 bp, synthetic construct
  misc_feature 1..735: note = Humanized NEO 103 light chain
  source 1..735: mol_type = other DNA
aagcttgccaccatgaagtacctgctgcccaccgctgctgctggcttgctgctgctggcagctcagcctgccatggccgagatcgtgctgacccagtctcctggcaccctgtctctgagccctggcgagagagctaccctgtcctgctccgcctcctccagcatctcctacatgtactggtatcagcagaagcccggccaggcccctcggctgctgatctacgatacctccaagctggcctccggcatccccgacagattctccggctctggctctggcaccgacttcaccctgaccatctcccggctggaacccgaggacttcgccgtgtactactgccaccagcgggactcctacccctggacctttggccagggcaccaagctggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctctaccctgaccctgtccaaggccgactacgagaagcacaaggtgtacgcctgcgaagtgacccaccagggcctgtctagccccgtgaccaagtctttcaaccggggcgagtgctgatgaggatcctgatga

SEQ ID NO: 72 - protein, 239 aa, synthetic construct
  REGION 1..239: note = Humanized NEO 103 light chain
KLATMKYLLPTAAAGLLLLAAQPAMAEIVLTQSPGTLSLSPGERATLSCSASSSISYMYWYQQKPGQAPRLLIYDTSKLASGIPDRFSGSGSGTDFTLTISRLEPEDFAVYYCHQRDSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 1437 bp, synthetic construct
  misc_feature 1..1437: note = Humanized NEO 103 heavy chain
  source 1..1437: mol_type = other DNA
aagcttgccaccatggacctgctgtgcaagaacatgaagcacctgtggttctttctgctgctggtggccgctcccagatgggtgctgtctcaggtgcagctggtggaatctggccctggcctggtgcagccttccagatccctgtctctgacctgctcctccagcggcttcagcctgtccaagttcggcgtgaactgggtgcgacagcctcctggcaagggcctggaatgggtgggagtgatctggggcgacggctccacctcctacaactccggcctgatctccagagtgaccatctcccgggacacctccaagaaccagctgttcctgaagatggactccctgaccgccgaggacaccgccgtgtactactgtgctagacctggcggcgactactggggccagggcacaacagtgaccgtgtcctccgcttccaccaagggcccctctgtgtttcctctggccccctccagcaagtccacctctggtggaactgccgctctgggctgcctcgtgaaggactacttccccgagcccgtgacagtgtcctggaactctggcgctctgacctccggcgtgcacacctttccagctgtgctgcagtccagcggcctgtactccctgtcctccgtcgtgaccgtgccttccagctctctgggcacccagacctacatctgcaacgtgaaccacaagccctccaataccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcatcaggactggctgaacggcaaagagtacaagtgcaaggtgtccaacaaggccctgcctgcccccatcgaaaagaccatcagcaaggctaagggccagccccgcgagccccaggtgtacacactgcctccatcccgggaagagatgaccaagaatcaggtgtccctgacctgtctcgtgaaaggcttctacccctccgatatcgccgtggaatgggagtccaacggccagcccgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatgatgaggatcctga

SEQ ID NO: 74 - protein, 473 aa, synthetic construct
  REGION 1..473: note = Humanized NEO 103 heavy chain
KLATMDLLCKNMKHLWFFLLLVAAPRWVLSQVQLVESGPGLVQPSRSLSLTCSSSGFSLSKFGVNWVRQPPGKGLEWVGVIWGDGSTSYNSGLISRVTISRDTSKNQLFLKMDSLTAEDTAVYYCARPGGDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 963 bp, synthetic construct
  misc_feature 1..963: note = 16C3 antibody light chain
  source 1..963: mol_type = other DNA
gcggggcagcctcacacagaacacacacagatatgggtgtacccactcagctcctgttgctgtggcttacagtcgtagttgtcagatgtgacatccagatgactcagtctccagcttcactgtctgcatctgtgggagaaactgtcaccatcacatgtggagcaagtgagaatatttacggtgctttaaattggtatcagcggaaacagggaaaatctcctcagctcctgatttatggcgcaagtaatttggcagatggcatgtcatcgaggttcagtggcagtggatctggtagacagtattctctcaagatcagtagcctgcatcctgacgatgttgcaacgtattactgtcaaaatgtattaagtagtccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacaccaacttcacccattgtcaagagcttcaacaggaatgagtgttagagacaaaggtcctgagacgccaccaccagctccccagctccatcctatcttcccttctaaggtcttggaggcttccccacaagcgactaccactgttgcggtgctccaaacctcctccccacctccttctcctcctcctccctttccttggcttttatcatgctaatatttgcagaaaatattcaataaagtgatctttgcacaaaaaaaaaaaaaaaaaaaaaaaaaa

SEQ ID NO: 76 - protein, 107 aa, synthetic construct
  REGION 1..107: note = 16C3 antibody light chain
  REGION 22..32: note = MISC_FEATURE - CDR1
  REGION 48..54: note = MISC_FEATURE - CDR2
  REGION 86..95: note = MISC_FEATURE - CDR3
LMTQSPASLSASVGETVTITCGASENIYGALNWYQRKQGKSPQLLIYGASNLADGMSSRFSGSGSGRQYSLKISSLHPDDVATYYCQNVLSSPYTFGGGTKLEIKKG

SEQ ID NO: 77 - protein, 11 aa, synthetic construct
  REGION 1..11: note = 16C3 light chain CDR1
GASENIYGALN

SEQ ID NO: 78 - protein, 7 aa, synthetic construct
  REGION 1..7: note = 16C3 antibody light chain CDR2
GASNLAD

SEQ ID NO: 79 - protein, 10 aa, synthetic construct
  REGION 1..10: note = 16C3 antibody light chain CDR3
CQNVLSSPYT

SEQ ID NO: 80 - DNA, 1575 bp, synthetic construct
  misc_feature 1..1575: note = 16C3 antibody heavy chain
  source 1..1575: mol_type = other DNA
acgcgggacacagtagtctctacagtcacaggagtacacaggacattgccatgggttggagctgtatcatcttctttctggtagcaacagctacaggtgtgcactcccaggtccagctgcagcagtctgggcctgaggtggtgaggcctggggtctcagtgaagatttcctgcaagggttccggctacacattcactgattatgctatgcactgggtgaagcagagtcatgcaaagagtctcgagtggattggacttattagtacttacagtggtgatacaaagtacaaccagaatttaagggcaaggccacaatgactgtagacaaatcctccaacacagcctatatggaacttgccagattgacatctgaggattctgccatctattactgtgcaagaggggattattccggtagtaggtactggtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgttcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggccctacaggactttgacacctacctccacccctccctgtataaataaagcacccagcactgcctcgggaccctgcataaaaaaaaaaaaaaaaaaaaaaaaaaaa

SEQ ID NO: 81 - protein, 116 aa, synthetic construct
  REGION 1..116: note = 16C3 antibody heavy chain
  REGION 23..32: note = MISC_FEATURE - CDR1
  REGION 47..63: note = MISC_FEATURE - CDR2
  REGION 96..107: note = MISC_FEATURE - CDR3
LEESGPEVVRPGVSVKISCKGSGYTFTDYAMHWVKQSHAKSLEWIGLISTYSGDTKYNQNFKGKATMTVDKSSNTAYMELARLTSEDSAIYYCARGDYSGSRYWFAYWGQGTTVTR

SEQ ID NO: 82 - protein, 10 aa, synthetic construct
  REGION 1..10: note = 16C3 antibody heavy chain CDR1
GYTFTDYAMH

SEQ ID NO: 83 - protein, 17 aa, synthetic construct
  REGION 1..17: note = 16C3 antibody heavy chain CDR2
LISTYSGDTKYNQNFKG

SEQ ID NO: 84 - protein, 12 aa, synthetic construct
  REGION 1..12: note = 16C3 antibody heavy chain CDR3
GDYSGSRYWFAY

SEQ ID NO: 85 - protein, 107 aa, synthetic construct
  REGION 1..107: note = 16C3 antibody light chain (ven16C3)
DIQMTQSPSSLSASVGDRVTITCGASENIYGALNWYQRKPGKSPKLLIYGASNLADGMPSRFSGSGSGRQYTLTISSLQPEDVATYYCQNVLSSPYTFGGGTKLEIK

SEQ ID NO: 86 - protein, 107 aa, synthetic construct; identical to SEQ ID NO: 85
  REGION 1..107: note = 16C3 antibody light chain (cdr16C3)

SEQ ID NO: 87 - protein, 107 aa, synthetic construct
  REGION 1..107: note = 16C3 antibody light chain (abb16C3)
DIQMTQSPSSLSASVGDRVTITCQASENIYGALNWYQRKPGKSPKLLIYGASNLATGMPSRFSGSGSGTDYTFTISSLQPEDIATYYCQNVLSSPYTFGGGTKLEIK

SEQ ID NO: 88 - protein, 107 aa, synthetic construct
  REGION 1..107: note = 16C3 antibody light chain (sdr16C3)
DIQMTQSPSSLSASVGDRVTITCQASENIYGALNWYQRKPGKSPKLLIYGASNLATGMPSRFSGSGSGRQYTFTISSLQPEDIATYYCQQVLSSPYTFGGGTKLEIK

SEQ ID NO: 89 - protein, 107 aa, synthetic construct
  REGION 1..107: note = 16C3 antibody light chain (fra16C3)
DIQMTQSPSSLSASVGDRVTITCCASENIYGALNWYQRKPGKSPNLLIYGASNLADGMPSRFSGSGSGRQYTLTISSLQPEDVATYYCQNVLSSPYTFGGGTKLEIK

SEQ ID NO: 90 - protein, 121 aa, synthetic construct
  REGION 1..121: note = 16C3 antibody heavy chain (ven16C3)
QVQLQQSGPEVVRPGVSVKISCKGSGYTFTDYAMHWVKQSHAKSLEWIGLISTYSGDTKYNQNFKGKATMTVDKSASTAYMELSSLRSEDTAVYYCARGDYSGSRYWFAYWGQGTLVTVSS

SEQ ID NO: 91 - protein, 121 aa, synthetic construct
  REGION 1..121: note = 16C3 antibody heavy chain (cdr16C3)
QVQLQQSGPEVVRPGVSVKISCKGSGYTFTDYAMHWVKQSHAKSLEWIGLISTYSGDTKYNQNFKGKATMTVDTSISTAYMELSRLRSDDTAIYYCARGDYSGSRYWFAYWGQGTLVTVSS

SEQ ID NO: 92 - protein, 121 aa, synthetic construct
  REGION 1..121: note = 16C3 antibody heavy chain (abb16C3)
QVQLQQSGPEVVRPGVSVKISCKGSGYTFTDYAMHWVKQSHAKSLEWIGLISTYSGDTKYSQKFQGRVTMTVDKSASTAYMELSSLRSEDTAVYYCARGDYSGSRYWFAYWGQGTLVTVSS

SEQ ID NO: 93 - protein, 121 aa, synthetic construct
  REGION 1..121: note = 16C3 antibody heavy chain (sdr16C3)
QVQLQQSGPEVVRPGVSVKISCKGSGYTFTDYAMHWVKQSHAKSLEWIGLISTYSGDTKYNQKFQGKATMTVDKSASTAYMELSSLRSEDTAVYYCARGDYSGSRYWFAYWGQGTLVTVSS

SEQ ID NO: 94 - protein, 121 aa, synthetic construct
  REGION 1..121: note = 16C3 antibody heavy chain (fra16C3)
QVQLQQSGPEVVRPGVSVKISCKGSGYTFTDYAMHWVKQSHAKSLEWIGLISTYSGDTKYNQNFKGKATMTVDKSTSTAYMELSSLRSEDTAVYYCARGDYSGSRYWFAYWGQGTLVTVSS

SEQ ID NO: 95 - DNA, 702 bp, synthetic construct
  misc_feature 1..702: note = h16C3-Abb* light chain
  source 1..702: mol_type = other DNA
atgggcgtgcccacccagctgctgctgctgtggctgaccgtggtggtggtgcggtgcgacatccagatgacccagtcccctagctctctgagcgcctccgtgggcgacagggtgaccatcacctgccaagcctctgagaacatctacggcgccctgaactggtaccagaggaagcccggcaagagccccaagctgctgatctacggcgcctctaacctggccaccggcatgcctagccggttctccggctccggcagcggcaccgactacaccttcaccatctcctccctgcaacccgaggacatcgccacctactactgccagcaggtgctgtcctccccctacaccttcggcggcggcaccaaactggagatcaagcggaccgtggccgcccccagcgtgttcatcttccccccctctgacgagcagctgaagtccggcaccgcctctgtggtgtgcctgctgaacaacttctaccccagggaggccaaggtccagtggaaggtggacaacgccctgcagtccggcaacagccaggagtctgtgaccgagcaggactccaaggactccacctacagcctgtctagcaccctgaccctgtccaaggccgactacgagaagcacaaggtgtacgcctgcgaggtgacccaccagggcctgtccagccctgtgaccaagtccttcaacaggggcgagtgctga

SEQ ID NO: 96 - protein, 233 aa, synthetic construct
  REGION 1..233: note = h16C3-Abb* light chain
  REGION 43..53: note = MISC_FEATURE - CDR1
  REGION 69..75: note = MISC_FEATURE - CDR2
  REGION 108..116: note = MISC_FEATURE - CDR3
MGVPTQLLLLWLTVVVVRCDIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 11 aa, synthetic construct
  REGION 1..11: note = h16C3-Abb* light chain CDR1
QASENIYGALN

SEQ ID NO: 98 - protein, 7 aa, synthetic construct
  REGION 1..7: note = h16C3-Abb* light chain CDR2
GASNLAT

SEQ ID NO: 99 - protein, 9 aa, synthetic construct
  REGION 1..9: note = h16C3-Abb* light chain CDR3
QQVLSSPYT

SEQ ID NO: 100 - DNA, 1413 bp, synthetic construct
  misc_feature 1..1413: note = h16C3-Abb* heavy chain
  source 1..1413: mol_type = other DNA
atgggctggtcctgcatcatcttcttcctggtggccaccgccaccggcgtgcacagccaggtgcagcttgtgcagagcggcgccgaggtgaagaagcccggcgccagcgtgaaggtgtcctgcaaggcctccggctacaccttcaccgactacgccatgcactgggtgcggcaggcccccggccagcggctggagtggatgggcctgatcagcacctactctggcgacaccaagtacaaccagaacttccagggccgggtgaccatgaccgtggacaagagcgccagcaccgcctacatggagctgtcctccctgaggtctgaggacaccgccgtgtactactgcgcccggggcgactacagcggcagccggtactggttcgcctactggggccagggcaccctggtgaccgtgtccagcgcctctaccaagggccccagcgtgtttcccctggccccttcctccaaaagcaccagcggcggtaccgccgccctgggctgcctggtgaaggactacttccccgagcccgtgaccgtgagctggaactccggcgccctgaccagcggcgtgcacaccttccctgccgtgctgcaaagctccggcctgtactccctgagctctgtggtgaccgtgccctccagctccctgggcacccagacctacatctgcaacgtgaaccacaagcccagcaacaccaaggtggacaagaaggtggagcctaagtcttgcgacaagacccacacctgccccccttgccctgcccctgagctgctgggcggccccagcgtgttcctgttccctcccaagcccaaggacaccctgatgatctcccggacccctgaggtgacctgcgtggtggtggatgtgagccacgaggatcctgaagtgaagttcaattggtatgtggatggcgtggaggtgcacaacgccaagaccaagccccgggaggagcagtacaacagcacctacagggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcctcccagccgggacgagctgaccaagaaccaggtgtccctgacctgcctggtgaagggcttctacccctctgacatcgccgtggagtgggagagcaacggccagcccgagaacaactacaagaccaccccccccgtgctggactccgacggctccttcttcctgtactctaagctgaccgtggacaagtcccgctggcagcagggcaacgtgttcagctgctccgtgatgcacgaggccctgcacaaccactacacccagaagagcctgagcctgtctcccggcaagtga

SEQ ID NO: 101 - protein, 470 aa, synthetic construct
  REGION 1..470: note = h16C3-Abb* heavy chain
  REGION 45..54: note = MISC_FEATURE - CDR1
  REGION 70..85: note = MISC_FEATURE - CDR2
  REGION 118..129: note = MISC_FEATURE - CDR3
MGWSCIIFFLVATATGVHSQVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 10 aa, synthetic construct
  REGION 1..10: note = h16C3-Abb* heavy chain CDR1
GYTFTDYAMH

SEQ ID NO: 103 - protein, 16 aa, synthetic construct
  REGION 1..16: note = h16C3-Abb* heavy chain CDR2
ISTYSGDTKYNQNFQG

SEQ ID NO: 104 - protein, 12 aa, synthetic construct
  REGION 1..12: note = h16C3-Abb* heavy chain CDR3
GDYSGSRYWFAY

SEQ ID NO: 105 - protein, 236 aa, synthetic construct
  REGION 1..236: note = humanized 31.1 antibody light chain
MKYLLPTAAAGLLLLAAQPAMAEIVMTQSPLSLPVSPGEPASISCKASQSVSNDVAWYLQKPGQSPKLLIYYASNRYTGVPDRFSGSGSGTDFTLKISRVEAEDLGVYYCQQDYSSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 467 aa, synthetic construct
  REGION 1..467: note = humanized 31.1 antibody heavy chain
MDWTWRILFLVAAATGAQAQVQLVQSGPELKKPGASVKVSCKASGYTFTNYGMNWVRQAPGKGLEWMGWINTYTGEPTYADDFKGRFSMSLDTSTSTAYLQISSLKSEDTAVYFCARAYYGKYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - DNA, 738 bp, synthetic construct
  misc_feature 1..738: note = humanized NEO 302 light chain
  source 1..738: mol_type = other DNA
aagcttgccaccatgaagtacctgctgcccaccgctgctgctggcttgctgctgctggcagctcagcctgccatggccgagatcgtgatgacccagtcccctctgtccctgcctgtgtctcctggcgagcctgcctccatctcctgcaaggcctcccagtccgtgtccaacgacgtggcctggtatctgcagaagcctggccagtcccccaagctgctgatctactacgcctccaaccggtacaccggcgtgcccgacagattctccggctctggctctggcaccgacttcaccctgaagatctcccgggtggaagccgaggacctgggcgtgtactactgtcagcaggactactcctcccccctgacctttggccagggcaccaagctggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtccagcaccctgaccctgtccaaggccgactacgagaagcacaaggtgtacgcctgcgaagtgacccaccagggcctgtctagccccgtgaccaagtctttcaaccggggcgagtgctgatgatgaggatcctga

SEQ ID NO: 108 - protein, 240 aa, synthetic construct
  REGION 1..240: note = humanized NEO 302 light chain
KLATMKYLLPTAAAGLLLLAAQPAMAEIVMTQSPLSLPVSPGEPASISCKASQSVSNDVAWYLQKPGQSPKLLIYYASNRYTGVPDRFSGSGSGTDFTLKISRVEAEDLGVYYCQQDYSSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - DNA, 1431 bp, synthetic construct
  misc_feature 1..1431: note = humanized NEO 302 heavy chain
  source 1..1431: mol_type = other DNA
aagcttgccaccatggactggacctggcgcatcctgtttctggtggccgctgctacaggcgcccaggctcaggtgcagctggtgcagtctggacccgagctgaagaaacctggcgcctccgtgaaggtgtcctgcaaggcttccggctacacctttaccaactacggcatgaactgggtgcgacaggcccctggcaagggcctggaatggatgggctggatcaacacctacaccggcgagcccacctacgccgacgacttcaagggccggttctccatgtccctggacacctccaccagcaccgcctacctgcagatctccagcctgaagtccgaggataccgccgtgtacttctgcgccagagcctactacggcaagtacttcgactactggggccagggcaccctcgtgaccgtgtcctctgcttctaccaagggcccctccgtgttccctctggccccttccagcaagtctacctctggcggcacagccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggacccagcgtgttcctgttccccccaaagcccaaggacaccctgatgatctcccggacccccgaagtgacctgcgtggtggtggatgtgtctcacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatgatgaggatcctga

SEQ ID NO: 110 - protein, 471 aa, synthetic construct
  REGION 1..471: note = humanized NEO 302 heavy chain
KLATMDWTWRILFLVAAATGAQAQVQLVQSGPELKKPGASVKVSCKASGYTFTNYGMNWVRQAPGKGLEWMGWINTYTGEPTYADDFKGRFSMSLDTSTSTAYLQISSLKSEDTAVYFCARAYYGKYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 554 bp, synthetic construct
  misc_feature 1..554: note = 4B6 antibody light chain
  source 1..554: mol_type = other DNA
aacctgtggggacattgtgatgtcacagtctccatcctccctggctgtgtcagcaggagaaaaggtcactttgaactgcaaatccagtcagagtctgctcaacagtagaacccgaaagaactacttggcttggtaccagcaaaaaccagggcagtctcctaaattactgatctactgggcatccactagggaatctggggtccctgatcgcttcacaggcagtggatctgggacagatttcactctcaccatcaacagtgtgcaggctgaagacctggcagtttattactgcaagcaatcttataatctcttcacgttcggctcggggacaaagtmgaagtaaaacgggctgatgctgcaccaactgtatccatcttcccaccatccagtgagcagttaacatctggaggtgcctcagtcgtgtgcttcttgaacaacttctaccccaaagacaccaatgtcaagtggaagattgatggcagtgaacgacaaaatggcgtcctgaacagttggactgatcaggacagcaaagacagcacctacagcatgagcag

SEQ ID NO: 112 - protein, 184 aa, synthetic construct
  REGION 1..184: note = 4B6 antibody light chain
  REGION 30..41: note = MISC_FEATURE - 4B6 antibody light chain CDR1
  REGION 59..61: note = MISC_FEATURE - 4B6 antibody light chain CDR2
  REGION 98..105: note = MISC_FEATURE - 4B6 antibody light chain CDR3
TCGDIVMSQSPSSLAVSAGEKVTLNCKSSQSLLNSRTRKNYLAWYQQKPGQSPKLLIYWASTRESGVPDRFTGSGSGTDFTLTINSVQAEDLAVYYCKQSYNLFTFGSGTKLEVKRADAAPTVSIFPPSSEQLTSGGASVVCFLNNFYPKDTNVKWKIDGSERQNGVLNSWTDQDSKDSTYSMS

SEQ ID NO: 113 - protein, 12 aa, synthetic construct
  REGION 1..12: note = 4B6 antibody light chain CDR1
QSLLNSRTRKNY

SEQ ID NO: 114 - skipped

SEQ ID NO: 115 - protein, 8 aa, synthetic construct
  REGION 1..8: note = 4B6 light chain CDR3
KQSYNLFT

SEQ ID NO: 116 - DNA, 351 bp, synthetic construct
  misc_feature 1..351: note = 4B6 heavy chain
  source 1..351: mol_type = other DNA
tgaggtgcagctggaggagtctggagctgaactggcgaggcccggggcttcagtgaagctgtcttgtaaggcttctggctactccttcactgactattatataaattgggtgaagcagaggactggacagggccttgagtggattggagaaatttatcctttaggtggtactagtttctacaatgagaggttcaaggacaaggccacactgactgcagacaaatcctccagcacagtctacatggaactcagcagcctgacatctgaggactcggcagtctatttctgtgcaagaggggataattattacgacgtctactttgactactggggccaagggaccacggtcac

SEQ ID NO: 117 - protein, 111 aa, synthetic construct
  REGION 1..111: note = 4B6 antibody heavy chain
  REGION 26..33: note = MISC_FEATURE - CDR1
  REGION 51..58: note = MISC_FEATURE - CDR2
  REGION 97..109: note = MISC_FEATURE - CDR3
EVQLEESGAELARPGASVKLSCKASGYSFTDYYINWVKQRTGQGLEWIGEIYPLGGTSFYNERFKDKATLTADKSSSTVYMELSSLTSEDSAVYFCARGDNYYDVYFDYWG

SEQ ID NO: 118 - protein, 8 aa, synthetic construct
  REGION 1..8: note = 4B6 heavy chain CDR1
GYSFTDYY

SEQ ID NO: 119 - protein, 8 aa, synthetic construct
  REGION 1..8: note = 4B6 heavy chain CDR2
IYPLGGTS

SEQ ID NO: 120 - protein, 13 aa, synthetic construct
  REGION 1..13: note = 4B6 heavy chain CDR3
ARGDNYYDVYFDY

SEQ ID NO: 121 - DNA, 21 bp, synthetic construct
  misc_feature 1..21: note = siRNA ID# s9074 (sense)
  source 1..21: mol_type = other DNA
agatgtgcctcaactacgatt

SEQ ID NO: 122 - DNA, 21 bp, synthetic construct
  misc_feature 1..21: note = siRNA ID# s9074 (antisense)
  source 1..21: mol_type = other DNA
tcgtagttgaggcacatcttg

SEQ ID NO: 123 - DNA, 21 bp, synthetic construct
  misc_feature 1..21: note = siRNA ID# s9075 (sense)
  source 1..21: mol_type = other DNA
gctctggaacgtgagcatatt

SEQ ID NO: 124 - RNA, 21 nt, synthetic construct
  misc_feature 1..21: note = siRNA ID# s9075 (antisense)
  source 1..21: mol_type = other RNA
tatgctcacgttccagagccg

SEQ ID NO: 125 - DNA, 21 bp, synthetic construct
  misc_feature 1..21: note = siRNA ID# s9076
  source 1..21: mol_type = other DNA
gcgtgctcgtcgacaactatt

SEQ ID NO: 126 - RNA, 21 nt, synthetic construct
  misc_feature 1..21: note = siRNA ID# s9076 (antisense)
  source 1..21: mol_type = other RNA
tagttgtcgacgagcacgcgg

SEQ ID NO: 127 - DNA, 21 bp, synthetic construct
  misc_feature 1..21: note = siRNA ID# S2885 (sense)
  source 1..21: mol_type = other DNA
agaactcagtgagtgcaaatt

SEQ ID NO: 128 - RNA, 21 nt, synthetic construct
  misc_feature 1..21: note = siRNA ID#S2885 (antisense)
  source 1..21: mol_type = other RNA
tttgcactcactgagttctgg

SEQ ID NO: 129 - DNA, 21 bp, synthetic construct
  misc_feature 1..21: note = siRNA ID#S9285
  source 1..21: mol_type = other DNA
ggaacgatgcaggatcctatt

SEQ ID NO: 130 - DNA, 21 bp, synthetic construct
  misc_feature 1..21: note = siRNA ID#S9285 (antisense)
  source 1..21: mol_type = other DNA
taggatcctgcatcgttcctt

(19) United States
(12) Patent Application Publication
(10) Pub. No.: US 2025/0326859 A1
(43) Pub. Date: Oct. 23, 2025

(54) ANTI-CANCER ANTIBODIES, COMBINATION THERAPIES, AND USES THEREOF

(71) Applicant: PRECISION BIOLOGICS, INC., Bethesda, MD (US)

(72) Inventor: Phil M. ARLEN, Bethesda, MD (US)

(21) Appl. No.: 19/039,402

(22) Filed: Jan. 28, 2025

Related U.S. Application Data
(62) Division of application No. 17/667,867, filed on Feb. 9, 2022, now abandoned.

(51) Int. Cl. C07K 16/30; A61K 39/395; A61K 45/06; A61N 5/10

(52) U.S. Cl. CPC C07K 16/30; A61K 39/39558; A61K 45/06; C07K 16/3015; C07K 16/3023; C07K 16/303; C07K 16/3046; C07K 16/3069; A61N 2005/1021; A61N 2005/1087; A61N 2005/1089; A61N 2005/109; A61N 2005/1098; C07K 2317/24; C07K 2317/56; C07K 2317/565; C07K 2317/732

(57) ABSTRACT

This invention relates to antibodies to anti-cancer antibodies and uses thereof, including combination therapies and compositions. Combinations targeting the extrinsic, intrinsic, or common pro-apoptotic pathways are provided. Exemplary anti-cancer antibodies are differentially expressed in cancers, such as antibodies targeting the bind the antigens NPC-1, 16C3, or 31.1. Exemplary anti-cancer antibodies target lung cancer, ovarian cancer, cervical cancer, uterine cancer, pancreas cancer, breast cancer, and colon cancer.

### RELATED APPLICATION DISCLOSURE

This application claims the benefit of U.S. Provisional Patent Application Ser. No. 62/142,770, filed Apr. 3, 2016, which is hereby incorporated by reference in its entirety.

### SEQUENCE DISCLOSURE

This application includes as part of its disclosure a biological sequence listing contained in the file “43282o4202.txt” and having a size of 212,792 bytes, created on Apr. 4, 2016, which is hereby incorporated by reference in its entirety.

### FIELD

The present application generally relates to the field of cancer diagnostics and therapeutics. Exemplary embodiments provide methods wherein cancer is detected, diagnosed, monitored, and/or treated. Exemplary treatment methods involve combination therapies, which may include an antibody as disclosed herein and another anti-cancer agent. Said other anti-cancer agent may target the extrinsic apoptosis pathway, intrinsic apoptosis pathway, or the common apoptosis pathway.

### BACKGROUND

Cancer is caused by a malfunction in the growth control systems of a cell. Cells control their growth via combination of proliferation inhibition by tumor suppressor genes (e.g., Retinoblastoma protein (pRb), p53) and proliferation activation by oncogenes (proto-oncogenes) (e.g., RAS, WNT, MYC, EKR, and TRK). A mutation in either a tumor suppressor gene and/or a protooncogene in a cell results in unusually high rates of cell proliferation (e.g., a tumor cell). See Knudson (1971) Proc. Natl. Acad. Sci. USA 68(4): 820-823. The cell may exhibit early signs of aberrant growth such as aberrant morphology or unusually large size (hyperplasia). The tumor cells also may proliferate at a higher than usual but not lethal rate, forming a growth, known as benign tumor (dysplasia). In later stages of cancer, the tumor cells proliferate at an unusually high rate resulting in uncontrolled growth that threatens the health of the patient known as malignant tumors (or in situ cancer). Many tumors can “metastasize” or spread throughout the body forming tumors. Metastasis is generally a sign of late stage, terminal cancer. Weinberg (September 1996) “How Cancer Arises” Scientific American 62-70.

Many cancer therapies exert their antitumor effect by triggering apoptosis in cancer cells. Stress-inducible molecules, for example, c-Jun N-terminal kinase (JNK), mitogen-activated protein kinase (MAPK)/extracellular signal-regulated protein kinase (ERK), nuclear factor kappa B (NF-κB) or ceramide, have been implied in transmitting apoptotic signals. Proteolytic enzymes including caspases are important effector molecules in apoptosis. The caspases are a family of cysteine proteases that act as death effector molecules in various forms of cell death.

Co-owned prior applications have described multiple cancer-associated antigens and specific antibodies thereto, e.g., in the following U.S. and PCT applications and U.S. patents: WO/2012/040617, WO/2011/163401, WO/2009/062050, WO/2006/113546, U.S. Pat. Nos. 7,829,678, 7,763,720, and 7,314,622, and U.S. pre-grant publication nos. 2012/0034227, 2011/0165599, 2011/0158902, 2011/0129416, 2011/0076761, 2010/0310559, 2009/0162931, and 2008/0227965, each of which is hereby incorporated by reference in its entirety. These include cancer-specific antigens MUC5AC, CEACAM5, CEACAM6, and the A33 antigen, more specifically the NPC-1 antigen on the MUC5AC protein, the 16C3 antigen on CEACAM5 and CEACAM6 proteins, and 31.1 epitope on the A33 protein. Exemplary antibodies are described in Table 1 below with the full antibody sequences being furnished herewith.

### MUC5AC

MUC5AC, a mucin, is an example of a cancer-specific antigen. Mucins are high molecular weight glycoproteins with O-linked oligosaccharides attached to serine or threonine residues of the apomucin protein backbone expressed in a cell and tissue-specific pattern in normal tissues. The mucin family includes proteins that contain tandem repeat structures with a high proportion of prolines, threonines, and serines (which constitute the PTS domain). Mucins are further defined by extensive glycosylation of the PTS domain through GalNAc O-linkages at the threonine and serine residues. Each mucin has a central region with a variable number of tandem repeat with about eight amino acid residues, but there is a little similarity. There are two structurally and functionally distinct classes of mucins: secreted gel-forming mucins and transmembrane mucins. Secreted gel-forming mucins include the products of the MUC2, MUC5AC, MUC5B and MUC6 genes. See Kocer, et al. (2006) BMC Gastroenterology 6: 4; See also Hollingsworth & Swanson (2004) Nature Reviews 4: 45-60.

The human mucin (MUC) family consists of members designated MUC1 to MUC21—subclassified into secreted and transmembrane forms. The secreted mucins (e.g., MUC2, MUC5AC, MUC5B and MUC6) form a physical barrier, which acts as a mucous gel that provides protection for epithelial cells that line the respiratory and gastrointestinal tracts and form the ductal surfaces of organs such as the liver, breast, pancreas, and kidney. The transmembrane mucins (e.g., MUC1, MUC4, MUC 13 and MUC 16) have a single membrane-spanning region and contribute to the protective mucous gel through their ectodomains of (9-glycosylated tandem repeats that form rod-like structures. Kufe (2009) Nature Reviews 9: 874-885. MUC5AC expression is found on apical epithelial cells of the mucus glands of gastric antrum and body, tracheobronchial epithelium, superficial epithelium of the gallbladder and endocervix epithelium.

MUC5AC is highly expressed in adenoma. See Kocer, et al. (2006) BMC Gastroenterology 6: 4. Additionally, MUC5AC is expressed in tumors of gastrointestinal, pancreaticobiliary, and endocervical origin (e.g., colon, esophagus, liver, lung, pancreas, stomach, and uterus). See Lau, et al. (2004) Am. J. Clin Pathol. 122: 61-69. MUC5AC is also highly expressed in breast and gastric cancers. Zhang, et al. (1998) Clinical Cancer Research 4: 2669-2676. Further, MUC5AC glycan variants have been associated with pancreatic NEOplasms. Haab, et al. (May 2010) Annals of Surgery 251(5): 937-945. MUC5AC is aberrantly expressed by colorectal polyps and colorectal carcinoma. Kocer, et al. (2006) BMC Gastroenterology 6(4): 1-9.

### CEACAM5 and CEACAM6

CEACAM 5 and CEACAM6 are additional examples of cancer-specific antigens. The carcinoembryonic antigen (CEA) gene family is a member of the IgCAM superfamily including 29 related genes and pseudogenes. CEA proteins function as intercellular hemophilic and heterophilic adhesion molecules and have signaling properties. Carcinoembryonic cell adhesion molecule (CEACAM) 5 and CEACAM6 share −90% homology in the N domain but differ in the number of IgC2-like domains (A and B domains). Both proteins contain a glycosylphosphatidylinositol (GPI) membrane anchor and are targeted to lipid rafts in apical membranes of polarized epithelial cells. CEACAM5 and CEACAM6 bind a variety of gram-negative bacteria and mediate internalization/phagocytosis, participating in innate immune defense in the intestine. Kolla, et al. (2009) Am J Physiol Lung Cell Mol Physiol 296: L1019-L1030; Lund, et al. (2003) Cancer Gene Therapy 10: 365-376.

CEACAM5 and CEACAM6 are overexpressed in many cancers (e.g., breast, ovarian, colon, pancreatic, lung, and prostate). CEACAM5 and CEACAM6 are believed to be involved in cell adhesion, cellular invasiveness, resistance to anoikis, and metastatic behavior of tumor cells. Zhang, et al. (1998) Clinical Cancer Research 4: 2669-2676; Strickland, et al. (2009) Journal of Pathology 218: 380-390; Blumenthal, et al. (2005) Cancer Research 65(19): 8809-8817; Blumenthal, et al. (2007) BMC Cancer 7(2): 1-15.

### A33 Antigen Protein

A33 is another example of a cancer-specific antigen. The A33 antigen is a cell surface glycoprotein expressed in the small intestine and colonic epithelium. The A33 antigen shares homology with tight-junction associated proteins of the immunoglobulin superfamily including CAR and JAM. A33 antigen is expressed in 95% of colon tumors but not normal intestine or other organs. Ackerman, et al. (2008) Cancer Immunol Immunother 57(7): 1017-1027; Garinchesa, et al. (1996) Int. J. Oncol. 9(3): 465-71.

### SUMMARY

In one aspect, the present invention relates to therapeutic use of antibodies to cancer-associated antigens (“anti-CAA antibodies”), such as NEO-201 antibodies, NEO-102 antibodies, or NEO-301 antibodies. Said anti-CAA antibodies may be used in combination with another therapeutic agent or regimen, preferably resulting in enhanced therapeutic efficacy.

In another aspect the present invention relates to the selection of patients for treatment in a therapeutic regimen involving the use of antibodies to cancer-associated antigens, such as NEO-201 antibodies. Said patient may be a patient with a cancer (such as such as breast, ovarian, cervical, or uterine cancer). The patient may be selected for treatment based upon the presence of a cancer at a specified stage, such as pre-cancer and Stage I, II, II and IV cancers including metastatic cancers. Said cancer may express the antigen bound by said anti-CAA antibody (such as NEO-201), e.g., cancer or pre-cancer of the colon, pancreas, lung (e.g., mesothelioma), prostate, skin (e.g., melanoma), breast, ovary, cervix, or uterus, or a metastatic cancer cells originating from said tissue or organ.

In another aspect the present invention relates to combination therapies for treatment of cancers that express a cancer-associate antibody. Preferred combination therapies include treatment with a subject anti-CAA antibody, such as a NEO-201 antibody, in combination with another therapeutic agent or regimen. Such treatments result in enhanced therapeutic efficacy relative to the individual therapeutic agents. Without intent to be limited by theory, it is believed that the subject anti-cancer antibodies are able to trigger apoptotic pathways, such that a combination with an agent or regimen that also target apoptosis can result in enhanced cancer cell killing. Such combination therapies may result in therapeutic effects such as promoting tumor regression, enhanced cell killing, or increasing patient survival.

Thus, in one aspect, the present disclosure provides therapeutic methods comprising treatment with an anti-CAA antibody (such as NEO-201) and another agent that targets (i.e., activates) the intrinsic apoptosis pathway.

In another aspect, the present disclosure provides therapeutic methods comprising treatment with an anti-CAA antibody (such as NEO-201) and another agent that targets (i.e., activates) the extrinsic apoptosis pathway.

One group of such agents that target the extrinsic pathway agents bind to death receptors, such as death receptor ligands (as well as fragments or analogs thereof). Additional exemplary agents that bind to death receptors include anti-death receptor antibodies, e.g., agonistic antibodies that bind to and activate said death receptor. Agonistic antibodies may also sensitize said death receptor to activation by another ligand, e.g., an endogenous or exogenous death receptor ligand. Exemplary agents include agents that target PML-RARα, DR4 (TRAIL R1), and/or DR5 (TRAIL R2). Examples of agents targeting the extrinsic pathway include TRAIL (human TRAIL polypeptide or an agonistic fragment thereof), Dr4 agonists, Dr5 agonists, and all trans retinoic acid (ATRA). Dr4 and Dr5 agonists include agonistic anti-Dr4 and anti-Dr5 monoclonal antibodies, respectively, such as Apomab, HGS-ETR1, HGS-ETR2, and HGS-TR2J.

In some embodiments, an agent that activates the extrinsic apoptotic pathway refers to substances that induce apoptosis by binding to death receptors, e.g., ligands. Exemplary ligands of death receptors are tumor necrosis factor a (TNF-alpha), tumor necrosis factor (TNF-beta, lymphotoxin alpha), lymphotoxin beta (LT-beta), TRAIL (Apo2L), CD95 (Fas, APO-I) ligand, TRAMP (DR3, Apo-3) ligand, DR4 ligand, DR6 ligand as well as fragments, variants, and derivatives of said ligands.

As noted, one ligand that activates the extrinsic apoptotic pathway is TRAIL (Apo2L). “TRAIL” (TNF-related apoptosis-inducing ligand) refers to a cytokine that is produced and secreted by most normal tissues cells. The full-length human TRAIL polypeptide is a 281 amino acid long, Type II transmembrane protein. See UniProtKB/Swiss-Prot Accession No. P50591. TRAIL causes apoptosis by binding to the death receptors DR4 and DR5. The terms “Apo2L/TRAIL,” “Apo2L,” “Apo-2 ligand” and “TRAIL” are used herein to refer to the TRAIL polypeptide sequence as well as biologically active fragments, deletional, insertional, or substitutional variants thereof. In some embodiments, the fragments or variants are biologically active and have at least about 80% amino acid sequence identity, more preferably at least about 90% sequence identity, and even more preferably, at least 95%, 96%, 97%, 98%, or 99% sequence identity with the human TRAIL sequence. This definition encompasses substitutional variants of TRAIL in which at least one of its native amino acids are substituted by an alanine residue. These substitutional variants include those identified, for example, as “D203A,” “D218A” and “D269A.” This nomenclature is used to identify Apo2L/TRAIL variants wherein the aspartic acid residues at positions 203, 218, and/or 269 are substituted by alanine residues. See U.S. Pat. No. 7,741,282. Optionally, the TRAIL variants may include one or more of the alanine substitutions, which are recited in Table I of WO 01/00832. Substitutional variants include one or more of the residue substitutions identified in Table I of WO 01/00832. The definition also encompasses a native sequence of TRAIL isolated from a TRAIL source or prepared by recombinant or synthetic methods. The TRAIL of the invention includes the polypeptides referred to as Apo2L/TRAIL or TRAIL disclosed in WO 97/01633 and WO 97/25428. In some embodiments, the TRAIL of the invention is Superkiller-TRAIL, as described by Wang, et al. ((2004) Cancer Cell 5:501). The terms “Apo2L/TRAIL” or “Apo2L” are also used to refer generally to forms of TRAIL that include monomer, dimer or trimer forms of the polypeptide. The person skilled in the art knows that the aforementioned proteins may be produced using standard techniques for the production of recombinant proteins. Alternatively, recombinant Apo2L/TRAIL is commercially available, for example from Prospec (East Brunswick, N.J.).

In other embodiments, an agent that activates the extrinsic apoptotic pathway refers to an antibody directed against one or more cellular death receptors. In particular embodiments, the antibody is a monoclonal antibody that binds to cellular death receptors and has been shown to induce cell death in different types of tumor cells. Examples of such antibodies include, but are not limited to, anti-CD95 antibody, anti-TRAIL-R1 (DR4) antibody, anti-TRAIL-R2 (DR5) antibody, anti-DR6 antibody, anti TNF-R1/2 antibody and anti-TRAMP (DR3) antibody as well as fragments or derivatives thereof. In some embodiments, the antibody is an anti-TRAIL-R1 (D4) antibody. An exemplary anti-TRAIL-R1 (D4) antibody includes, but is not limited to, mapatumumab (HGS-ETR1). Mapatumumab is an agonistic monoclonal antibody to TRAIL-R1 with apoptosis promoting and potential antitumor activities. Mapatumumab selectively binds to and activates the TRAIL cell receptor, thereby inducing apoptosis and reducing tumor growth. In another embodiment, the antibody is an anti-TRAIL-R2 (D5) antibody. An exemplary anti-TRAIL-R2 (D5) antibody includes, but is not limited to, lexatumumab (HGS-ETR2). Lexatumumab is a fully human monoclonal agonistic antibody directed against TRAIL-R2 with potential antitumor activity. Mimicking the natural ligand TRAIL, lexatumumab binds to and activates TRAIL-R2, which may trigger apoptosis in and inhibit the growth of TRAIL-R2-expressing tumor cells. Additional monoclonal antibodies that bind cellular death receptors include conatumumab (AMG655), dulanermin (AMG 951, APO2L/TRAIL, PRO1762, RG3639, rhApo2L/TRAIL), tigatuzumab (CS1008), TRAIL R (DR4-Specific Altrimer, Anaphore), HGS TR2J, LBY135, drozitumab (PR085780, apomab), SL231, SM164 with TRAIL R2, TAS266, and the like.

In still other embodiments, an agent that activates the extrinsic apoptotic pathway refers to a chemotherapeutic agent that has been shown to activate the extrinsic apoptotic pathway. For example, treatment with DNA-damaging agents such as doxorubicin, etoposide, cisplatin or bleomycin have been shown to trigger an increase in CD95L expression, which stimulates the receptor pathway in an autocrine or paracrine manner by binding to its receptor CD95 (Friesen, et al. (1996) Nat. Med. 2:574-577; Fulda, et al. (1997) Cancer Res. 57:3823-3829; Fulda, et al. (1998) Int. J. Cancer 76:105-114; Houghton, et al. (1997) Proc. Natl. Acad. Sci. USA 94:8144-8149; Muller, et al. (1997) J. Clin. Invest. 99:403-413). The CD95 receptor/ligand system has also been implicated in thymine-less death in colon carcinoma cells following treatment with 5-fluorouracil (Houghton, et al. (1997) supra). Furthermore, upregulation of FADD and procaspase-8 has been found upon treatment with doxorubicin, cisplatin or mitomycin C in colon carcinoma cells (Micheau, et al. (1999) Biophys. Res. Commun. 256:603-611). Moreover, oxaliplatin has been shown to increase caspase-8 activity and increase Bid expression in colorectal cancer cells (DiCesare, et al. (2013) Free Radic. Biol. Med. 61C:143-150). Accordingly, in certain embodiments, the agent that activates the extrinsic apoptotic pathway is oxaliplatin.

Yet another extrinsic pathway agent is 2-deoxy-D-glucose, which has been reported to sensitize cancer cells to agents that activate the extrinsic apoptotic pathway (see U.S. Pub. No. 20140377274, which is hereby incorporated by reference in its entirety).

Further extrinsic pathway agents include drugs that target a Fas pathway, a c-FLIP pathway, 4-(4-Chloro-2-methylphenoxy)-N-hydroxybutanamide (CMH), estradiol (E2), or delta-tocotrienol.

Agents that target the intrinsic pathway include agents that target Bcl-1, Bcl-XL, Bax, BCL-Xs and/or PML-RARα. These include agents that act directly on the mitochondrial inner membrane, agents that antagonize the antiapoptotic members of the Bcl-2 protein family, and agents that enhance the activity of the proapoptotic members of the Bcl-2 family of proteins such as Bax. Examples of agents targeting the intrinsic pathway include arsenic trioxide, lonidamine (a derivative of indazole-3-carboxylic acid), antisense agents targeting Bcl-1 (such as Genasense, G3139 or oblimersen sodium), antisense agents targeting Bcl-XL, Bax, and BCL-Xs. Additional antisense agents target both Bcl-2 and Bcl-XL, or target clusterin (also known as testosterone-repressed prostate message 2). Exemplary intrinsic pathway agents also include small molecules. One group of small molecules recognizes the surface pocket of Bcl-2 or Bcl-XL, including Antimycin-A and derivatives thereof, HA14-1, and synthetic BH3 organic peptides. Additional intrinsic pathway agents include farnesyl-thiosalicylic acid (FTS), estradiol (E2), delta-tocotrienol, salinomycin, and curcumin

It is to be understood that “antisense agents” refers to short interfering RNAs (siRNAs) and a number of functionally similar compound classes that utilize RNA interference (RNAi) to downregulate expression of a target gene.

Additional therapeutic agents that may be used in combination with one or more anti-CAA antibody as disclosed herein (including NEO-201, NEO-102, and NEO-301 antibodies) antimetabolites, alkylators, corticosteroids, radiation, monoclonal antibodies, platins and PARP inhibitors. Exemplary combinations include one or more of said anti-CAA antibodies (such as NEO-201) together with epirubicin, cisplatin, dacarbazine, fludarabine/cyclophosphamide, dexamethasone, doxorubicin, or other anti-cancer agents known in the art. It is to be understood that said combination may be provided together in a single formulation, or may be suitable for co-administration. Thus, methods of co-administering said agents may be provided wherein the anti-CAA antibody (such as NEO-201) and another therapeutic agent may be administered at the same time or at different times, wherein therapeutically effective dosages of both the anti-CAA antibody and said other agent may be delivered to the patient.

Further exemplary agents that may be utilized in combination with the subject anti-CAA antibodies (such as NEO-201) include FTS, CMH, TMS, and estradiol (E2). FTS invokes caspase-dependent death in cancer cells through the mitochondrial cell death pathway. FTS promotes apoptosis in MCF-7 cells and tumor xenografts. CMH is a small molecule inhibitor of Cellular FLICE (FADD-like IL-1beta-converting enzyme)-inhibitory protein (c-FLIP) and CMH can activate caspase-8 and -10 by inhibiting c-FLIP. Part of the mechanism of CMH's ability to sensitize cells to death ligands is through its ability to inhibit HDAC3, HDAC6 and HDAC8. TMS is an agent that invokes a predominantly caspase-independent death through the mitochondrial death pathway via microtubule inhibition. TMS is effective for reducing the growth of TamR resistant breast cancer tumor xenografts. Estradiol was shown to induce apoptosis of long term estrogen deprived cells through the mitochondrial cell death pathway and also the Fas death receptor pathway. Estradiol promotes apoptosis of long-term estrogen deprived cells in vitro, in xenograft models as well as patients.

Further exemplary anti-cancer agents include cytostatic agents, cytocidal agents, actinomycin D, adriamycin, arsenic trioxide, asparaginase, bleomycin, busulfan, camptosar, carboplatinum, carmustine, chlorambucil, cisplatin, corticosteroids, colicheamicin, cyclophosphamide, daunorubicin, docetaxel, doxorubicin, epirubicin, etoposide, fludarabine, fluorouracil, gemcitabina, gemcitabine, gemzar, hydroxyurea, idarubicin, ifosfamide, irinotecan, lomustine, melphalan, mercaptomurine, methotrexate, mitomycin, mitoxantrone, oxaliplatin, paclitaxel, platinol, platinex, procarbizine, raltitrexeel, rixin, steroids, streptozocin, taxol, taxotere, thioguanine, thiotepa, tomudex, topotecan, treosulfan, trihydrate, vinblastine, vincristine, vindesine, vinorelbina, vinorelbine, duanomycin, dactinomysin, esorubisin, mafosfamide, cytosine arabinoside, bis-chloroethylnitrosurea, mitomycin C, mithramycin, prednisone, hydroxyprogesterone, testosterone, tamoxifen, dacarbazine, hexamethylmelamine, pentamethylmelamine, amsacrine, chlorambudil, methylcyclohexylnitrosurea, nitrogen mustards, cyclophosphamide, 6-mercaptopurine, 6-thioguanine, cytarabine, 5-azacytidine, deoxyco-formycin, 4-hydroxyperoxycyclophosphoramide, 5-fluorouracil (5-FU), 5-fluorodeoxyuridine (5-FUdR), colchicine, trimetrexate, teni-poside, and diethylstilbestrol.

Still further exemplary anti-cancer agents include DNA damaging agents, nucleophosmin, agents which induce cellular damage as part of a synergistic process with another agent, a catalytic antibody, prodrugs, CHK1/2 inhibitor, CBP-501, AZD7762, histone deacetylase inhibitor, vorinostat, tumour necrosis factor related apoptosis inducing ligand, BH3 mimetic, ABT737, small molecule inhibitors, tyrosine kinase inhibitors, imatinib mesylate, gefitinib, erlotinib, monoclonal antibodies, rituximab and trastuzumab.

For example, other therapies or anticancer agents that may be used in combination with the inventive compounds of the present invention include, but are not limited to, surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, antibodies, aptamers, siRNAs, oligonucleotides, enzyme, ion channel and receptor inhibitors or activators to name a-few), hyperthermia and cryotherapy, agents to attenuate any adverse effects (e.g., antiemetics), and other approved chemotherapeutic drugs, including, but not limited to, alkylating drugs (e.g., mechlorethamine, chlorambucil, Cyclophosphamide, Melphalan, Ifosfamide), antimetabolites (e.g., Methotrexate), purine antagonists and pyrimidine antagonists (e.g., 6-Mercaptopurine, 5-Fluorouracil, Cytarabile, Gemcitabine), spindle poisons (e.g., Vinblastine, Vincristine, Vinorelbine, Paclitaxel), podophyllotoxins (e.g., Etoposide, Irinotecan, Topotecan), antibiotics (Doxorubicin, Bleomycin, Mitomycin), nitrosoureas (e.g., Carmustine, Lomustine), inorganic ions (e.g., Cisplatin, Carboplatin), enzymes (e.g., Asparaginase), and hormones (e.g., Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/dmglistfrarne).

Another composition and therapeutic regimen involve the combination of an anti-CAA antibody (such as NEO-201) with a common pathway agent. Common pathway agents are a group of agents that target both the extrinsic and intrinsic pathways, or target elements downstream of both the extrinsic and intrinsic pathway. Common pathway agents may target caspase, or other shared or common members of the extrinsic and extrinsic pathways. Exemplary common pathway agents include caspase activators, apoptin, and survivin.

Yet another composition and therapeutic regimen involve the combination of an anti-CAA antibody (such as NEO-201) with an apoptosis pathway agent. Apoptosis pathway agents (also referred to as agents that target an apoptotic pathway) are a group of agents are believed to promote apoptosis, which may potentially sensitize cells to other killing agents. These include without limitation common, extrinsic, and extrinsic pathway agents, such as agents that target p53, p53 pathway members, IκB kinase (e.g., inhibitors or antagonists thereof), IKKβ, the proteasome/ubiquitin pathway (including the 20S proteasome), the PI3K/Akt pathway (such as mTOR). Exemplary apoptosis pathway agents also include, without limitation thereto, ONY-015, INGN201, PS1145, Bortezomib, CCI779, RAD-001, and antisense therapy targeting MDM2 (which is a regulator of p53 activity).

A further composition and therapeutic regimen involve the combination of an anti-CAA antibody (such as NEO-201) with a direct cell killing agent. Direct cell killing agents include the protein mixed lineage kinase domain like (MLKL), rapamycin (RAP) or derivatives and/or analogs thereof, such as everolimus or RAD001; CCI-779, ABT578, SAR543, ascomycin (an ethyl analog of FK506), AP23573, AP23841, KU-0063794, INK-128, EX2044, EX3855, EX7518, or compounds that bind to the ATP-binding cleft of mTOR, such as AZD08055 and OSIO27.

Additionally, as further disclosed herein, combination therapies comprising an anti-CAA antibody (such as NEO-201) and exemplary other therapeutic agents are predicted to have enhanced therapeutic efficacy compared to the individual therapeutic agents. Based thereon, it is further predicted that lower dosages of said other therapeutic agent(s) can achieve the therapeutic efficacy in combination with said anti-CAA antibody (such as NEO-201), thereby allowing therapeutic benefit at a lower dosage to decrease side-effects.

The invention further provides a kit comprising said anti-CAA antibody (such as NEO-201) and said other agent. Typically said anti-CAA antibody (such as NEO-201) and/or said other agent are provided at therapeutically effective dosages for the treatment of a disease or condition, e.g., cancer such as breast, ovarian, uterine, or cervical cancer.

The invention further provides pharmaceutical compositions comprising said anti-CAA antibody (e.g., NEO-201, NEO-301, or NEO-102 antibodies) and one or more agents that target an apoptotic pathway (such as the common, extrinsic or intrinsic apoptotic pathway). In certain embodiments, the pharmaceutical compositions of the present invention further comprise one or more additional therapeutically active ingredients (e.g., chemotherapeutic and/or palliative), for example one or more agents that target an apoptotic pathway (such as the common, extrinsic or intrinsic apoptotic pathway). For purposes of the invention, the term “palliative” refers to treatment that is focused on the relief of symptoms of a disease and/or side effects of a therapeutic regimen, but is not curative. For example, palliative treatment encompasses painkillers, antinausea medications, anti-pyretics, and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

The present compounds and compositions can be administered together with hormonal and steroidal anti-inflammatory agents, such as but not limited to, estradiol, conjugated estrogens (e.g., PREMARIN, PREMPRO, AND PREMPHASE), 17 beta estradiol, calcitonin-salmon, levothyroxine, dexamethasone, medroxyprogesterone, prednisone, cortisone, flunisolide, and hydrocortisone; non-steroidal anti-inflammatory agents, such as but not limited to, tramadol, fentanyl, metamizole, ketoprofen, naproxen, nabumetone, ketorolac, tromethamine, loxoprofen, ibuprofen, aspirin, and acetaminophen; anti-TNF-alpha antibodies, such as infliximab (REMICADE) and etanercept (ENBREL).

The pharmaceutical compositions of the present invention comprise a therapeutically effective amount of a compound of the present invention formulated together with one or more pharmaceutically acceptable carriers and one or more agents that target an apoptotic pathway (such as the common, extrinsic or intrinsic apoptotic pathway). As used herein, the term “pharmaceutically acceptable carrier” means a non-toxic, inert solid, semi-solid or liquid filler, diluent, encapsulating material or formulation auxiliary of any type. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

TABLE 1
Antibodies which selectively bind NPC-1, 16C3, or 31-1 epitopes.

| Antibody | Aliases | Antigen | Exemplary SEQ ID NOs | Description |
|---|---|---|---|---|
| NPC-1 | | NPC-1 | | Murine hybridoma that expresses |
| | | | | NPC-1 IgG1 (ATCC) |
| NEO-101 | NPC-1C, | NPC-1 | Light Chain (SEQ ID NOs: 51, 52) | Chimeric NPC-1 antibody, |
| | ensituximab | | LC CDRs (SEQ ID NOs: 53-55) | engineered in CHO-DG44 |
| | | | Heavy Chain (SEQ ID NOs: 56, 57) | production cell clone 4B7; |
| | | | HC CDRs (SEQ ID NOs: 58-60) | targets a variant of MUCSAC |
| NEO-102 | | NPC-1 | Light Chain (SEQ ID NOs: 61, 62) | Chimeric NPC-1 antibody, |
| | | | LC CDRs (SEQ ID NOs: 63-65) | engineered in CHO-M production |
| | | | Heavy Chain (SEQ ID NOs: 66, 67) | cells, contains 2 amino acid |
| | | | HC CDRs (SEQ ID NOs: 68-70) | changes in HC constant domain* |
| NEO-103 | | NPC-1 | Light Chain (SEQ ID NOs: 71, 72) | Humanized NPC-1 antibody |
| | | | Heavy Chain (SEQ ID NOs: 73, 74) | |
| 16C3 | | 16C3 | Light Chain (SEQ ID NOs: 75, 76) | Murine hybridoma that expresses |
| | | | LC CDRs (SEQ ID NOs: 77-79) | 16C3 IgG1 (ATCC) |
| | | | Heavy Chain (SEQ ID NOs: 80, 81) | |
| | | | HC CDRs (SEQ ID NOs: 82-84) | |
| 16C3 | Variant h16C3 | 16C3 | Light Chain (SEQ ID NOs: 85-89) | Humanized 16C3 antibody |
| | antibodies | | Heavy Chain (SEQ ID NOs: 90-94) | |
| NEO-201 | h16C3-Abb* | 16C3 | Light Chain (SEQ ID NOs: 95, 96) | Humanized 16C3 antibody |
| | | | LC CDRs (SEQ ID NOs: 97-99) | |
| | | | Heavy Chain (SEQ ID NOs: 100, 101) | |
| | | | HC CDRs (SEQ ID NOs: 102-104) | |
| 31.1 | | 31.1 | | Chimeric 31.1 antibody, produced |
| | | | | in CHO-K cells |
| NEO-301 | 31.1C | 31.1 | Light Chain (SEQ ID NOs: 105) | Chimeric 31.1 antibody, contains |
| | | | Heavy Chain (SEQ ID NOs: 106) | 2 amino acid changes in HC |
| | | | | constant domain*, produced |
| | | | | in high titer CHO-S cells |
| NEO-302 | | 31.1 | Light Chain (SEQ ID NOs: 107, 108) | Humanized 31.1 antibody |
| | | | Heavy Chain (SEQ ID NOs: 109, 110) | |

*2 amino acid changes in the heavy chain constant domain are Proline at residue 175 to Leucine in CH1 and Methionine at residue 390 to Threonine in CH3.

In addition the present invention methods for using specific antibodies and fragments thereof to detect cancer specific antigens in vitro and in vivo are provided herein. The use of these antibodies to stage cancer prognosis, design specific treatment regimens, and to establish the efficacy of a specific treatments is also provided.

Further provided is the use of these antibodies or fragments thereof, in naked or conjugated form, alone or in association with other cancer treatment compositions (such as and one or more agents that target an apoptotic pathway (such as the common, extrinsic or intrinsic apoptotic pathway), for treating different human cancers corresponding to the specific human cancer cell lines disclosed herein.

These antibodies, NEO-101 (previously referred to by the Assignee as NPC-1), NEO-102, NEO-201 (previously referred to by the Assignee as h16C3-Abb*) and NEO-301 (previously referred to as 31.1) are summarized in Table 1 and the sequences thereof are further described herein.

NEO-101 specifically binds to an epitope comprised in MUC5AC, in particular it recognizes a repeated epitope comprised in the MUC5AC antigen that is specifically expressed on different human tumors which epitope comprises specific carbohydrate residues (“glycotope”). NEO-201 specifically binds to epitopes comprised of specific amino acid residues which are comprised in the CEACAM5 and CEACAM6 antigens, which antigens and the corresponding NEO-201 epitopes are also expressed on different human tumors. Finally, NEO-301 specifically binds to an epitope contained in the A33 antigen which antigen and corresponding epitope is similarly expressed by different human tumors.

NEO-102 is a genetically engineered chimeric monoclonal antibody that has been manufactured from a recombinant Chinese hamster ovary (CHO) cell production clone. In an exemplary embodiment this antibody may be formulated at 10.0 mg/mL in 25 mM sodium citrate, 150 mM sodium chloride, 0.1% polysorbate-80.

NEO-201 is a genetically engineered humanized monoclonal antibody that has been manufactured from a recombinant CHO cell production clone. In an exemplary embodiment this antibody may be formulated at 10.4 mg/mL in 20 mM sodium phosphate, 150 mM sodium chloride pH7.2.

NEO-301 a genetically engineered chimeric monoclonal antibody that has been manufactured from a recombinant CHO cell production clone. In an exemplary embodiment this antibody may be formulated at 11.5 mg/mL in 20 mM sodium phosphate, 150 mM sodium chloride pH7.2.

### BRIEF DESCRIPTION OF THE DRAWINGS

FIG. 1. NEO-101, NEO-201 and NEO-301 Antigen Expression by Human Normal and Cancer Cell Lines. NEO-101, NEO-201 and NEO-301 specific antigen expression in various cell lines were detected by Flow Cytometry. Viable cell suspensions were incubated with primary antibody NEO-101, NEO-201 and NEO-301 separately at 10 ug/mL. Human IgG (10 ug/mL) was used as negative control. Goat anti-human IgG (Fc)-phycoerythrin conjugate was used for primary antibody detection; 7-ADD was used for excluding dead cells from analysis. The staining was measured by Flow Cytometry (Guava instrument, Millipore). Median fluorescent intensity (MFI) and % of stained cells was used for determining presence of specific antigen on each cell line.

FIG. 2. Immunohistochemistry results from cancer tissue arrays. Immunohistochemistry method (IHC) was used for determining the presence of NEO-101/102, NEO-201 and NEO-301 antigens on paraffin microarrays and tissues. These primary antibodies (NEO-101/NEO-102, NEO-201 and NEO-301) were biotinylated prior to use. Paraffin tissue Microarrays and slides of tissues were reacted with the primary Ab after inactivating endogenous peroxidase and blocking the slides. Staining was detected with streptavidin-horseradish peroxidase conjugate and visualized with chromogen. Biotinylated human IgG1 was used as negative control and mouse anti-human cytokeratin monoclonal antibody was used as positive control. Antibody staining is measured as % and intensity of tumor epithelial cells and luminal secretion within the whole tissue section.

FIG. 3. Summary of NEO-101, NEO-102 and NEO-301 IHC Results from normal colon and pancreatic paraffin tissues. Normal cell samples as indicated were stained using similar methods as in FIG. 2 above. ADJ: adjacent.

### DETAILED DESCRIPTION

The present disclosure provides anti-cancer antibodies which selectively bind NPC-1, 16C3, or 31-1 epitopes, which may comprise NEO-101, NEO-102, NEO-103, NEO-201, NEO-301, NPC-1, 16C3, or 31-1 having polypeptide sequences identified in Table 1, supra, or a variant thereof.

The present disclosure also provides therapeutic compositions comprising said anti-cancer antibody and another therapeutic agent, such as one or more agents that target an apoptotic pathway (such as the common, extrinsic or intrinsic apoptotic pathway). Preferred agents, when co-administered with the anti-cancer antibody, result in enhanced therapeutic efficacy in the patient, e.g., increased cancer cell killing, tumor regression, and/or increased patient survival.

The present disclosure also provides therapeutic methods comprising administering said anti-cancer antibody and another therapeutic regimen to a patient in need thereof. Said therapeutic regimen may include administration of an anti-cancer agent. Said therapeutic regimen may include radiotherapy.

Preferably said anti-cancer agent targets one or more apoptotic pathways in the cell, e.g., the intrinsic pathway, extrinsic pathway, or the common pathway. Said agent may target a molecule that participates in more than one of the extrinsic, intrinsic, and common pathways. Without intent to be limited by theory, it is believed that NEO-201 and other anti-CAA antibodies (such as NEO-201) disclosed herein are able to kill cells by activating one or more apoptotic pathways, such that a combination with an agent or regimen that targets the apoptotic pathway can result in enhanced cancer cell killing. Such combination therapies may result in therapeutic effects such as promoting tumor regression, enhanced cell killing, or increasing patient survival.

Said cancer may express the antigen bound by said anti-CAA antibody (such as NEO-201), e.g., cancer or pre-cancer of the colon, pancreas, lung (e.g., mesothelioma), prostate, skin (e.g., melanoma), breast, ovary, uterus, cervix, or a metastatic cancer cells originating from said tissue or organ.

Exemplary embodiments of the invention provide a therapeutic method comprising administering an effective amount of said modified antibody to a patient, optionally in combination with another therapy such as and one or more agents that target an apoptotic pathway (such as the common, extrinsic or intrinsic apoptotic pathway). Said modified antibody may also contain one or more modifications which increase effector functions. For example, said antibody may contain one or more modifications that increase ADCC, which may result in increased ADCC relative to the same antibody sequence lacking said modification. As a further example, said antibody may contain one or modifications that result in increased binding to one or more Fc receptors, thereby resulting in increased Fc receptor binding relative to the same antibody sequence lacking said modification. As a yet further example, said antibody may contain one or more modifications that result in increased CDC, thereby resulting in increased CDC relative to the same antibody sequence lacking said modification.

In another aspect, the disclosure provides a therapeutic method comprising administering an effective amount of an antibody to a cancer-associated antigen in combination with one or more chemotherapeutic agents such as oxaliplatin, irinotecan, topotecan, leucovorin, carmustine, vincristine, fluorouracil, streptozocin, and gemcitabine, wherein said antibody is preferably an antibody to the NPC-1, 16C3, or 31.1 antigen, such as one of the antibodies identified in Table 1 or a fragment or variant thereof and optionally one or more agents that target an apoptotic pathway (such as the common, extrinsic or intrinsic apoptotic pathway). In yet another aspect, the disclosure provides a therapeutic method comprising administering an effective amount of an antibody to a cancer-associated antigen and optionally one or more agents that target an apoptotic pathway (such as the common, extrinsic or intrinsic apoptotic pathway) in combination with radiation therapy wherein said antibody is preferably an antibody to the NPC-1, 16C3, or 31.1 antigen, such as one of the antibodies identified in Table 1 or a fragment or variant thereof. Said antibody may or may not contain an Fc modification that increases ADCC or another effector function (e.g., as described above). Preferably said antibody in combination exhibits an enhanced or synergistic effect on treatment outcome relative to said antibody alone.

The term “apoptosis” refers to programmed cell death mediated by biochemical pathways that can be induced by various means. A “pro-apoptotic” agent or drug is a bioactive agent or drug that produces a biochemical effect that results in programmed cell death. Apoptosis can be caused or induced by intrinsic or extrinsic pathways or mechanisms, as further described herein. The “extrinsic” apoptosis pathway involves death receptors, and this pathway is activated by ligands that bind to the death receptors. The “intrinsic” apoptosis pathway involves mitochondrial pathways that initiate apoptosis.

Apoptosis is believed to be initiated through the extrinsic and intrinsic pathways (reviewed in Fulda et al., Oncogene (2006) 25, 4798-4811; Ghobrial et al., CA: A Cancer Journal for Clinicians, Volume 55, Issue 3, pages 178-194, May/June 2005, each of which is hereby incorporated by reference in its entirety). The extrinsic pathway (also referred to as the cytoplasmic pathway) can be triggered through the Fas death receptor and includes the membrane-bound Fas ligand (FasL), Fas complexes, and the Fas-associated death domain. Activation of the extrinsic pathway can be initiated with the ligation of cell surface receptors called death receptors (DRs). Death receptors of the tumor necrosis factor (TNF) receptor superfamily include CD95 (APO-1/Fas) or TNF-related apoptosis-inducing ligand (TRAIL), TNF receptor 1 (TNFRI), and TRAIL-R2. When a death stimulus triggers the pathway, the membrane-bound FasL interacts with the inactive Fas complexes and forms a death-inducing signaling complex. The Fas death-inducing signaling complex contains the adaptor protein Fas-associated death domain protein and caspases 8 and 10 and leads to activation of caspase 8, which in turn can activate the rest of the downstream caspases. In some cells, the activation of caspase 8 may be sufficient to execute death, while in other cell types, caspase 8 may interact with the intrinsic apoptotic pathway by cleaving Bid (a proapoptotic member of the Bcl-2 family), leading to the subsequent release of cytochrome-c.

The intrinsic pathway (also called the mitochondrial pathway) involves the release of cytochrome-c from the mitochondria and activation of the death signal, involving Bcl-2 family proteins, Apaf-1, and activation of procaspase-9 to caspase-9.

The Bcl-2 family includes proapoptotic members such as Bax, Bak, Bad, Bcl-Xs, Bid, Bik, Bim, and Hrk, and antiapoptotic members such Bcl-2, Bcl-XL, Bcl-W, Bfl-1, and Mcl-1. Antiapoptotic Bcl-2 members are believed to act as repressors of apoptosis by blocking the release of cytochrome-c, whereas proapoptotic members act as promoters. These effects are though to be more dependent on the balance between Bcl-2 and Bax than on Bcl-2 quantity alone. More generally, the intrinsic pathway may be activated by stimulating or agonizing a pro-apoptotic member of the intrinsic pathway, and/or by antagonizing or downregulating anti-apoptotic members of the intrinsic pathway.

Both the extrinsic and intrinsic pathways converge to a final common or overlapping pathway involving the activation of caspases that cleave regulatory and structural molecules and culminate in the death of the cell.

Experimentally, cell killing can be considered to occur through the extrinsic pathway if it is dependent on caspase 8 but less dependent on or independent from caspase 3. Conversely, cell killing is considered to occur through the intrinsic pathway if it is dependent on caspase 3 but less dependent on or independent from caspase 8. Activation of either caspase may be detected using a luciferase-based caspase assay such as Caspase-Glo®, e.g., to measure activity of caspases-3/7 (Caspase-Glo® 3/7 Assay Systems, Promega) or caspase-8 (Caspase-Glo® 8 Assay System, Promega). Detected activation of a given caspase provides evidence of its involvement in cell killing by an anti-CAA antibody (such as NEO-201) or other agent. Additionally, Caspase 3 and caspase 8 expression may experimentally manipulated (such as using RNAi knock-down) to experimentally test whether a cell killing mechanism depends on one or the other of caspase 3 and caspase 8, and thus identify a cell killing mechanism as dependent on the extrinsic or intrinsic pathway. On the other hand, cell killing that does not depend on caspase 3 or caspase 8 is considered to involve direct cell killing.

RNA interference (RNAi) broadly refers to silencing or “knock down” the expression of a targeted gene. The mechanism of gene silencing in RNAi is believed to be based on degrading or otherwise preventing the transcription or translation of mRNA in a sequence specific manner. In terms of the application of this technology to selectively knocking down gene expression, exogenous double stranded RNA (dsRNA) (including structural analogs of RNA) specific to a gene sought to be knocked down can be introduced into the intracellular environment. Mechanisms by which RNAi molecules can interfere with gene expression have been described in the literature. In brief, it has been reported that once the dsRNA enters the cell, it is typically cleaved by an RNaseIII-like enzyme, Dicer, into double stranded small interfering RNAs (siRNAs) 21-23 nucleotides in length that contain 2 nucleotide overhangs on the 3′ ends. In an ATP dependent step, the siRNAs become integrated into a multi-subunit protein complex known as the RNAi induced silencing complex (RISC), which guides the siRNAs to the target RNA sequence. The siRNA unwinds and the antisense strand remains bound to RISC and directs degradation of the complementary target mRNA sequence by a combination of endo- and exonucleases. However, whereas the RNAi mechanism was originally identified in the context of its role as a microbial defence mechanism in higher eukaryotes, it is also known that RNAi based gene expression knockdown can also function as a mechanism to regulate endogenous gene expression for research or therapeutic purposes. MicroRNA (miRNA) is a form of endogenous single-stranded RNA which is typically 20-25 nucleotides and is endogenously transcribed from DNA, but not translated into protein. The DNA sequence that codes for an miRNA gene generally includes the miRNA sequence and an approximate reverse complement. When this DNA sequence is transcribed into a single-stranded RNA molecule, the miRNA sequence and its reverse-complement base pair to form a double stranded RNA hairpin loop, this forming the primary miRNA structure (pri-miRNA). A nuclear enzyme cleaves the base of the hairpin to form pre-miRNA. The pre-miRNA molecule is then actively transported out of the nucleus into the cytoplasm where the Dicer enzyme cuts 20-25 nucleotides from the base of the hairpin to release the mature miRNA.

The term “short interfering nucleic acid,” “siNA,” “short interfering RNA,” “siRNA,” “short interfering nucleic acid molecule,” “short interfering oligonucleotide molecule,” or “chemically modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Bass, 2001, Nature 411, 428-429; Elbashir et al., 2001, Nature 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science 297, 1818-1819; Volpe et al., 2002, Science 297, 1833-1837; Jenuwein, 2002, Science 297, 2215-2218; and Hall et al., 2002, Science 297, 2232-2237; Hutvagner and Zamore, 2002, Science 297, 2056-60; McManus et al., 2002, RNA 8, 842-850; Reinhart et al., 2002, Gene & Dev. 16, 1616-1626; and Reinhart & Bartel, 2002, Science 297, 1831). For example, the siNA may be a double-stranded polynucleotide molecule comprising complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof.

Exemplary short interfering nucleic acids include long double stranded RNA (dsRNA), hairpin double stranded RNA (hairpin dsRNA), short interfering RNA (siRNA), short hairpin RNA (shRNA), micro RNA/small temporal RNA (miRNA/stRNA), miRNAs which mediate spatial development (sdRNAs), the stress response (srRNAs) or cell cycle (ccRNAs), RNA oligonucleotides designed to hybridize and prevent the functioning of endogenously expressed miRNA or stRNA or exogenously introduced siRNA. RNAi molecules for silencing a given gene of interest can be designed by methods known in the art. In addition to exogenous introduction of an RNAi molecule, a cell may also be caused to express a given RNAi molecule from an exogenously introduced expression construct, wherein desired the sequences are operably linked to an inducible or constitutive promoter. Said expression construct may introduced into said cell in a manner resulting in stable (e.g., genomic integration) or transient (e.g., extrachromosomal) presence of the construct. Additionally included are modified RNA analog molecules containing a sugar backbone or a non-sugar backbone. A sugar backbone may comprise any naturally occurring sugar as well as non-naturally occurring sugars. Examples of naturally occurring sugars include, but are not limited to, ribose, deoxyribose, and/or 2-deoxyribose. Sugar units of a backbone may be modified such that the modified sugar backbone is resistant to cleavage. The sugars of a backbone may be modified by methods known in the art, for example, to achieve resistance to nuclease cleavage. Examples of modified sugars include, but are not limited to, 2′-O-alkyl riboses, such as 2′-O-methyl ribose, and 2′-O-allyl ribose. The sugar units may be joined by phosphate linkers. Typical sugar units of the invention may be linked to each other by 3′-5′, 3′-3′, or 5′-5′ linkages. Additionally, 2′-5′ linkages are also possible if the 2′ OH is not otherwise modified. A non-sugar backbone may comprise any non-sugar molecule to which bases may be attached. Non-sugar backbones are known in the art. Examples include, but are not limited to, morpholino and peptide nucleic acids (PNAs). A morpholino backbone is made up of morpholino rings (tetrahydro-1,4-oxazine) and may be joined by non-ionic phosphorodiamidate groups. Modified morpholinos known in the art may be used in the present invention. PNAs result when bases are attached to an amino acid backbone by molecular linkages. Examples of such linkages include, but are not limited to, methylene carbonyl, ethylene carbonyl, and ethyl linkages. The amino acids may be any amino acid, natural or non-natural, modified or unmodified, and are preferably alpha amino acids. The amino acids may be identical or different from one another. One non-limiting example of a suitable amino acid includes an amino alkyl-amino acid, such as (2-aminoethyl)-amino acid.

Extrinsic Pathway. “Agents that target the extrinsic pathway,” “agents that target the extrinsic apoptotic pathway” “agents that activate the extrinsic pathway,” “agents that activate the extrinsic apoptotic pathway,” “extrinsic pathway agents,” “extrinsic apoptotic pathway agents” (as well as variants of these terms) are used interchangeably herein and refer to the group of agents that activate or sensitize the extrinsic apoptotic pathway of a cell, such as a cancer cell.

Intrinsic Pathway. “Agents that target the intrinsic pathway,” “agents that target the intrinsic apoptotic pathway” “agents that activate the intrinsic pathway,” “agents that activate the intrinsic apoptotic pathway,” “intrinsic pathway agents,” “intrinsic apoptotic pathway agents” (as well as variants of these terms) are used interchangeably herein and refer to the group of agents that activate or sensitize the intrinsic apoptotic pathway of a cell, such as a cancer cell.

### Dosage Forms

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying agents, suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In order to prolong the effect of a drug, it may be desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, disorders are treated or prevented in a subject, such as a human or other animal, by administering to the subject a therapeutically effective amount of a compound of the invention, in such amounts and for such time as is necessary to achieve the desired result. The term “therapeutically effective amount” of a compound of the invention, as used herein, means a sufficient amount of the compound so as to decrease the symptoms of a disorder in a subject. As is well understood in the medical arts a therapeutically effective amount of a compound of this invention will be at a reasonable benefit/risk ratio applicable to any medical treatment.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight (0.05 to 4.5 mg/m²). An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

In certain embodiments, a therapeutic amount or dose of the compounds of the present invention may range from about 0.1 mg/kg to about 500 mg/kg (about 0.18 mg/m² to about 900 mg/m²), alternatively from about 1 to about 50 mg/kg (about 1.8 to about 90 mg/m²). In general, treatment regimens according to the present invention comprise administration to a patient in need of such treatment from about 10 mg to about 1000 mg of the compound(s) of this invention per day in single or multiple doses. Therapeutic amounts or doses will also vary depending on route of administration, as well as the possibility of co-usage with other agents.

In certain embodiments, a composition may comprise active agents, such as an antibody of the disclosure and one or more agents that target an apoptotic pathway (such as the common, extrinsic or intrinsic apoptotic pathway), in amounts that exhibit an enhanced or synergistic effect relative to said antibody alone. Said enhanced or synergistic effect may be determined based on observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration to a subject suffering from or susceptible to a disease or disorder.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the invention and a co-agent, are both administered to a patient simultaneously in the form of a single entity or dosage. The term “non-fixed combination” means that the active ingredients, e.g., a compound of the invention and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The agents or salts thereof may be formulated into pharmaceutical compositions for administration to animals or humans. These pharmaceutical compositions, which comprise an amount of the protein inhibitor effective to treat or prevent a cancer and a pharmaceutically acceptable carrier, are another embodiment of the present invention.

Radiation therapy (also referred to as radiotherapy) is the medical use of radiation to treat malignant cells, such as cancer cells. This radiation can have an electromagnetic form, such as a high-energy photon, or a particulate form, such as an electron, proton, neutron, or alpha particle. A common form of radiation used in practice today is high-energy photons. Photon absorption in human tissue is determined by the energy of the radiation, as well as the atomic structure of the tissue in question. The basic unit of energy used in radiation oncology is the electron volt (eV); 10\hat{ }3 eV=1 keV, 10\hat{ }6 eV=1 MeV. Three interactions can be involved in photon absorption in tissue: the photoelectric effect, Compton effect, and pair production.

In the photoelectric effect, an incoming photon transfers energy to a tightly bound electron. The photon transfers practically all of its energy to the electron and ceases to exist. The electron departs with most of the energy from the photon and begins to ionize surrounding molecules. This interaction depends on the energy of the incoming photon, as well as the atomic number of the tissue; the lower the energy and the higher the atomic number, the more likely that a photoelectric effect will take place. The energy range in which the photoelectric effect predominates in tissue is about 10-25 keV.

The Compton effect is the most important photon-tissue interaction for the treatment of cancer. In this case, a photon collides with a “free electron,” i.e, one that is not tightly bound to the atom. Unlike the photoelectric effect, in the Compton interaction both the photon and electron are scattered. The photon can then continue to undergo additional interactions, albeit with a lower energy. The electron begins to ionize with the energy given to it by the photon. The probability of a Compton interaction is inversely proportional to the energy of the incoming photon and is independent of the atomic number of the material. The Compton effect dominates in the range of about 25 keV-25 MeV and is therefore the most common interaction occurring clinically, as most radiation treatments are performed at energies of about 6-20 MeV.

In pair production, a photon interacts with the nucleus of an atom. The photon gives up energy to the nucleus and, in the process, creates a positron-electron pair of particles. The positive electron (positron) ionizes until it combines with a free electron in positron-electron annihilation. This positron-electron annihilation generates two photons that travel in opposite directions. The probability of pair production is proportional to the logarithm of the energy of the incoming photon and is dependent on the atomic number of the material. The energy range in which pair production dominates is greater than or equal to 25 MeV. This interaction occurs to some extent in routine radiation treatment with high-energy photon beams.

With the advent of high-energy linear accelerators, electrons have become a viable option in treating superficial tumors up to a depth of about 5 cm. Electron depth dose characteristics are unique in that they produce a high skin dose but exhibit a falloff after only a few centimeters.

Electron absorption in human tissue is greatly influenced by the presence of air cavities and bone. The most common clinical uses of electron beams include the treatment of skin lesions, such as basal cell carcinomas, and boosting of areas that have previously received photon irradiation, such as postoperative lumpectomy or mastectomy scar in breast cancer patients, as well as select nodal areas in the head and neck.

Dose computation algorithms are used for radiation therapy planning to help ensure that the desired dose is delivered to a specific patient. Dose computation includes two parts: a source model and a transport model. The source model provides the incident fluence. The transport model computes the dose that results from the incident fluence. The three main transport algorithms in the order of increasing accuracy/decreasing performance are pencil beam, superposition/convolution, and Monte Carlo. Superposition/convolution is the current clinical standard method of calculating radiation dose for external beam radiation therapy.

In recent years, treatment quality has been increased by the use of intensity modulation. This technique uses a multi-leaf collimator to define multiple apertures from a single beam direction providing the ability to vary the intensity of radiation across the beam. This technique allows conforming radiation treatment to the shape of the target and avoid critical structures while drastically increasing the number of beam parameters. The treatment planning system can optimize, through multiple iterations of dose calculations, an objective function having the drastically increased number of beam parameters. In practice, the treatment planner repeats the optimizations multiple times in order to achieve the best results possible for the patient.

This clinical workflow extends to more complex techniques such as volumetric modulated arc therapy (Otto, K., Med. Phys. 35, 310-317, 2008), intensity modulated arc therapy (Yu, C. X., Phys. Med. Biol. 40, 1435-1449, 1995), and adaptive radiation therapy (Yan, D., Vicini, F., Wong, J., Martinez, A, Phys. Med. Biol. 42, 123-132, 1997). Real-time radiation therapy (the ability to scan, re-plan and treat every patient daily) may also be performed. A thorough review of dose calculation in radiation therapy is available from Ahnesjo et al. (Ahnesjo, A., Aspradakis, M, Phys. Med. Biol. 44, R99-R155 1999).

Additionally provided are diagnostic methods comprising administering said modified antibody to a patient and detecting said antibody in said patient.

Exemplary embodiments of the invention provide compositions comprising said modified antibody (such as therapeutic compositions or diagnostic compositions), which compositions may comprise a pharmaceutically acceptable carrier, and additionally may comprise one or more additional therapeutic agents, such as an anti-cancer agent.

Further exemplary embodiments of the invention provide nucleic acids encoding said modified antibodies are provided. Additionally provided are cells (such as mammalian, prokaryotic, yeast, or other eukaryotic cells) or vectors comprising a nucleic acid encoding said modified antibody.

Additional exemplary embodiments of the invention provide methods of making said modified antibodies, comprising expressing a nucleic acid encoding said modified antibody.

In another aspect, this disclosure provides methods of detecting a cancer cell that expresses a cancer-associated antigen. Detecting the expression of cancer-associated antigen (e.g., using an antibody to MUC5AC, CEACAM5, CEACAM6, or the A33 antigen) may be used for diagnosis and staging of cancers (e.g., in radioimaging). For example, the level or extent of expression of one or more of these cancer-associated antigens may indicate the stage of cancer, may be correlated with patient outcome, or may be predictive of the outcome of different treatment options.

Currently, depending on the stage of the cancer, cancer treatment involves one or a combination of the following therapies: surgery to remove the cancerous tissue, radiation therapy, and chemotherapy. Detection of cancer cells using an antibody to a cancer-associated antigen (such as an antibody to MUC5AC, CEACAM5, CEACAM6, or the A33 antigen) can be used in conjunction with one or more therapies. Therapy may be targeted to the cancer cells thereby promoting effective treatment and/or reducing the effect on normal non-cancerous tissue. For example, cells expressing a cancer-associated antigen may be targeted using radiotherapy, surgery, and/or cryotherapy. The therapeutic course (e.g., regimen and dosages of radiotherapy, surgical plan, or course of cryotherapy) that are therapeutically effective will depend on the particular cancer being treated, the extent of the disease and other factors familiar to the physician of skill in the art and can be determined by the physician.

Expression of a cancer-associated antigen (such as MUC5AC, CEACAM5, CEACAM6, or the A33 antigen) may be evaluated using an in vivo diagnostic assay, e.g., by administering a molecule (such as an antibody) which binds the molecule to be detected and is tagged with a detectable label (e.g., a radioactive isotope or a fluorescent label) and externally scanning the patient for localization of the label. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody. Aside from the above assays, various in vivo and in vitro assays for detecting the presence of a cancer-associated antigen are available to the skilled practitioner.

The cancer-associated antigen (such as MUC5AC, CEACAM5, CEACAM6, or the A33 antigen) may be present on the cell surface. Alternatively or in addition the cancer-associated antigen (or a precursor thereof) may be produced and secreted at detectable levels. For example, the cancer-associated antigen may be detected in a biological fluid such as serum, e.g, using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)).

The antibodies may also be used for purification or immunoprecipitation of the cancer-associated antigen from cells or other samples, for detection and quantitation of cancer-associated antigen in vitro, e.g., in an ELISA or a Western blot, to kill and eliminate cancer-associated antigen-expressing cells from a population of mixed cells, e.g., as a step in the purification of other cells.

In another aspect, the invention provides a diagnostic kit comprising a NPC-1, 16C3, or 31.1 antibody such as a NEO-102, NEO-201, or NEO-301 antibody or variant thereof. In one embodiment, the polypeptide may be directly or indirectly fixed to a solid phase support, such as a bead, plate, matrix, polymer, test tube, sheet, culture dish, or test strip. In another embodiment, the solid support may be an array.

In another embodiment, the invention provides an antibody which binds, preferably specifically, to any of the above or below described polypeptides. Optionally, the antibody is a monoclonal antibody, antibody fragment, chimeric antibody, humanized antibody, single-chain antibody or antibody that competitively inhibits the binding of an antibody disclosed herein to its respective antigenic epitope. Antibodies of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like.

The antibodies may optionally be produced in mammalian cells (such as CHO cells), bacterial cells, yeast cells, or other cells or using cell-free methods as known in the art.

For diagnostic purposes, the antibodies of the present invention may be detectably labeled, attached to a solid support, or the like. For example, the antibody may be labeled by conjugation to a radiolabel such as ¹¹¹In or ⁸⁶Y. In exemplary embodiments the ¹¹¹In or ⁸⁶Y (or another radiolabel) may be conjugated to an antibody using the acyclic chelate CHX-A″-DTPA. These radiolabels and conjugation methods are only intended to be illustrative, as alternative radiolabels and/or alternative conjugation methods may be utilized.

In yet another embodiment, the invention concerns an article of manufacture comprising a container and a composition of matter contained within the container, wherein the composition of matter may comprise an antibody as described herein (such as an antibody to MUC5AC, CEACAM5, CEACAM6, or the A33 antigen). The article may further optionally comprise a label affixed to the container, or a package insert included with the container, that refers to the use of the composition of matter for the therapeutic treatment or diagnostic detection of a tumor.

Another embodiment of the present invention is directed to a method for inhibiting the growth of a cell that expresses a cancer-associated antigen as described herein (such as MUC5AC, CEACAM5, CEACAM6, or the A33 antigen), wherein the method comprises contacting the cell with an antibody that binds to the cancer-associated antigen and with one or more agents that target an apoptotic pathway (such as the common, extrinsic or intrinsic apoptotic pathway), and wherein the binding of the antibody to the cancer-associated antigen causes inhibition of the growth of the cell expressing the cancer-associated antigen. In preferred embodiments, the cell is a cancer cell and binding of the antibody to the cancer-associated antigen causes death of the cell expressing the cancer-associated antigen. Optionally, the antibody is a monoclonal antibody, antibody fragment, chimeric antibody, humanized antibody, or single-chain antibody. Antibodies employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies employed in the methods of the present invention may optionally be produced in CHO cells or bacterial cells.

Yet another embodiment of the present invention is directed to a method of therapeutically treating a mammal having a cancerous tumor comprising cells that express a cancer-associated antigen as described herein (such as MUC5AC, CEACAM5, CEACAM6, or the A33 antigen), wherein the method comprises administering to the mammal a therapeutically effective amount of an antibody that binds to the cancer-associated antigen and one or more agents that target an apoptotic pathway (such as the common, extrinsic or intrinsic apoptotic pathway), thereby resulting in the effective therapeutic treatment of the tumor. Optionally, the antibody is a monoclonal antibody, antibody fragment, chimeric antibody, humanized antibody, or single-chain antibody. Antibodies employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies employed in the methods of the present invention may optionally be produced in CHO cells or bacterial cells.

Yet another embodiment of the present invention is directed to a method of determining the presence of a cancer-associated antigen as described herein (such as MUC5AC, CEACAM5, CEACAM6, or the A33 antigen) in a sample suspected of containing the cancer-associated antigen, wherein the method comprises exposing the sample to an antibody that binds to the cancer-associated antigen and determining binding of the antibody to the cancer-associated antigen in the sample, wherein the presence of such binding is indicative of the presence of the cancer-associated antigen in the sample. Optionally, the sample may contain cells (which may be cancer cells) suspected of expressing the cancer-associated antigen. The antibody employed in the method may optionally be detectably labeled, attached to a solid support, or the like.

A further embodiment of the present invention is directed to a method of diagnosing the presence of a tumor in a mammal, wherein the method comprises detecting the level of expression of a cancer-associated antigen (a) in a test sample of tissue cells obtained from said mammal, and (b) in a control sample of known normal non-cancerous cells of the same tissue origin or type, wherein a higher level of expression of the cancer-associated antigen in the test sample, as compared to the control sample, is indicative of the presence of tumor in the mammal from which the test sample was obtained.

Another embodiment of the present invention is directed to a method of diagnosing the presence of a tumor in a mammal, wherein the method comprises (a) contacting a test sample comprising tissue cells obtained from the mammal with an antibody that binds to a cancer-associated antigen as described herein (such as an antibody to MUC5AC, CEACAM5, CEACAM6, or the A33 antigen) and (b) detecting the formation of a complex between the antibody and the cancer-associated antigen in the test sample, wherein the formation of a complex is indicative of the presence of a tumor in the mammal. Optionally, the antibody employed is detectably labeled, attached to a solid support, or the like, and/or the test sample of tissue cells is obtained from an individual suspected of having a cancerous tumor.

Yet another embodiment of the present invention is directed to a method of binding an antibody to a cell that expresses a cancer-associated antigen as described herein (such as MUC5AC, CEACAM5, CEACAM6, or the A33 antigen), wherein the method comprises contacting a cell that expresses a cancer-associated antigen with said antibody under conditions which are suitable for binding of the antibody to said cancer-associated antigen and allowing binding therebetween. In preferred embodiments, the antibody is labeled with a molecule or compound that is useful for qualitatively and/or quantitatively determining the location and/or amount of binding of the antibody to the cell.

Except where otherwise provided the techniques and procedures are generally performed according to conventional methods well known in the art and as described in various general and more specific references that are cited and discussed throughout the present specification. See, e.g., Sambrook, et al (2001) Molecular Cloning: A Laboratory Manual [3rd Ed] Cold Spring Harbor Laboratory Press. Standard techniques may be used for recombinant DNA, oligonucleotide synthesis, and tissue culture, and transformation (e.g., electroporation, lipofection). Enzymatic reactions and purification techniques may be performed according to manufacturer's specifications or as commonly accomplished in the art or as described herein. The nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

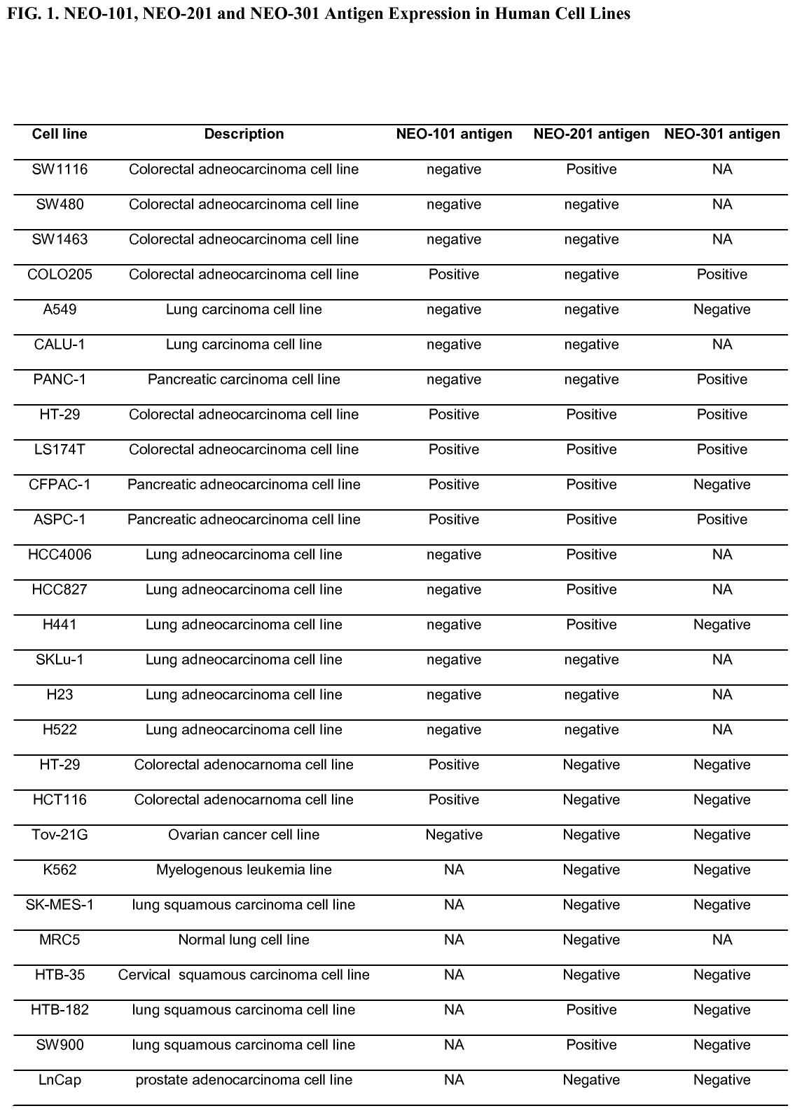

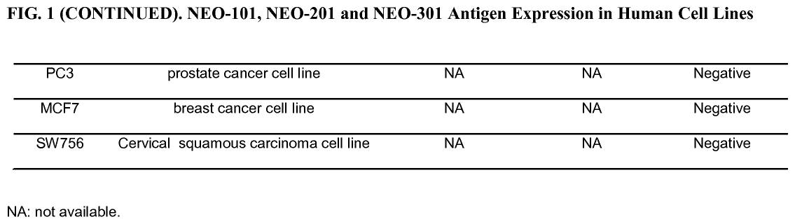

It is a further object of the invention to provide a method of treating a cancer type corresponding to any of the human cell lines comprised in Table 2 and FIG. 1, or any cancer comprised in Table 2 and FIG. 1 other than colon, pancreatic or lung cancer, by the administration of a therapeutically effective amount of an antibody having the epitopic specificity of NEO-102, NEO-201 or NEO-301 or a fragment thereof and one or more agents that target an apoptotic pathway (such as the common, extrinsic or intrinsic apoptotic pathway).

It is another object of the invention to provide a method of treating a cancer corresponding to any of the human cell lines comprised in Table 2 and FIG. 1, or any cancer comprised in Table 2 and FIG. 1 other than colon, pancreatic or lung cancer, by the administration of a therapeutically effective amount of an antibody that competes with NEO-102, NEO-201 or NEO-301 or a fragment thereof and one or more agents that target an apoptotic pathway (such as the common, extrinsic or intrinsic apoptotic pathway).

It is also an object of the invention to provide a method of detecting a cancer, such as breast, ovarian, cervical, or uterine cancer, by the administration of a diagnostically effective amount of an antibody having the epitopic specificity of NEO-102, NEO-201 or NEO-301 or a fragment thereof.

It is also an object of the invention to provide a method of detecting a cancer corresponding to any of the human cell lines comprised in Table 2 and FIG. 1, or any cancer comprised in Table 2 and FIG. 1 other than colon, pancreatic or lung cancer, by the administration of a diagnostically effective amount of an antibody that competes with NEO-102, NEO-201 or NEO-301 or a fragment thereof.

It is another object of the invention to provide a method for diagnosing cancer in a subject by detecting NEO-102, NEO-201 or NEO-301 antigen-bearing cancer cells which comprises: (a) obtaining a sample from a subject suspected of having cancer; (b) contacting the sample with NEO-102, NEO-201 or NEO-301 or a fragment of such antibody, which is detectably labeled, under appropriate conditions so as to produce an antibody-antigen complex or a fragment-antigen complex comprising the detectably labeled antibody or fragment bound to any tumor cells expressing NEO-102, NEO-201 or NEO-301 antigen in the sample; (c) removing any labeled antibody or fragment not in the antibody antigen complex or fragment-antigen complex formed in step (b); and (d) determining the presence of any antibody antigen complex or any fragment-antigen complex by detecting the detectably labeled antibody or Fab fragment, the presence of antibody antigen complex or Fab fragment-antigen complex being diagnostic of cancer so as to thereby diagnose cancer in the subject, preferably wherein the detectably labeled antibody or Fab fragment is labeled with a radioactive isotope, an enzyme, a dye, a biotin, a fluorescent label or a chemiluminescent label.

It is also an object of the invention to provide a method for monitoring progression of a cancer, wherein the cancer is one that comprises cells that express at least one tumor-associated antigenic epitope specifically bound by at least one of the NEO-102, NEO-201 or NEO-301 antibodies or fragments thereof in a subject, comprising: a) administering to a subject diagnosed with cancer that comprises cells that express at least one tumor-associated antigenic epitope specifically bound by at least one of the NEO-102, NEO-201 or NEO-301 antibodies, an antibody or fragment directed to an epitope specifically bound by at least one of NEO-102, NEO-201 or NEO-301 or a fragment of such an antibody, and wherein the antibody or fragment is detectably labeled, under appropriate conditions so as to bind the antibody or the fragment to NEO-101, NEO-201 or NEO-301 antigen bearing cells in the subject; b) determining the presence of detectably labeled antibody or fragment bound to the surface of cells in the subject; c) comparing the presence of the detectably labeled antibody or fragment bound to cells in step (b) with the presence of detectably labeled antibody bound to cells at (i) diagnosis time or (ii) after treatment, wherein a greater presence of detectably labeled antibody or fragment bound to cells in step (b) than at (i) diagnosis time or (ii) after treatment, indicates progression of the cancer in the subject and a lesser presence of detectably labeled antibody or fragment bound to cells in step (b) than at (i) diagnosis time or (ii) after treatment indicates regression of the cancer in the subject.

It is another object of the invention to provide a method of monitoring the efficacy of a cancer therapy, wherein the cancer comprises cells that express or overexpress a tumor-associated antigenic epitope specifically bound by at least one of the NEO-102, NEO-201 or NEO-301 antibodies or a fragment thereof, comprising contacting a subject receiving said therapy with an antibody or antibody fragment that specifically binds a tumor-associated antigenic epitope specifically bound by at least one of the NEO-101, NEO-201 or NEO-301 antibodies and determining the amount of antigen expression in said subject wherein a decrease in antigen expression indicates that said therapy is effective at treating cancer in said subject.

In this application is described the use of NEO-102, NEO-201 and NEO-301 in flow cytometry experiments in order to identify additional human cancer cell lines that express the target antigen bound by these respective antibodies. These results confirm the utility of these antibodies in naked or conjugated form for treating and detecting different human cancers which correspond to these specific cell lines. It is anticipated that these studies will confirm that these antibodies may be used to treat or detect all stages of cancer wherein the cancer cell or precancerous cells express these antigens, e.g. pre-cancer and Stage I, II, II and IV cancers including metastatic cancers that express these target antigens.

Representative cancer cell lines and different human cancers that potentially may be evaluated for treatment or detection using the subject antibodies are set forth in comprised in Table 2 and FIG. 1. These cancers include by way of example colon, pancreatic, lung, prostate, melanoma, breast, melanoma, ovarian, uterine, and cervical cancers, and mesothelioma.

In addition, the present invention includes the production of radiolabeled forms of each of these antibodies and the use of the resultant radioimmunoconjugates in biodistribution studies effected animal models (e.g., in mice with human tumor xenografts) as well as human patients, in order to detect cancer cells that express a target antigen bound by NEO-102, NEO-201 and/or NEO-301. In the exemplary described embodiment the radio label used is indium-III, however, other radiolabels may alternatively be used including those described in PCT/US2011/041502, by Bristol et al, incorporated by reference herein.

These studies corroborate that NEO-102, NEO-201 and NEO-301, in labeled and unlabeled form, have broad application in the detection and treatment of different types of human cancer and should be substantially non-toxic to non-target normal tissues and organs. Said cancers include without limitation thereto breast, ovarian, cervical, or uterine cancer.

### Diagnostic Methods

The NPC-1, 16C3, and A33 antigens, antibody which selectively bind the NPC-1, 16C3, or A33 antigen, and antigen-binding fragments thereof may be used in diagnostic methods for detecting the presence or absence of an NPC-1, 16C3, or A33 antigen. The NPC-1, 16C3, and A33 antigens, antibody which selectively bind the NPC-1, 16C3, or A33 antigen, and antigen-binding fragments thereof, A33 antigen may be used in methods comprising (a) contacting a test sample with an antibody, or fragment thereof, that binds a NPC-1 epitope, 16C3 epitope, and/or A33 antigen, and (b) assaying for antibody-epitope complexes, wherein the presence of said epitope is indicative of a carcinoma. Further, the NPC-1, 16C3, and A33 antigens, antibody which selectively bind a NPC-1, 16C3, or A33 antigen, and antigen-binding fragments, may be used in a method for detecting the presence of a NPC-1 epitope, 16C3 epitope, and/or A33 antigen in a patient comprising (a) administering to said patient a labeled monoclonal antibody, or fragment thereof, that binds a NPC-1 epitope, 16C3 epitope, and/or A33 antigen and (b) detecting the presence of a NPC-1 epitope, 16C3 epitope, and/or A33 antigen; wherein the presence of said epitope is indicative of a carcinoma. The antibody-epitope complex may be detected by Western blot, radioimmunoassay, ELISA (enzyme linked immunosorbent assay), “sandwich” immunoassay, immunoprecipitation assay, precipitation reaction, gel diffusion precipitation reaction, immunodiffusion assay, agglutination assay, complement-fixation assay, immunohistochemical assay, fluorescent immunoassay, and protein A immunoassay. The sample may be sample is a tissue biopsy, lymph, urine, cerebrospinal fluid, amniotic fluid, inflammatory exudate, blood, serum, stool, or liquid collected from the colorectal tract.

The NPC-1, 16C3, and A33 antigens, antibody which selectively bind a NPC-1, 16C3, or A33 antigen, and antigen-binding fragments thereof may be used in diagnostic methods for detecting the presence or absence of an NPC-1, 16C3, or A33 antigen, wherein the presence of the antigen is indicative of cancer including but not limited to lung, breast, ovarian, cervical, uterine cancer, pancreas, esophageal, colorectal, or liver cancer. The diagnostic methods may be used with patients at risk of cancer or patients without symptoms.

The antibodies which selectively bind a NPC-1, 16C3, or A33 antigen may be recombinant. The fragments of antibodies which selectively bind a NPC-1, 16C3, or A33 antigen may be a Fab, Fab′, F(ab′)2, Fv, CDR, paratope, or portion of an antibody that is capable of binding the antigen. The antibodies which selectively bind a NPC-1, 16C3, or A33 antigen may be chimeric, humanized, anti-idiotypic, single-chain, bifunctional, or co-specific. The antibodies which selectively bind a NPC-1, 16C3, or A33 antigen may be or fragment is conjugated to a label, including but not limited to a chemiluminescent label, paramagnetic label (e.g., aluminum, manganese, platinum, oxygen, lanthanum, lutetium, scandium, yttrium, or gallium), an MRI contrast agent, fluorescent label, bioluminescent label, or radioactive label.

Additionally, NPC-1, 16C3, and A33 antigens, antibody which selectively bind a NPC-1, 16C3, or A33 antigen, and antigen-binding fragments thereof, may be attached to a solid support (e.g., bead, test tube, sheet, culture dish, or test strip) such as an array.

The method may detect colorectal polyps. The method may further comprise additional testing for the presence of tumors including but not limited to benign tumors, malignant tumors, metastatic tumors, and non-metastatic tumors. For example, the diagnostic method may detect pre-cancerous cells that express a cell marker comprising a NPC-1 epitope, 16C3 epitope, and/or A33 antigen.

The method may comprise imaging a NPC-1 epitope, 16C3 epitope, and/or A33 antigen by positron emission tomography (PET), CCD low-light monitoring system, x-ray, CT scanning, scintigraphy, photo acoustic imaging, single photon emission computed tomography (SPECT), magnetic resonance imaging (MRI), ultrasound, paramagnetic imaging, and endoscopic optical coherence tomography.

The invention also provides a method for genetic diagnosis of a risk for cancer comprising taking a nucleic acid sample from a patient, analyzing said nucleic acid comprising comparing to cancer specific MUC5AC, CEACAM5, CEACAM6, or A33 sequence, wherein if the patient's nucleic acid sample matches the cancer specific MUC5AC, CEACAM5, CEACAM6, or A33 sequence, the patient is at risk for developing cancer.

The NPC-1, 16C3, and A33 antigens may be used as a cancer biomarker. Detection of the NPC-1, 16C3, or A33 antigens in a biological sample, such as a subject's serum, biopsied neoplastic cells or fecal sample, may be performed by means of the anti-NPC-1, anti-16C3, or anti-A33 antigen antibody. For example, a biological sample (e.g., a tumor, serum or fecal sample) is obtained from a subject, then NPC-1, 16C3, or A33 antigen is measured (e.g., by ELISA or PCR), and compared with corresponding samples from normal subjects. Measuring methods include any method of nucleic acid detection, for example in situ hybridization using antisense NPC-1, 16C3, or A33 antigen DNA or cRNA oligonucleotide probes, ultra-high throughput sequencing, nanostring technology, microarrays, rolling circle amplification, proximity-mediated ligation, PCR, qRT-PCR ChIP, ChIP-qPCR, or NPC-1, 16C3, or A33 antigen-binding antibodies. Comparatively high levels of NPC-1, 16C3, and A33 antigens indicate the presence and/or severity of pancreas or colon cancer, and may indicate metastasis or poor cancer prognosis.

The NPC-1, 16C3, and A33 antigens, antibody which selectively bind a NPC-1, 16C3, or A33 antigen, and antigen-binding fragments thereof, may be used in SQUID (Superconducting Quantum Interference Device) techniques for diagnostic methods. The SQUID technique comprises attaching nanoparticles of iron oxide to antibodies, which are then injected into the patient. If a tumor is present, the antibodies with conjugated nanoparticles recognize and bind to the NPC-1, 16C3, or A33 antigen on tumor cells. See, e.g., Hao, et al. (2010) Journal of Physics 43: 474004. In a SQUID method, the patient is then surrounded with sensitive magnetic coils in a superconducting quantum interference device (SQUID). A magnetic field is generated and all of the metal nanoparticles align in one direction. When the magnetic field is broken, the nanoparticles emit an electromagnetic signal as they relax back into their original state. By measuring the strength of the signal, on emay tell how many metal particles, and therefore how many tumor cells, may be present, and where in the patient the tumor cells are located. See, e.g., Shao, et al. (2010) Beilstein Journal of Nanotechnology 1: 142-154.

### Samples and Procurement of Samples

The samples used in the methods described herein may be taken from a subject (patient) include but are not limited to a body fluid or secretion including but not limited to blood, serum, urine, plasma, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system and lavage of any other part of the body or system in the body; samples of any organ including isolated cell(s) or tissue(s), wherein the cell or tissue can be obtained from an organ selected from, but not limited to lung, colon, ovarian, uterine, cervical, and/or breast tissue; stool or a tissue sample, or any combination thereof. In some embodiments, the term encompasses samples of in vivo cell culture constituents. Prior to be subjected to the diagnostic assay, the sample can optionally be diluted with a suitable diluent.

Numerous well known tissue or fluid collection methods can be utilized to collect the biological sample from the subject in order to determine the level of DNA, RNA and/or polypeptide of the marker of interest in the subject. Examples of tissue or fluid collection methods include, but are not limited to, fine needle biopsy, needle biopsy, core needle biopsy and surgical biopsy (e.g., brain biopsy), and lavage. Regardless of the procedure employed, once a biopsy/sample is obtained the level of the marker may be determined and a diagnosis can thus be made.

### Detection of NPC-1, 16C3, A33 Antigens

The invention also provides a method for detecting the NPC-1, 16C3, and A33 antigens of this invention in a biological sample, comprising: contacting a biological sample with an antibody specifically recognizing a NPC-1, 16C3, or A33 antigen according to the present invention and detecting said interaction; wherein the presence of an interaction correlates with the presence of a NPC-1, 16C3, or A33 antigen in the biological sample.

The NPC-1, 16C3, and A33 antigens described herein are non-limiting examples of markers for diagnosing a disease and/or an indicative condition. Each marker of the present invention may be used alone or in combination, for various uses, including but not limited to, prognosis, prediction, screening, early diagnosis, determination of progression, therapy selection and treatment monitoring of a cancer (e.g., breast, ovarian, cervical, or uterine cancer).

The cancers that may be detected using the methods described herein include but are not limited to non-solid and solid tumors, cancer of the breast, prostate, lung, ovary, colon, uterus, stomach, cervix, liver, pancreas, and wherein the cancer may be invasive or metastatic.

Each NPC-1, 16C3, and A33 antigens of the present invention may be used alone or in combination, for various uses, including but not limited to, prognosis, prediction, screening, early diagnosis, determination of progression, therapy selection and treatment monitoring of cancers such as non-solid and solid tumors, cancer of the breast, prostate, lung, ovary, colon, uterus, stomach, cervix, liver, pancreas, and wherein the cancer may be invasive or metastatic. Such a combination may optionally comprise any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker.

Markers of the present invention may optionally be used alone or in combination with known markers for lung cancer, including but not limited to CEA, CA15-3, beta-2-microglobulin, CA19-9, TPA, and/or in combination with the known proteins for the variant marker as described herein.

Markers of the present invention might optionally be used alone or in combination with known markers for ovarian cancer, including but not limited to CEA, CA125 (Mucin 16), CA72-4TAG, CA-50, CA 54-61, CA-195 and CA 19-9 in combination with CA-125, and/or in combination with the known proteins for the variant marker as described herein.

Markers of the present invention might optionally be used alone or in combination with known markers for colon cancer, including but not limited to CEA, CA19-9, CA50, and/or in combination with the known proteins for the variant marker as described herein.

Typically the level of the marker in a biological sample obtained from the subject is different (i.e., increased or decreased) from the level of the same marker in a similar sample obtained from a healthy individual (examples of biological samples are described herein).

Determining the level of the same marker in normal tissues of the same origin may be effected along-side to detect an elevated expression and/or amplification and/or a decreased expression, of the marker as opposed to the normal tissues.

The present invention also provides methods, uses, devices and assays for the diagnosis of cancers such as non-solid and solid tumors, cancer of the breast, prostate, lung, ovary, colon, uterus, stomach, cervix, liver, pancreas, and wherein the cancer may be invasive or metastatic. Optionally a plurality of markers may be used with the present invention. The plurality of markers may optionally include a markers described herein, and/or one or more known markers. The plurality of markers is preferably then correlated with the disease or condition. For example, such correlation may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level. Also, such correlating may optionally comprise determining a temporal change in at least one of the markers, and wherein the temporal change is used in the correlating step.

Such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Correlating may optionally comprise determining whether a ratio of marker concentrations for two markers is outside a range and/or above or below a threshold. Optionally, if the ratio is above or below the threshold level and/or outside a range, the ratio correlates with the disease or condition. Optionally, a combination of two or more these correlations may be used with a single panel and/or for correlating between a plurality of panels. Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, may be combined in a single assay or device. Markers may also be commonly used for multiple purposes by, for example, applying a different threshold or a different weighting factor to the marker for the different purpose(s).

The panels may comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those cancers such as non-solid and solid tumors, cancer of the breast, prostate, lung, ovary, colon, uterus, stomach, cervix, liver, pancreas, and wherein the cancer may be invasive or metastatic that may feature one or more similar or identical symptoms.

One or more diagnostic or prognostic indicators are correlated to a condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment.

NPC-1, 16C3, or A33 antigens may be featured as a biomarker for detecting cancers such as non-solid and solid tumors, cancer of the breast, prostate, lung, ovary, colon, uterus, stomach, cervix, liver, pancreas, and wherein the cancer may be invasive or metastatic.

The present invention optionally and preferably encompasses any amino acid sequence or fragment thereof encoded by a nucleic acid sequence corresponding to NPC-1, 16C3, or A33 antigens as described herein. Any oligopeptide or peptide relating to such an amino acid sequence or fragment thereof may optionally also (additionally or alternatively) be used as a biomarker.

The present invention provides a method for detecting a polynucleotide of this invention in a biological sample, using NAT based assays, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of the polynucleotide in the biological sample. Non-limiting examples of methods or assays are described herein. The present invention also relates to kits based upon such diagnostic methods or assays.

Additionally, the NPC-1, 16C3, and A33 antigens may be used as specific biomarkers for pancreas and colon cancer, and can be measured in biopsied tissue as well as in subject serum and fecal samples, as described herein. Additionally, diagnostic procedures used to detect colorectal cancer including but not limited to fecal occult blood test (FOBT), colonoscopy, computed tomographic colonography (virtual colonoscopy) [detects colorectal lesions larger than 6 mm in diameter with the same sensitivity as colonoscopy], flexible sigmoidoscopy, double-contrast barium enema, and digital rectal examination. Winawer, et al. (1997) Am J. Gastoenterology 112: 594-642; Blum (1995) Eur. J. Canc. 31: 1369-72; Ransohoff & Sandler (2002) N. Engl. J. Med. 346: 346 11; Bruzzi (2002) N. Engl. J. Med. 346: 1672-74; and Laghi, et al. (2002) Am. J. Surg. 183: 124-31.

### Immunoassays

The NPC-1, 16C3, or A33 antigens, antibodies and antigen-binding fragments that bind the NPC-1, 16C3, or A33 antigen, may be used in immunoassays to qualitatively or quantitatively detect and analyze markers in a sample. This method comprises providing an antibody specifically binds to a NPC-1, 16C3, and/or A33 antigen; contacting a sample with the antibody; and detecting the presence of a complex of the antibody bound to the marker in the sample.

An NPC-1, 16C3, and/or A33 antigen may be detected and/or quantified using any of a number of well recognized immunological binding assays. Useful assays include, for example, an enzyme immune assay (EIA) such as enzyme-linked immunosorbent assay (ELISA), a radioimmunoassay (RIA), a Western blot assay, or a slot blot assay. See, e.g., U.S. Pat. Nos. 4,366,241; 4,376,110; 4,517,288; and 4,837,168. Generally, a sample obtained from a subject can be contacted with the antibody specifically binds the NPC-1, 16C3, and/or A33 antigen.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies may be attached to a solid support.

After incubating the sample with antibodies, the mixture is washed and the antibody-marker complex formed may be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations. Usually the assays will be carried out at ambient temperature, although they can be conducted over a range of temperatures (e.g., 10 degrees C.-40 degrees C.).

The immunoassay can be used to determine a test amount of a marker in a sample from a subject. First, a test amount of a marker in a sample may be detected using the immunoassay methods described above. If a marker is present in the sample, it will form an antibody-marker complex with an antibody specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal. Several immunoassays are known in the art and the NPC-1, 16C3, and/or A33 antigens, and antibodies specific for said antigens described herein may used in such immunoassays including but not limited to radio-immunoassay (RIA), enzyme linked immunosorbent assay (ELISA), magnetic immunoassay, immunoblot, Western blot, immunoprecipitation assays, immunohistochemical analysis, and fluorescence activated cell sorting (FACS). See Wild, (2008) [Ed.] The Immunoassay Handbook [3rd Ed.] Elsevier.

### Radio-Imaging Methods

The NPC-1, 16C3, or A33 antigens, antibodies and antigen-binding fragments that bind the NPC-1, 16C3, or A33 antigen, may be used in radio-imaging methods to diagnosis cancer including pancreatic and colorectal cancer, or monitor the progression of tumors. These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. SPECT may optionally be used with two labels simultaneously. See U.S. Pat. No. 6,696,686.

### Definitions

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting.

As used in the description herein and throughout the claims that follow, the meaning of “a,” “an,” and “the” includes plural reference unless the context clearly dictates otherwise.

“Adjuvant,” as used herein, refers broadly to any substance which is incorporated into or administered simultaneously with NPC-1 epitope peptidomimetic of the invention which potentiates the immune response in the subject. Adjuvants include but are not limited to aluminum compounds, e.g., gels, aluminum hydroxide and aluminum phosphate, and Freund's complete or incomplete adjuvant (e.g., in which the PS/A antigen is incorporated in the aqueous phase of a stabilized water in paraffin oil emulsion). The paraffin oil may be replaced with different types of oils, e.g., squalene or peanut oil. Other materials with adjuvant properties, include BCG (attenuated Mycobacterium tuberculosis), calcium phosphate, levamisole, isoprinosine, polyanions (e.g., poly A:U), lentinan, pertussis toxin, lipid A, saponins, QS-21 and peptides, e.g. muramyl dipeptide. Rare earth salts, e.g., lanthanum and cerium, may also be used as adjuvants. The amount of adjuvants depends on the subject and the particular antigen used and can be readily determined by one skilled in the art without undue experimentation.

“Amino acid,” as used herein, refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Antibody,” as used herein, refers broadly to any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, from all sources, e.g., human, rodent, rabbit, cow, sheep, pig, dog, chicken, are considered to be “antibodies.” Antibodies include but are not limited to chimeric antibodies, human antibodies and other non-human mammalian antibodies, humanized antibodies, single chain antibodies (scFvs), camelbodies, nanobodies, IgNAR (single-chain antibodies derived from sharks), small-modular immunopharmaceuticals (SMIPs), and antibody fragments (e.g., Fabs, Fab′, F(ab′)₂.) Numerous antibody coding sequences have been described; and others may be raised by methods well-known in the art. See Streltsov, et al. (2005) Protein Sci. 14(11): 2901-9; Greenberg, et al. (1995) Nature 374(6518): 168-173; Nuttall, et al. (2001) Mol Immunol. 38(4): 313-26; Hamers-Casterman, et al. (193) Nature 363(6428): 446-8; Gill, et al. (2006) Curr Opin Biotechnol. 17(6): 653-8.

The term “antibody” is used in the broadest sense and specifically covers, for example, single monoclonal antibodies (including agonist, antagonist, and neutralizing antibodies), antibody compositions with polyepitopic specificity, polyclonal antibodies, single chain antibodies, and fragments of antibodies as long as they exhibit the desired biological or immunological activity. The term “immunoglobulin” (Ig) is used interchangeable with antibody herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the alpha and gamma chains and four CH domains for mu and epsilon isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha, delta, epsilon, gamma, and mu, respectively. The gamma and alpha classes are further divided into subclasses on the basis of relatively minor differences in CH sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a CL and at least heavy chain constant domains, CH1, CH2 and CH3. The constant domains may be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1E10⁻⁷ M, preferably no more than about 1E10⁻⁸ and most preferably no more than about 1E10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence.

Additionally, when referring to a modified Fc domain or “Fc variant”, the terms “Kabat numbering system,” “Kabat position,” “Kabat residue,” “Kabat number” or the like, or in any instance in which an Fc modification is identified by number without reference to a specific numbering system (e.g., “position” followed by a number), refer to positions numbered according to the EU index or EU numbering scheme (Kabat et al., 1991, Sequences of Proteins of Immunological Interest, 5th Ed., United States Public Health Service, National Institutes of Health, Bethesda, incorporated by reference). The terms “EU index” or “EU index as in Kabat” and the like refer to the numbering of the EU antibody (Edelman et al., 1969, Proc Natl Acad Sci USA 63:78-85, incorporated by reference). Additionally, except where stated otherwise, when referring to an Fc variant relative terms (such as “increased” or “decreased”) refer to the change in that attribute relative to the same Fc variant (or polypeptide containing said Fc variant) without said modification.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. Exemplary antibodies of the present disclosure may include one or more modifications that decrease one or more effector functions, such as alterations in the amino acid sequence, or alterations in the location, extent, or type of glycosylation.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Antigen,” as used herein, refers broadly to a molecule or a portion of a molecule capable of being bound by an antibody which is additionally capable of inducing an animal to produce an antibody capable of binding to an epitope of that antigen. An antigen may have one epitope, or have more than one epitope. The specific reaction referred to herein indicates that the antigen will react, in a highly selective manner, with its corresponding antibody and not with the multitude of other antibodies which may be evoked by other antigens. Antigens may be tumor specific (e.g., expressed by neoplastic cells of pancreatic and colon carcinoma.)

“Antigenic composition,” as used herein, refers broadly to a composition that elicits an immune response.

“Cancer,” as used herein, refers broadly to any neoplastic disease (whether invasive or metastatic) characterized by abnormal and uncontrolled cell division causing malignant growth or tumor.

“Chimeric antibody,” as used herein, refers broadly to an antibody molecule in which the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug; or the variable region, or a portion thereof, is altered, replaced or exchanged with a variable region having a different or altered antigen specificity.

“Conservatively modified variants,” as used herein, applies to both amino acid and nucleic acid sequences, and with respect to particular nucleic acid sequences, refers broadly to conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) may be modified to yield a functionally identical molecule.

“Complementarity determining region,” “hypervariable region,” or “CDR,” as used herein, refers broadly to one or more of the hyper-variable or complementarily determining regions (CDRs) found in the variable regions of light or heavy chains of an antibody. See Kabat, et al. (1987) “Sequences of Proteins of Immunological Interest” National Institutes of Health, Bethesda, MD. These expressions include the hypervariable regions as defined by Kabat, et al (1991) “Sequences of Proteins of Immunological Interest” U.S. Dept. of Health and Human Services, or the hypervariable loops in 3-dimensional structures of antibodies. Chothia and Lesk (1987) J Mol. Biol. 196: 901-17. The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction. Kashmiri (2005) Methods 36: 25-34.

“Control amount,” as used herein, refers broadly to a marker can be any amount or a range of amounts to be compared against a test amount of a marker. For example, a control amount of a marker may be the amount of a marker in a patient with a particular disease or condition or a person without such a disease or condition. A control amount can be either in absolute amount (e.g., microgram/ml) or a relative amount (e.g., relative intensity of signals).

“Differentially present,” as used herein, refers broadly to differences in the quantity or quality of a marker present in a sample taken from patients having a disease or condition as compared to a comparable sample taken from patients who do not have one of the diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker may be considered to be differentially present. Optionally, a relatively low amount of up-regulation may serve as the marker.

“Diagnostic,” as used herein, refers broadly to identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Diagnosing,” as used herein, refers broadly to classifying a disease or a symptom, determining a severity of the disease, monitoring disease progression, forecasting an outcome of a disease and/or prospects of recovery. The term “detecting” may also optionally encompass any of the foregoing. Diagnosis of a disease according to the present invention may, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

An “effective amount” of a composition such as a polypeptide, drug, siRNA or analog thereof, antibody, pharmaceutical, small molecule, or other compound, or an agonist or antagonist thereof, is an amount sufficient to carry out a specifically stated purpose. An “effective amount” may be determined empirically and in a routine manner, in relation to the stated purpose.

“Expression vector,” as used herein, refers broadly to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

“Framework region” or “FR,” as used herein, refers broadly to one or more of the framework regions within the variable regions of the light and heavy chains of an antibody. See Kabat, et al (1987) “Sequences of Proteins of Immunological Interest,” National Institutes of Health, Bethesda, MD. These expressions include those amino acid sequence regions interposed between the CDRs within the variable regions of the light and heavy chains of an antibody.

“Heterologous,” as used herein, refers broadly to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“High affinity,” as used herein, refers broadly to an antibody having a dissociation constant of about or less than 10⁻⁸ M, more preferably about or less than 10⁻⁹ M and even more preferably about or less than 10⁻¹⁰ M for a target antigen. However, “high affinity” binding can vary for other antibody isotypes. For example, “high affinity” binding for an IgM isotype refers to an antibody having a dissociation constant of about or less than 10⁻⁷ M and even more preferably about or less than 10⁻⁸ M for a target antigen.

“Homology,” as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison. The term “sequence identity” may be used interchangeably with “homology.”

“Host cell,” as used herein, refers broadly to a cell that contains an expression vector and supports the replication or expression of the expression vector. Host cells may be prokaryotic cells such as E. coli, or eukaryotic cells such as yeast, insect (e.g., SF9), amphibian, or mammalian cells such as CHO, HeLa, HEK-293, e.g., cultured cells, explants, and cells in vitro.

“Hybridization,” as used herein, refers broadly to the physical interaction of complementary (including partially complementary) polynucleotide strands by the formation of hydrogen bonds between complementary nucleotides when the strands are arranged antiparallel to each other.

Administration “in combination with” one or more further therapeutic agents includes simultaNEOus (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

“K-assoc” or “Ka”, as used herein, refers broadly to the association rate of a particular antibody-antigen interaction, whereas the term “Kdiss” or “Kd,” as used herein, refers to the dissociation rate of a particular antibody-antigen interaction. The term “KD”, as used herein, is intended to refer to the dissociation constant, which is obtained from the ratio of Kd to Ka (i.e., Kd/Ka) and is expressed as a molar concentration (M). KD values for antibodies can be determined using methods well established in the art.

“Immunoassay,” as used herein, refers broadly to an assay that uses an antibody to specifically bind an antigen. The immunoassay may be characterized by the use of specific binding properties of a particular antibody to isolate, target, and/or quantify the antigen.

“Isolated,” as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated antibody”).

“Label” or a “detectable moiety” as used herein, refers broadly to a composition detectable by spectroscopic, photochemical, biochemical, immunochemical, chemical, or other physical means.

“Low stringency,” “medium stringency,” “high stringency,” or “very high stringency conditions,” as used herein, refers broadly to conditions for nucleic acid hybridization and washing. Guidance for performing hybridization reactions can be found in Ausubel, et al. (2002) Short Protocols in Molecular Biology (5^{th} Ed.) John Wiley & Sons, NY. Exemplary specific hybridization conditions include but are not limited to: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C.

“Mammal,” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, gerbils, goats, gorillas, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington DC.

“Nucleic acid” or “nucleic acid sequence,” as used herein, refers broadly to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Paratope,” as used herein, refers broadly to the part of an antibody which recognizes an antigen (e.g., the antigen-binding site of an antibody.) Paratopes may be a small region (e.g., 15-22 amino acids) of the antibody's Fv region and may contain parts of the antibody's heavy and light chains. See Goldsby, et al. Antigens (Chapter 3) Immunology (5^{th} Ed.) New York: W.H. Freeman and Company, pages 57-75.

“Patient,” as used herein, refers broadly to any animal who is in need of treatment either to alleviate a disease state or to prevent the occurrence or reoccurrence of a disease state. Also, “Patient” as used herein, refers broadly to any animal who has risk factors, a history of disease, susceptibility, symptoms, signs, was previously diagnosed, is at risk for, or is a member of a patient population for a disease. The patient may be a clinical patient such as a human or a veterinary patient such as a companion, domesticated, livestock, exotic, or zoo animal. The term “subject” may be used interchangeably with the term “patient”.

“Peptidomimetic,” as used herein refers broadly to a compound that can imitate or block the biological effect of a peptide on a molecular level. Peptidomimetics may be polymers designed to mimic a peptide, such as peptoids and β-peptides, or may be a peptide that mimics a different peptide.

“Polypeptide,” “peptide” and “protein,” are used interchangeably and refer broadly to a polymer of amino acid residues. The terms apply to amino acid polymers in which one or more amino acid residue is an analog or mimetic of a corresponding naturally occurring amino acid, as well as to naturally occurring amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” include glycoproteins, as well as non-glycoproteins.

“Promoter,” as used herein, refers broadly to an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

“Prophylactically effective amount,” as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

“Prophylaxis,” as used herein, refers broadly to a course of therapy where signs and/or symptoms are not present in the patient, are in remission, or were previously present in a patient. Prophylaxis includes preventing disease occurring subsequent to treatment of a disease in a patient. Further, prevention includes treating patients who may potentially develop the disease, especially patients who are susceptible to the disease (e.g., members of a patent population, those with risk factors, or at risk for developing the disease).

“Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Specifically (or selectively) binds” to an antibody or “specifically (or selectively) immunoreactive with,” or “specifically interacts or binds,” as used herein, refers broadly to a protein or peptide (or other epitope), refers, in some embodiments, to a binding reaction that is determinative of the presence of the protein in a heterogeNEOus population of proteins and other biologies. For example, under designated immunoassay conditions, the specified antibodies bind to a particular protein at least two times greater than the background (non-specific signal) and do not substantially bind in a significant amount to other proteins present in the sample. Typically a specific or selective reaction will be at least twice background signal or noise and more typically more than about 10 to 100 times background.

“Specifically hybridizable” and “complementary” as used herein, refer broadly to a nucleic acid can form hydrogen bond(s) with another nucleic acid sequence by either traditional Watson-Crick or other non-traditional types. The binding free energy for a nucleic acid molecule with its complementary sequence is sufficient to allow the relevant function of the nucleic acid to proceed, e.g., RNAi activity. Determination of binding free energies for nucleic acid molecules is well known in the art. See, e.g., Turner, et al. (1 87) CSH Syrnp. Quant. Biol. LII: 123-33; Frier, et al (1986) PNAS 83: 9373-77; Turner, et al. (1987) J. Am. Chem. Soc. 109: 3783-85. A percent complementarity indicates the percentage of contiguous residues in a nucleic acid molecule that can form hydrogen bonds (e.g., Watson-Crick base pairing) with a second nucleic acid sequence (e.g., about at least 5, 6, 7, 8, 9, 10 out of 10 being about at least 50%, 60%, 70%, 80%, 90%, and 100% complementary, inclusive). “Perfectly complementary” or 100% complementarity refers broadly all of the contiguous residues of a nucleic acid sequence hydrogen bonding with the same number of contiguous residues in a second nucleic acid sequence. “Substantial complementarity” refers to polynucleotide strands exhibiting about at least 90% complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically may differ by at least 5 nucleotides.

“Signs” of disease, as used herein, refers broadly to any abnormality indicative of disease, discoverable on examination of the patient; an objective indication of disease, in contrast to a symptom, which is a subjective indication of disease.

“Solid support,” “support,” “substrate,” or “solid phase” as used herein, refer to a non-aqueous matrix to which an antibody or other molecule of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149. The definition broadly includes any material that provides a solid or semi-solid structure with which another material can be attached including but not limited to smooth supports (e.g., metal, glass, plastic, silicon, and ceramic surfaces) as well as textured and porous materials.

“Subjects” as used herein, refers broadly to anyone suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g., rats and mice), lagomorphs, primates, and humans. Any mammalian subject in need of being treated according to the present invention is suitable. Human subjects of both genders and at any stage of development (i.e., NEOnate, infant, juvenile, adolescent, adult) can be treated according to the present invention. The present invention may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “patients.”

“Symptoms” of disease as used herein, refers broadly to any morbid phenomenon or departure from the normal in structure, function, or sensation, experienced by the patient and indicative of disease.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a cancer if, after receiving a therapeutic amount of an antibody (or other drug), the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the antibody may prevent growth and/or kill existing cancer cells, it may be cytostatic and/or cytotoxic. Reduction of these signs or symptoms may also be felt by the patient.

The term “therapeutically effective amount” refers to an amount of an antibody, polypeptide, or other drug effective to “treat” a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

The term “synergistic effect” refers to the result achieved using a combination being greater than the sum of the results that would be achieved using the individual components of the combination. For example, in the context of a treatment method (e.g., treatment of cancer), a synergistic effect indicates that the effect of two or more treatments in combination (e.g., two or more of administration of an antibody, a chemotherapeutic agent, another anti-cancer agent, surgery, or radiation) have an effect that is more than just the additive effect of the individual agents. In an exemplary embodiment, the synergistic effect can be measured with respect to tumor burden, mean survival, or another endpoint as known in the art. For example, without intent to be limited by theory, one treatment (such as radiation or chemotherapy, e.g., gemcitabine) may induced increased expression of a cancer-associated antigen, such as the NPC-1, 16C3, or 31.1 antigen, thereby making the cancer cell more susceptible to antibody binding and/or effector function.

“Variable region” or “VR,” as used herein, refers broadly to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

“Vector,” as used herein, refers broadly to a plasmid, cosmid, phagemid, phage DNA, or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be inserted in order to bring about its replication and cloning. The vector may further contain a marker suitable for use in the identification of cells transformed with the vector.

In order that the invention herein described may be fully understood, the above detailed description is set forth. Various embodiments of the invention are described in detail and may be further illustrated by the provided examples. The examples provided are intended to be illustrative, rather than limiting, on the scope of the invention, which is limited only by the scope of the claims provided below.

### EXAMPLES

### Example 1

This example reports the level of NEO-101 or NEO-102, NEO-201 and NEO-301 Antigen Expression by Human Normal and Cancer Cell Lines. The results indicate that multiple independent samples of different cancers expressed the antigens recognized by NEO-101 or NEO-102, NEO-201 and/or NEO-301.

Antigen expression in cell lines of various origins were detected by Flow Cytometry. Viable cell suspensions were incubated with primary antibody NEO-101, NEO-201 and NEO-301 separately at 10 ug/mL. Human IgG (10 ug/mL) was used as negative control. Goat anti-human IgG (Fc)-phycoerythrin conjugate was used for primary antibody detection; 7-ADD was used for excluding dead cells from analysis. The staining was measured by Flow Cytometry (Guava instrument, Millipore). Median fluorescent intensity (MFI) and % of stained cells was used for determining presence of specific antigen on each cell line.

The results are summarized in FIG. 1. The cell lines tested included breast cancer cells, cervical squamous carcinoma cells, colorectal adenocarcinoma cells, lung adenocarcinoma cells, lung carcinoma cells, lung squamous carcinoma cells, myelogenous leukemia cells, ovarian cancer cells, pancreatic adenocarcinoma cells, pancreatic carcinoma cells, prostate adenocarcinoma cells, and prostate cancer cells. Additionally, normal lung cells were tested.

NEO-101 expression was detected in multiple colorectal adenocarcinoma and multiple pancreatic adenocarcinoma samples. NEO-201 expression was detected in multiple colorectal adenocarcinoma, multiple pancreatic adenocarcinoma, multiple lung adenocarcinoma, and multiple lung squamous cell carcinoma cell lines. NEO-301 expression was detected in colorectal adenocarcinoma and pancreatic adenocarcinoma cell lines.

### Example 2

This example reports prevalence of NEO-101, NEO-201, and NEO-301 antigen expression in cancer cell samples obtained from cancer tissue microarrays and from paraffin tissue sections.

Immunohistochemistry (IHC) was used for determining the presence of NEO-101/102, NEO-201 and NEO-301 antigens on paraffin microarrays and tissues. These primary antibodies (NEO-101/NEO-102, NEO-201 and NEO-301) were biotinylated prior to use. Paraffin tissue microarrays and slides of tissues were reacted with the primary antibody after inactivating endogenous peroxidase and blocking the slides. Staining was detected with streptavidin-horseradish peroxidase conjugate and visualized with chromogen. Biotinylated human IgG1 was used as negative control and mouse anti-human cytokeratin monoclonal antibody was used as positive control. Antibody staining was measured as % and intensity of tumor epithelial cells and luminal secretion within the whole tissue section.

The cancer cells and normal cells tested were of the following tissue origins: Colon, Pancreas, Lung, Larynx, Prostate, Esophagus, Uterus, Stomach, Skin, Breast, and Ovary.

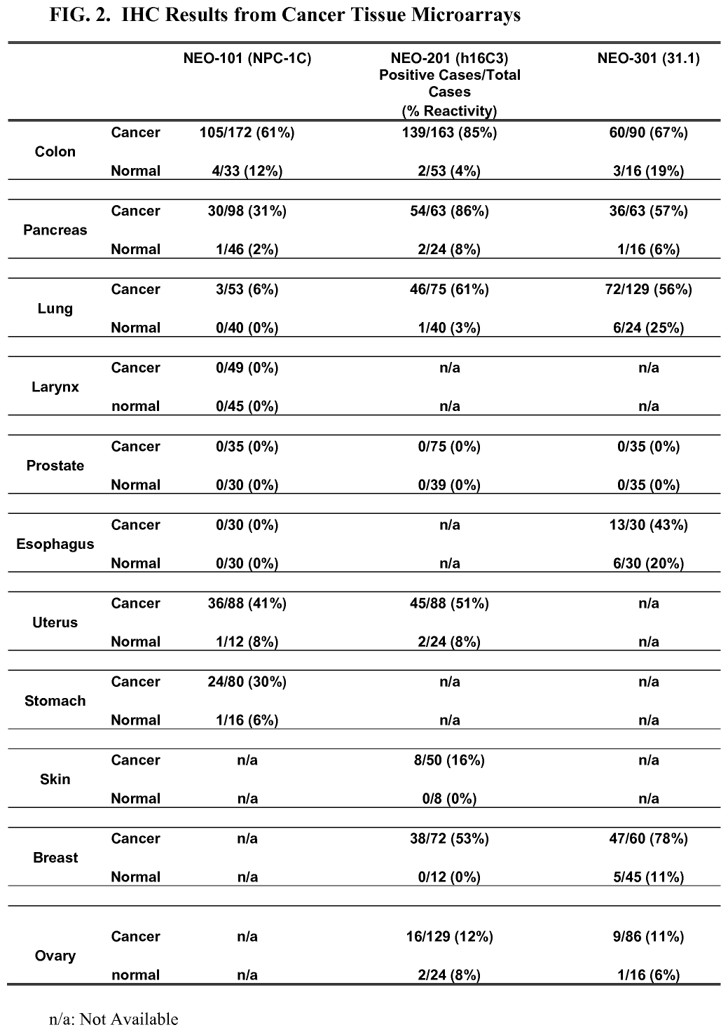

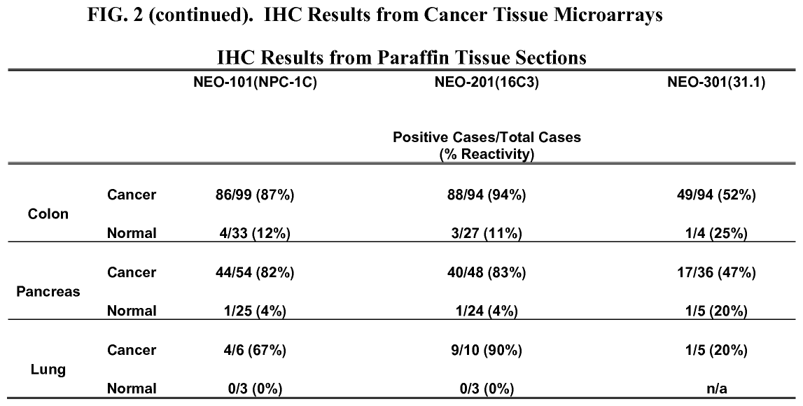

The results are summarized in tabular form in FIG. 2. For cancer tissue microarray samples, a high percentage of colon cancer samples were positive for all three antigens, with 61%, 85%, and 87% respectively, of the cancer samples being positive for NEO-101, NEO-201, and NEO-301. For pancreatic cancer a high percentage of cancer samples were positive for NEO-201 and NEO-301 at 86% and 57%, respectively, positive for the antigen; a lower but still appreciable fraction (31%) of pancreatic cancer cells were positive for NEO-101. For lung cancer a high percentage of cancer samples were positive for NEO-201 and NEO-301 at 61% and 56%, respectively. For esophagus cancer 43% of the cancer samples were positive for NEO-301, and for uterus cancer 41% of the cancer samples were positive for NEO-101 and 51% of cells were positive for NEO-201. For stomach cancer, 30% of samples were positive for NEO-101. For skin cancer, 16% of samples were positive for NEO-201. Finally, for breast cancer, 78% of samples were positive for NEO-301. Additionally, normal (non-cancerous) samples of each cell type were tested, with the percentages of positive samples as indicated which were generally low relative to the percentage of positive cancer cell samples in each instance.

For paraffin tissue sections, a high percentage of colon cancer samples were positive for all three antigens, with 87%, 94%, and 52% respectively, of the cancer samples being positive for NEO-101, NEO-201, and NEO-301. For pancreatic cancer a high percentage of cancer samples were positive for all three antigens, with 82%, 83%, and 47% respectively, of the cancer samples being positive for NEO-101, NEO-201, and NEO-301. For lung cancer a high percentage of samples were positive for NEO-101 and NEO-201 at 67% and 90%, respectively; relative to the other sample types relatively fewer lung cancer samples were tested which may account for some of the variation relative to the cancer tissue microarray results. Additionally, normal (non-cancerous) samples were tested as indicated, with the percentages of positive samples as indicated which were generally low relative to the percentage of positive cancer cell samples in each instance.

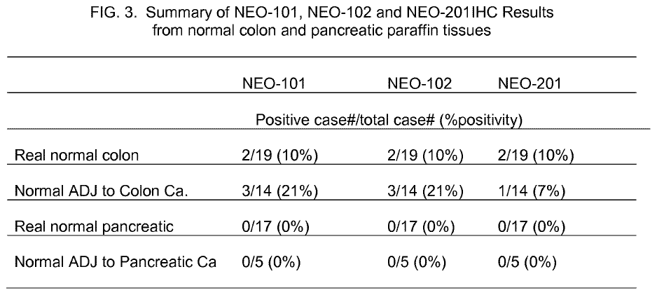

Finally, expression results for normal cell samples from paraffin tissue sections are summarized in FIG. 3 for both normal cell samples and normal samples adjacent to colon or pancreatic cancer.

### Example 3

This example reports the use of NEO-101, NEO-201, and NEO-301 to localize cancer cells in vivo.

Antibodies NEO-101, NEO-201, and NEO-301 are radiolabeled by conjugation to either ¹¹¹In or ⁸⁶Y using the acyclic chelate CHX-A″-DTPA. The conjugation molar ratios are empirically determined. Human tumor xenografts are introduced into SCID or nude mice, including the cell lines identified in FIG. 1 as being reactive with NEO-101, NEO-201, or NEO-301 in cell culture and optionally further cancer cell lines detected to bind those antibodies. Control xenografts non-reactive with these antibodies are also utilized. Following intravenous administration of radiolabeled antibodies in tumor-bearing mice, cell samples are harvested and analyzed. Labeled antibody biodistribution is determined with respect to the tumor and other tissues including liver and kidneys.

Additionally, xenograft bearing mice are subjected to imaging studies including planar gamma-imaging performed at multiple time points to assess the stability of labeled antibody binding to the tumor cells and measure the level of accumulation of radiolabeled antibody in other tissues. The study results establish that expression of the NEO-101, NEO-201, and NEO-301 antigens by cancer cells occurs in vivo, and that these antibodies are able to bind their respective antigens and thereby permit cancer detection in an in vivo context.

### Example 4

Different tumor models are assessed using radiolabeled antibodies according to the invention. These tumor models will comprise mice who contain human tumor cells originating from a human tumor cell line that expresses a target antigen bound by NEO-101, NEO-201 and/or NEO-301. Ideally these tumor models will correspond to different types of human cancers that express these antigens, e.g., colon, pancreatic, lung, prostate, melanoma, breast, melanoma, ovarian and mesothelioma. As the target antigens are known to be expressed by different cancers it is anticipated that human cancer cell lines tumor corresponding to these cancer types that express the subject target antigens may be identified by flow cytometry as described in Example 1.

In these experiments 4-6 week old male and female athymic mice (nu/nu) are subcutaneously injected in the flank or subscapular region with human cancer cell lines (about 2-6×106 cells) and tumors permitted to develop after injection. After the tumors have reached about 0.3-0.5 cm in diameter, these mice are injected with NEO-101, NEO-201 or NEO-301 antibody conjugates which are labeled with 111 In (prepared using standard labeling and purification methods). Approximately 5-7.5 micro Ci of the radiolabeled antibody is injected via the tail vein.

Mice (n=25) injected with the radiolabeled antibody are euthanized by CO2 inhalation at designated times, typically 24, 48, 72, 96 or 120 hours after injection. The blood, tumor and normal organs are harvested from the euthanized animals, wet weighed and the radioactivity measured in a gamma-scintillation counter. Radioactivity measurements (cpm's) are corrected for decay based on the day of injection and expressed as % ID/g of tissue. Tumor tissue ratios are calculated to determine the localization index for the RCs and blood tissue ratios are determined to assess the in vivo stability of the RCs.

In some instances a longer interval after injection, e.g., 14 days, is employed in order to better assess the residence time of the radiolabeled antibody in vivo. In such case additional mice are used in the studies.

The results of these experiments will confirm that different human tumors in vivo express the target antigens expressed by NEO-101, NEO-201 and NEO-301 in sufficient amounts to facilitate the use of these antibodies in labeled or unlabeled form to detect and treat different human cancers, e.g., colon, pancreatic, lung, prostate, melanoma, breast, melanoma, ovarian and mesothelioma. In addition, these experiments will confirm that the antibody is delivered to and localizes at the desired target sites (tumors) and not normal tissues.

In particular these experiments will corroborate that these antibodies specifically bind and accumulate in a time-dependent manner at tumor sites with little or no cross-reactivity with non-cancerous major organs and tissues such as the heart, spleen, kidney and liver. These results while obtained in a pre-clinical mouse model are anticipated to correlate to the binding properties of these antibodies in humans patients having a cancer which is characterized by expression of one or more of the target antigens bound by NEO-101, NEO-201 and NEO-301.

Based on these results the subject antibodies will be used to image tumors in vivo. These imaging methods provide for the early detection of cancers that express the antigens specifically bound by NEO-101, NEO-201 and NEO-301. In addition, these imaging methods are useful in cancer therapeutic regimens as these images may be used to stage the status of a cancer in a particular individual, and thereby design an appropriate therapeutic regimen using the subject antibodies alone or in association with other treatment regimens and therapeutic agents such as chemotherapy, radiation, immunoradiopharmaceuticals, other biologics and immune modulators and combinations thereof. The use of the subject antibodies as imaging agents may be used to establish disease prognosis based on the number and location of tumor cells that express the antigen bound by NEO-101, 201 or NEO-301 and further may be used to establish the efficacy of a particular treatment method based on its effect on the number and location of detected tumor cells and the level of tumor associated antigen expressed on these cells. Methods for using antibodies and antibody fragments to detect and image tumors in vivo are well established.

### Example 5

This example describes the determination of the involvement of extrinsic and intrinsic apoptosis pathways in cancer cell death after cell treatment with anti-CAA antibodies including NEO-201, and experiments conducted in cultured cells and in animal and human subjects to measure efficacy of anti-CAA antibodies (such as NEO-201) alone or in combination with another anti-cancer agent or therapy.

In brief, cancer cells (including tissue samples and cultures established from primary cell samples as well as cell lines) are first tested for binding of the cancer-associated anti-CAA antibodies (NEO-201, NEO-102, or NEO-301). Binding of the antibody to the cells is determined by immunohistochemistry or FACS. Cancer cells tested include breast, ovarian, uterine, and cervical cancer cells and tissues. From these results it is confirmed that a subset of patient samples express the cancer-associated antigen and thus the patients from which they are derived would be expected to respond to treatment with the respective anti-CAA antibodies (such as NEO-201) that specifically bound to that sample.

Cancer cells to which the antibody bound are then tested for antibody-mediated cell death. Cells are contacted with the anti-CAA antibody (NEO-201, NEO-102, or NEO-301) in vitro. Antibody sequences are as indicated in Table 1, above, including antibody NEO-201 containing the variable heavy and light chain sequences shown in SEQ ID NOs. 95 and 100. Cells are treated with the antibody over a range of concentrations. Cell death, ATP levels, and expression of Caspases 3 and 8 are detected using the methods described below. Multiple measurements are performed for each condition to allow statistical comparison of results. From these results the activation of caspase 3 and/or caspase 8 during cell killing is detected by, indicating the involvement of the intrinsic and extrinsic pathways, respectively, in the cell killing mechanism.

Apoptosis pathway involvement is confirmed by RNAi knockdown of the caspase 3 and caspase 8 genes (individually in combination). Cells are treated with the anti-CAA antibody (NEO-201, NEO-102, or NEO-301) after RNAi knockdown of caspase-3 and/or 8. Mirroring the Caspase Glo® results, decreased apoptosis following knockdown of caspase is predicted to confirms involvement of the apoptotic pathway (i.e., extrinsic or intrinsic) in anti-CAA antibody-mediated cell death. Specifically, decreased cell death in the caspase-3 knockdown is indicative of extrinsic pathway involvement, while decreased cell death in caspase knockdown is indicative of intrinsic pathway involvement.

Cultured cancer cells are additionally treated with the anti-CAA antibody (NEO-201, NEO-102, or NEO-301) in combination with chemotherapeutic and radiation therapies in order to detect combinations that enhance cell killing. Chemotherapeutic agents that activate extrinsic or intrinsic apoptosis pathways are predicted to increase cell killing based on the involvement of the extrinsic or intrinsic pathway in cell killing, respectively, as determined by Caspase Glo® and RNAi knockdown experiments described above.

Experiments are performed to test the cell killing effects of extrinsic pathway activating chemotherapeutic agents tested in combination with the anti-CAA antibodies (NEO-201, NEO-102, or NEO-301). These extrinsic pathway-targeting agents include agents that target PML-RARα, DR4 (TRAIL R1), and/or DR5 (TRAIL R2). Specific agents targeting the extrinsic pathway to be tested include TRAIL (human TRAIL polypeptide or an agonistic fragment thereof), Dr4 agonists, Dr5 agonists, and all trans retinoic acid (ATRA).

Experiments are also performed to test the cell killing effects of intrinsic pathway activating chemotherapeutic agents tested in combination with the anti-CAA antibodies (NEO-201, NEO-102, or NEO-301). These intrinsic pathway-targeting agents include agents that target Bcl-1, Bcl-XL, Bax, BCL-Xs and/or PML-RARα. These include agents that act directly on the mitochondrial inner membrane, agents that antagonize the antiapoptotic members of the Bcl-2 protein family, and agents that enhance the activity of the proapoptotic members of the Bcl-2 family of proteins such as Bax. Specific agents targeting the intrinsic pathway include arsenic trioxide, lonidamine (a derivative of indazole-3-carboxylic acid), antisense agents targeting Bcl-1 (such as Genasense, G3139 or oblimersen sodium), antisense agents targeting Bcl-XL, Bax, and BCL-Xs. Additional antisense agents target both Bcl-2 and Bcl-XL, or target clusterin (also known as testosterone-repressed prostate message 2). The intrinsic pathway-targeting agents also include small molecules. One group of small molecules recognizes the surface pocket of Bcl-2 or Bcl-XL, including Antimycin-A and derivatives thereof, HA14-1, and synthetic BH3 organic peptides.

Additionally, from involvement of the extrinsic or intrinsic apoptotic pathway in cell killing, it is predicted that chemotherapeutic agents that activate the common pathway or otherwise promote or sensitize cells to apoptosis would also enhance cancer cell killing. This prediction is confirmed by detecting cell killing by anti-CAA antibodies (NEO-201, NEO-102, or NEO-301) in combination with a pro-apoptotic agent.

Direct cell killing is also detected by detecting cell death caused by treatment with anti-CAA antibodies (NEO-201, NEO-102, or NEO-301) in the absence of caspases 3 and 8 (i.e., cell death in the caspase 3 and caspase 8 combined RNAi knockdown). From cell killing activity in the absence of both caspases it is predicted that the anti-CAA antibody potentiates direct cell killing. From these results, it is predicted that chemotherapeutic agents that enhance direct cell killing can result in increased cell death in combination with the anti-CAA antibody. Direct cell killing agents tested in combination with the anti-CAA antibodies (NEO-201, NEO-102, or NEO-301) including mixed lineage kinase domain like (MLKL) protein, rapamycin (RAP) or derivatives and/or analogs thereof, such as everolimus or RAD001; CCI-779, ABT578, SAR543, ascomycin (an ethyl analog of FK506), AP23573, AP23841, KU-0063794, INK-128, EX2044, EX3855, EX7518, or compounds that bind to the ATP-binding cleft of mTOR, such as AZD08055 and OSIO27.

Cell killing by ADCC in the presence of the anti-CAA antibodies (NEO-201, NEO-102, or NEO-301) is also tested. In brief, cells are contacted with the antibody in the presence of effector cells (such as PBMCs) and cancer cell survival is measured.

Based thereon, cancer cell killing is tested in animal models. Cancer cells (including breast, ovarian, cervical, or uterine) are grown in a tumor xenograft model. The animals are treated with the same anti-CAA antibodies confirmed to kill the cells in vitro, and cells are selected that were killed by each anti-CAA antibody (including NEO-201). The efficacy of the anti-CAA antibody in the tumor xenograft model is determined alone. Combinations with anti-cancer agents that exhibited enhanced cell killing in vitro are tested in vivo to demonstrate enhanced therapeutic efficacy in this model.

Finally, human subjects are treated with the anti-CAA antibody (such as NEO-201). Patients are selected for treatment based upon detection of binding to and/or killing of patient cancer cell samples. Alternatively, patients are selected for treatment because similar patient cell samples have previously been shown to bind to and/or be killed by the anti-CAA antibody. For example, a patient may be selected for treatment where cells of the same cancer type (e.g., breast, ovarian, cervical, and/or uterine) and optionally the same stage of cancer progression have been previously shown to bind to and/or be killed by the anti-CAA antibody. Patients may be treated with the anti-CAA antibody alone, or in combination with another anti-cancer agent or therapy. The other anti-cancer agent or therapy may be selected based upon observed increases in therapeutic efficacy observed in the aforementioned animal studies, and/or based upon enhanced cell killing efficacy observed for the combination in vitro.

Methods for the foregoing experiments are provided in further detail below. Protocols and steps not otherwise described may be carried out by methods known in the art based upon the foregoing description.

### Cell Death Assays

Cytotoxicity is measured by adding 20 μl of 3-(4,5-dimethylthiazol-2-yl)-2,5-diphenyltetrazolium bromide (MTT) (12 mm) to cells for 60 min. DMSO is added to solubilize the MTT formazan (reduced product) and measurements are taken at a wavelength of 570/620 nm. Apoptosis and necrosis are measured using a propidium iodide (PI) and allophycocyanin-conjugated Annexin V Apoptosis Detection kit (eBiosciences). Cells are collected using TrypLE™ (Invitrogen) and analyzed by flow cytometry on a BD FACSCalibur according to the manufacturer's protocol.

### ATP Assay

Intracellular ATP levels are measured by a CellTiter-Glo luminescent cell viability assay (Promega G7571) according to the manufacturer's instructions and read on a Victor3 1420 multilabel automated plate reader (PerkinElmer Life Sciences).

### Caspase Activity by Caspase-Glo® (Luciferase Assays)

A luciferase-based caspase assay is used to measure caspases-3/7 (Caspase-Glo® 3/7 Assay Systems, Promega) and caspase-8 (Caspase-Glo 8 Assay System, Promega) activity according to the manufacturer's protocol. Samples are read on a Victor3 1420 multilabel automated plate reader (PerkinElmer Life Sciences).

Real Time qPCR and RT-PCR

Total RNA is extracted using a GeneJET™ RNA Purification kit (Fermentas, Canada) and treated with DNase (Fermentas) to remove traces of genomic DNA. 1 μg of purified RNA is reverse transcribed using Moloney murine leukemia virus reverse transcriptase (Sigma) with random hexamer and oligo(dT) primers (Fermentas). cDNA is diluted accordingly, and 10-μl reactions are set up using Green-2-Go qPCR Mastermix to run qPCR. A CFX384 Touch™ Real-Time PCR Detection System (Bio-Rad) is used to obtain the raw threshold cycle (Ct) values. Results are analyzed using the 2-ΔCt formula normalizing target gene expression to housekeeping controls. For RT-PCR, 50-μl reactions are prepared using 25 ng of cDNA, and PCR is performed using Taq DNA polymerase (Fermentas). PCR products are resolved on a 2% agarose gel and stained for qualitative or quantitative analysis.

### Immunohistochemistry

Cells are fixed and stained with primary antibody specific for cleaved caspase 3 or cleaved caspase 8. Samples are then incubated with biotinylated secondary antibody, followed by avidin-linked horseradish peroxidase (HRP) and staining.

### ADCC Assay

PBMC effector cells are isolated from blood samples obtained by Ficoll-Hypaque density centrifugation. The target cells are incubated at 5×10⁶ cells/mL in complete growth media with 15 μL of 0.1% calcein-AM solution (Sigma-Aldrich) for 30 minutes at 37° C., in the presence of 5% CO2. The cells are washed twice with 15 mL of PBS-0.02% EDTA and resuspended in 1 mL complete growth medium. Fifty microliters (10,000 cells) of labeled target cells are plated into a 96-well plate in the presence or absence of antibodies at the selected concentration, and incubated with 50 μL of freshly isolated peripheral blood mononuclear cells (effector cells, at 100:1 E/T ratio) accordingly. After 2 hours of incubation, the plate is centrifuged at 300×g for 10 minutes, and 75 μL of supernatant is transferred into a new flat-bottomed 96-well plate. The fluorescence in the supernatant is measured at 485-nm excitation and 535-nm emission. Spontaneous release is determined from target cells in RPMI-1640 medium with 30% FBS without effector cells and maximum release is determined from target cells in RPMI-1640 medium with 30% FBS and 6% Triton X-100 without effector cells.

$$Percent cytotoxicity is calculated as [ ( counts in sample - spontaneous release ) / ( maximum counts - spontaneous release ) ] \times 100.$$

Each document cited herein including all patents, patent applications, non-patent literature, and any other publications, is hereby incorporated by reference in its entirety.

While the invention has been described by way of examples and preferred embodiments, it is understood that the words which have been used herein are words of description, rather than words of limitation. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

### Example 6

This example describes assays to determine cytotoxic effects of antibodies to cancer-associated antigens (including antibodies to NPC-1, 16C3, and 31-1 antigens such as NEO-102, NEO-102, NEO-103, NEO-201, NEO-301, and NEO-302) in a xenograft human cancer murine model, as a single agent and in combination with chemotherapy or radiation therapy.

Methods: The methods used are essentially as described in Buchsbaum et al., Clin Cancer Res. 2003 Sep. 1; 9(10 Pt 1):3731-41, which is hereby incorporated by reference in its entirety. In brief, the binding of the cancer-associated antigen antibody to a panel of human cancer cell lines is evaluated by indirect immunofluorescence and flow cytometry (also see Example 1). Cytotoxicity of the antibody alone and in the presence of an anti-cancer agent (gemcitabine or another agent) is measured in vitro. Antitumor efficacy is determined by treatment of nude mice bearing human cancer xenografts with the antibody alone or in combination with an anti-cancer agent, or in combination with radiation therapy. Tumor size and regression rates are determined. Optionally, expression of a cancer-associated antigen (such as the NPC-1, 16C3, or 31.1 antigen) may be measured to determine whether cancer-associated antigen expression is increased by an anti-cancer agent (gemcitabine or another agent) or radiation therapy, which without intent to be limited by theory would be predicted to increase antibody binding to cancer cells and/or increase susceptibility of cancer cells to depletion by the antibody. The results indicate that the antibodies alone or in combination with chemotherapy and/or radiation have antitumor efficacy in the cancer xenograft models, and synergistic benefits of the combination therapies are observed relative to treatment with the individual agents.

Each document cited herein, including all patent and non-patent literature, published or unpublished patent applications, abstracts, and any other document cited, is hereby incorporated by reference in its entirety.

Having fully described the invention, the following claims are now provided below.

SEQ ID NO: 1
MSVGRRKLALLWALALALACTRHTGHAQDGSSESSYKHHPALSPIARGPSGVPL
RGATVFPSLRTIPVVRASNPAHNGRVCSTWGSFHYKTFDGDVFRFPGLCNYVFSE
HCGAAYEDFNIQLRRSQESAAPTLSRVLMKVDGVVIQLTKGSVLVNGHPVLLPF
SQSGVLIQQSSSYTKVEARLGLVLMWNHDDSLLLELDTKYANKTCGLCGDFNG
MPVVSELLSHNTKLTPMEFGNLQKMDDPTEQCQDPVPEPPRNCSTGFGICEELLH
GQLFSGCVALVDVGSYLEACRQDLCFCEDTDLLSCVCHTLAEYSRQCTHAGGLP
QDWRGPDFCPQKCPNNMQYHECRSPCADTCSNQEHSRACEDHCVAGCFCPEGT
VLDDIGQTGCVPVSKCACVYNGAAYAPGATYSTDCTNCTCSGGRWSCQEVPCP
DTCSVLGGAHFSTFDGKQYTVHGDCSYVLTKPCDSSAFTVLAELRRCGLTDSET
CLKSVTLSLDGAQTVVVIKASGEVFLNQIYTQLPISAANVTIFRPSTFFIIAQTSLGL
QLNLQPVPTMQLFMQLAPKLRGQTCGLCGNFNSIQADDFRTLSGVVEATAAAFF
NTFKTQAACPNIRNSFEDPCSLSVENEKYAQHWCSQLTDADGPFGRCHAAVKPG
TYYSNCVFDTCNCERSEDCLCAALSSYVHACAAKGVQLGGWRDGVCTKPMTT
CPKSMTYHYHVSTCQPTCRSLSEGDITCSVGFIPVDGCICPKGTFLDDTGKCVQA
SNCPCYHRGSMIPNGESVHDSGAICTCTHGKLSCIGGQAPAPVCAAPMVFFDCRN
ATPGDTGAGCQKSCHTLDMTCYSPQCVPGCVCPDGLVADGEGGCITAEDCPCV
HNEASYRAGQTIRVGCNTCTCDSRMWRCTDDPCLATCAVYGDGHYLTFDGQSY
SFNGDCEYTLVQNHCGGKDSTQDSFRVVTENVPCGTTGTTCSKAIKIFLGGFELK
LSHGKVEVIGTDESQEVPYTIQQMGIYLVVDTDIGLVLLWDKKTSIFINLSPEFKG
RVCGLCGNFDDIAVNDFATRSRSVVGDVLEFGNSWKLSPSCPDALAPKDPCTAN
PFRKSWAQKQCSILHGPTFAACHAHVEPARYYEACVNDACACDSGGDCECFCT
AVAAYAQACHEVGLCVCLRTPSICPLFCDYYNPEGQCEWHYQPCGVPCLRTCR
NPRGDCLRDVRGLEGCYPKCPPEAPIFDEDKMQCVATCPTPPLPPRCHVHGKSY
RPGAVVPSDKNCQSCLCTERGVECTYKAEACVCTYNGQRFHPGDVIYHTTDGT
GGCISARCGANGTIERRVYPCSPTTPVPPTTFSFSTPPLVVSSTHTPSNGPSSAHTG
PPSSAWPTTAGTSPRTRLPTASASLPPVCGEKCLWSPWMDVSRPGRGTDSGDFD
TLENLRAHGYRVCESPRSVECRAEDAPGVPLRALGQRVQCSPDVGLTCRNREQA
SGLCYNYQIRVQCCTPLPCSTSSSPAQTTPPTTSKTTETRASGSSAPSSTPGTVSLS
TARTTPAPGTATSVKKTFSTPSPPPVPATSTSSMSTTAPGTSVVSSKPTPTEPSTSS
CLQELCTWTEWIDGSYPAPGINGGDFDTFQNLRDEGYTFCESPRSVQCRAESFPN
TPLADLGQDVICSHTEGLICLNKNQLPPICYNYEIRIQCCETVNVCRDITRLPKTVA
TTRPTPHPTGAQTQTTFTTHMPSASTEQPTATSRGGPTATSVTQGTHTTLVTRNC
HPRCTWTKWFDVDFPSPGPHGGDKETYNNIIRSGEKICRRPEEITRVQCRAKSHP
EVSIEHLGQVVQCSREEGLVCRNQDQQGPFKMCLNYEVRVLCCETPRGCHMTST
PGSTSSSPAQTTPSTTSKTTEIQASGSSAPSSTPGTVSLSTARTTPAPGTATSVKKTF
STPSPPPVPATSTSSMSTTAPGTSVVSSKPTPTEPSTSSCLQELCTWTEWIDGSYPA
PGINGGDFDTFQNLRDEGYTFCESPRSVQCRAESFPNTPLGRLGQDVICSHTEGLI
CLNKNQLPPICYNYEIRIQCCETVNVCRDITRPPKTVATTRPTPHPTGAQTQTTFT
THMPSASTEQPTATSRGGPTATSVTQGTHTTPVTRNCHPRCTWTTWFDVDFPSP
GPHGGDKETYNNIIRSGEKICRRPEEITRLQCRAKSHPEVSIEHLGQVVQCSREEG
LVCRNQDQQGPFKMCLNIEVRVLCCETPKGCPVTSTPVTAPSTPSGRAISPTQSTS
SWQKSRTTTLVTTSTTSTPQTSTTYAHTTSTTSAPTARTTSAPTTSTTSVPTTSTIS
GPKTTPSPVPTTSTTSAATTSTISAPTTSTTSVPGTTPSPVLTTSTTSAPTTRTTSASP
AGTTSGPGNTPSPVPTTSTISAPTTSITSAPTTSTTSAPTSSTTSGPGTTPSPVPTTSIT
SAPTTSTTSAPTTSTTSAPTTSTTSAPTTSTTSAPTTSTTSTPTSSTTSTPQTSTTSAS
TTSITSGPGTTPSPVPTTSTTSAPTTSTTSAATTSTISAPTTSTTSAPTTSTTSASTAS
KTSGLGTTPSPIPTTSTTSPPTTSTTSASTASKTSGPGTTPSPVPTTSTIFAPRTSTTS
ASTTSTTPGPGTTPSPVPTTSTASVSKTSTSHVSISKTTHSQPVTRDCHLRCTWTK
WFDVDFPSPGPHGGDKETYNNIIRSGEKICRRPEEITRLQCRAESHPEVSIEHLGQ
VVQCSREEGLVCRNQDQQGPFKMCLNYEVRVLCCETPKGCPVTSTPVTAPSTPS
GRATSPTQSTSSWQKSRTTTLVTTSTTSTPQTSTTSAPTTSTTSAPTTSTTSAPTTST
TSTPQTSISSAPTSSTTSAPTSSTISARTTSIISAPTTSTTSSPTTSTTSATTTSTTSAPT
SSTTSTPQTSKTSAATSSTTSSSGTTPSPVTTTSTASVSKTSTSHVSVSKTTHSQPVT
RDCHPRCTWTKWFDVDFPSPGPHGGDKETYNNIIRSGEKICRRPQEITRLQCRAK
SHPEVSIEHLGQVVQCSREEGLVCRNQDQQGPFKMCLNYEVRVLCCETPKGCPV
TSTSVTAPSPLVGEPPAQTQSTSSWQKSRTTTLVTSSITSTTQTSTTSAPTTSTTPAS
IPSTTSAPTTSTTSAPTTSTTSAPTTSTTSTPQTTTSSAPTSSTTSAPTTSTISAPTTSTI
SAPTTSTTSAPTASTTSAPTSTSSAPTTNTTSAPTTSTTSAPITSTISAPTTSTTSTPQT
STISSPTTSTTPTPQTSTTSSPTTSTTSAPTTSTTSAPTTSTTSTPQTSISSAPTSSTTSA
PTASTISAPTTSTTSFHTTSTTSPPTSSTSSTPQTSKTSAATSSTTSGSGTTPSPVPTT
STASVSKTSTSHVSVSKTTHSQPVTRDCHPRCTWTKWFDVDFPSPGPHGGDKET
YNNIIRSGEKICRRPEEITRLQCRAESHPEVSIEHLGQVVQCSREEGLVCRNQDQQ
GPFKMCLNYEVRVLCCETPKGCPVTSTPVTAPSTPSGRATSPTQSTSSWQKSRTT
TLVTTSTTSTPQTSTTSAPTTSTIPASTPSTTSAPTTSTTSAPTTSTTSAPTHRTTSGP
TTSTTLAPTTSTTSAPTTSTNSAPTTSTISASTTSTISAPTTSTISSPTSSTTSTPQTSK
TSAATSSTTSGSGTTPSPVPTTSTTSASTTSTTSAPTTSTTSGPGTTPSPVPSTSITSA
ATTSTTSAPTTRTTSAPTSSMTSGPGTTPSPVPTTSTTSAPTTSTTSGPGTTPSPVPT
TSTTSAPITSTTSGPGSTPSPVPTTSTTSAPTTSTTSASTASTTSGPTTSTTSASTTSTI
SPLTTSTTSAPITSMPSGPGTTPSPVPTTSTTSAPTTSTTSGPGTTPSPVPTTSTTSAP
TTSTTSASTASTTSGPGTTPSPVPTTSTTSAPTTSTTSASTASTTSGPGTSLSPVPTTS
TTSAPTTSTTSGPGTTPSPVPTTSTTSAPTTSTTSGPGTTPSPVPTTSTTPVSKTSTSH
LSVSKTTHSQPVTSDCHPLCAWTKWFDVDFPSPGPHGGDKETYNNIIRSGEKICR
RPEEITRLQCRAESHPEVNIEHLGQVVQCSREEGLVCRNQDQQGPFKMCLNYEV
RVLCCETPRGCPVTSVTPYGTSPTNALYPSLSTSMVSASVASTSVASSSVASSSVA
YSTQTCFCNVADRLYPAGSTIYRHRDLAGHCYYALCSQDCQVVRGVDSDCPSTT
LPPAPATSPSISTSEPVTELGCPNAVPPRKKGETWATPNCSEATCEGNNVISLSPRT
CPRVEKPTCANGYPAVKVADQDGCCHHYQCQCVCSGWGDPHYITFDGTYYTFL
DNCTYVLVQQIVPVYGHFRVLVDNYFCGAEDGLSCPRSIILEYHQDRVVLTRKP
VHGVMTNEIIFNNKVVSPGFRKNGIVVSRIGVKMYATIPELGVQVMFSGLIFSVE
VPFSKFANNTEGQCGTCTNDRKDECRTPRGTVVASCSEMSGLWNVSIPDQPACH
RPHPTPTTVGPTTVGSTTVGPTTVGSTTVGPTTPPAPCLPSPICHLILSKVFEPCHT
VIPPLLFYEGCVFDRCHMTDLDVVCSSLELYAALCASHDICIDWRGRTGHMCPFT
CPADKVYQPCGPSNPSYCYGNDSASLGALREAGPITEGCFCPEGMTLFSTSAQVC
VPTGCPRCLGPHGEPVKVGHTVGMDCQECTCEAATWTLTCRPKLCPLPPACPLP
GFVPVPAAPQAGQCCPQYSCACNTSRCPAPVGCPEGARAIPTYQEGACCPVQNC
SWTVCSINGTLYQPGAVVSSSLCETCRCELPGGPPSDAFVVSCETQICNTHCPVGF
EYQEQSGQCCGTCVQVACVTNTSKSPAHLFYPGETWSDAGNHCVTHQCEKHQD
GLVVVTTKKACPPLSCSLDEARMSKDGCCRFCPLPPPPYQNQSTCAVYHRSLIIQ
QQGCSSSEPVRLAYCRGNCGDSSSMYSLEGNTVEHRCQCCQELRTSLRNVTLHC
TDGSSRAFSYTEVEECGCMGRRCPAPGDTQHSEEAEPEPSQEAESGSWERGVPVS
PMH
SEQ ID NO: 2
ACCACCCATTCTCAACCTGTTACTCGTGATTGTCATCTGCGTTGCACCTGGACTA
AATGGTTCGACGTTGACTTCCCGTCCCCAGGTCCACACGGTGGTGACAAGGAAA
CCTATAACAACATCATTCGTTCCGGTGAGAAAATCTGCCGTCGTCCGGAGGAAAT
CACCCGTCTGCAGTGCCGTGCAGAGTCCCACCCGGAGGTATCTATCGAACATCTG
GGCCAGGTTGTGCAGTGCAGCCGTGAAGAAGGTCTGGTTTGCCGTAACCAAGAT
CAGCAGGGCCCGTTCAAAATGTGCCTGAACTATGAAGTCCGTGTCCTGTGCTGCG
AAACCCCAAAAGGCTGTCCAGTTACTTCTACCCCGGTTACCGCGCCGTCCACTCC
AAGCGGCCGCGCGACCAGCCCGACCCAGAGCACCTCCTCTTGGCAGAAATCCCG
CACCACTACCCTGGTTACTACCTCTACTACCTCCACTCCACAGACTTCCACCACC
TCCGCCCCGACTACCAGCACTACCAGCGCCCCGACCACTAGCACTACCTCCGCTC
CGACCACCTCCACCACTTCTACCCCGCAGACCTCTATCTCTTCTGCGCCGACCAG
CTCTACCACCAGCGCTCCGACTAGCTCCACGATTTCTGCTCGTACTACTTCTATCA
TTTCCGCCCCTACGACCTCTACCACTTCTAGCCCTACCACCTCTACCACGTCCGCG
ACCACCACCTCCACTACCTCTGCACCAACTTCCTCTACTACGAGCACGCCGCAGA
CTTCTAAAACCTCTGCGGCAACCTCTTCTACCACCAGCAGCTCTGGCACCACTCC
GAGCCCGGTGACCACCACTAGCACCGCTTCTGTGTCCAAGACCAGCACCTCTCAC
GTGTCTGTTTCTAAAACGACCCACTCCCAGCCGGTTACCCGC
SEQ ID NO: 3
TTHSQPVTRDCHLRCTWTKWFDVDFPSPGPHGGDKETYNNIIRSGEKICRRPEEIT
RLQCRAESHPEVSIEHLGQVVQCSREEGLVCRNQDQQGPFKMCLNYEVRVLCCE
TPKGCPVTSTPVTAPSTPSGRATSPTQSTSSWQKSRTTTLVTTSTTSTPQTSTTSAP
TTSTTSAPTTSTTSAPTTSTTSTPQTSISSAPTSSTTSAPTSSTISARTTSIISAPTTSTT
SSPTTSTTSATTTSTTSAPTSSTTSTPQTSKTSAATSSTTSSSGTTPSPVTTTSTASVS
KTTSHVSVSKTTHSQPVTR
SEQ ID NO: 4
ACTACTCATTCTCAACCTGTAACTCGTGATTGTCATCTGCGCTGTACTTGGAC
TAAATGGTTTGACGTGGACTTCCCGTCCCCTGGCCCGCACGGTGGTGATAAA
GAAACCTACAATAACATCATTCGCTCTGGTGAGAAAATCTGCCGTCGTCCGG
AAGAAATCACTCGTCTGCAATGTCGTGCCGAATCCCACCCGGAGGTGAGCAT
CGAACACCTGGGTCAGGTTGTTCAGTGTTCTCGTGAGGAAGGTCTGGTATGCC
GTAACCAAGATCAGCAAGGCCCATTCAAAATGTGCCTGAACTACGAAGTTCG
TGTTCTGTGTTGCGAGACTCCGAAAGGTTGCCCGGTTACGAGCACGCCTGTCA
CCGCACCGAGCACGCCG
SEQ ID NO: 5
TTHSQPVTRDCHLRCTWTKWFDVDFPSPGPHGGDKETYNNIIRSGEKICRRPEEIT
RLQCRAESHPEVSIEHLGQVVQCSREEGLVCRNQDQQGPFKMCLNYEVRVLCCE
TPKGCPVTSTPVTAPSTP
SEQ ID NO: 6
LVTTSTTSTPQTSTTSAPTTSTTSAPTTSTTSAPTTSTTSTPQTS
SEQ ID NO: 7
SSPTTSTTPTPQTSTTSSPTTSTTSAPTTSTTSAPTTSTTSTPQTS
SEQ ID NO: 8
ITSAPTTSTTSAPTTSTTSAPTTSTTSAPTTSTTSAPTTSTTSTPTSSTTSTP
QTSTTS
SEQ ID NO: 9
ASIPSTTSAPTTSTTSAPTTSTTSAPTTSTTSTPQTTTSSAPTSSTTSAPTTST
SEQ ID NO: 10
MTSGPGTTPSPVPTTSTTSAPTTSTTSGPGTTPSPVPTTSTTSAP
SEQ ID NO: 11
ITSMPSGPGTTPSPVPTTSTTSAPTTSTTSGPGTTPSPVPTTSTTSAPTTSTTS
SEQ ID NO: 12
LSPVPTTSTTSAPTTSTTSGPGTTPSPVPTTSTTSAPTTSTTSGPGTTPSPVP
TTSTTP
SEQ ID NO: 13
ATMSVGRRKLALLWALALALACTTTHSQPVTRDCHLRCTWTKWFDVDFPSPGP
HGGDKETYNNIIRSGEKICRRPEEITRLQCRA
SEQ ID NO: 14
ATMSVGRRKLALLWALALALACTTTHSQPVTRDCHLRCTWTKWFDVDFPSPGP
HGGDKETYNNIIRSGEKICRRPEEITRLQCRAESHPEVSIEHLGQVVQCSREEGLV
CRNQDQQGPFKMCLNYEVRVLCCETPK
SEQ ID NO: 15
ATMSVGRRKLALLWALALALACTTTHSQPVTRDCHLRCTWTKWFDVDFPSPGP
HGGDKETYNNIIRSGEKICRRPEEITRLQCRAESHPEVSIEHLGQVVQCSREEGLV
CRNQDQQGPFKMCLNYEVRVLCCETPKGCPVTSTPVTAPSTP
SEQ ID NO: 16
ATMSVGRRKLALLWALALALACTTTHSQPVTRDCHLRCTWTKWFDVDFPSPGP
HGGDKETYNNIIRSGEKICRRPEEITRLQCRAESHPEVSIEHLGQVVQCSREEGLV
CRNQDQQGPFKMCLNYEVRVLCCETPKGCPVTSTPVTAPSTPSGRATSPTQSTSS
WQKSRTTTLVTTSTTSTPQTSTT
SEQ ID NO: 17
ATMSVGRRKLALLWALALALACTTTHSQPVTRDCHLRCTWTKWFDVDFPSPGP
HGGDKETYNNIIRSGEKICRRPEEITRLQCRAESHPEVSIEHLGQVVQCSREEGLV
CRNQDQQGPFKMCLNYEVRVLCCETPKGCPVTSTPVTAPSTPSGRATSPTQSTSS
WQKSRTTTLVTTSTTSTPQTSTTSAPTTSTTSAPTTSTTSAPTTSTTSTPQTSISSAP
TSSTTSAPTSSTISARTTSIISAPTTSTTSSPTTSTTSATTTSTTSAPTSSTTSTPQTSKT
SAATSS
SEQ ID NO: 18
ATMSVGRRKLALLWALALALACTTTHSQPVTRDCHLRCTWTKWFDVDFPSPGP
HGGDKETYNNIIRSGEKICRRPEEITRLQCRAESHPEVSIEHLGQVVQCSREEGLV
CRNQDQQGPFKMCLNYEVRVLCCETPKGCPVTSTPVTAPSTPSGRATSPTQSTSS
WQKSRTTTLVTTSTTSTPQTSTTSAPTTSTTSAPTTSTTSAPTTSTTSTPQTSISSAP
TSSTTSAPTSSTISARTTSIISAPTTSTTSSPTTSTTSATTTSTTSAPTSSTTSTPQTSKT
SAATSSTTSSSGTTPSPVTTTSTASVSKTSTSHVSVSKTTHSQPVTRCTHHHHHH
SEQ ID NO: 19
MESPSAPPHRWCIPWQRLLLTASLLTFWNPPTTAKLTIESTPFNVAEGKEVLLLV
HNLPQHLFGYSWYKGERVDGNRQIIGYVIGTQQATPGPAYSGREIIYPNASLLIQN
IIQNDTGFYTLHVIKSDLVNEEATGQFRVYPELPKPSISSNNSKPVEDKDAVAFTC
EPETQDATYLWWVNNQSLPVSPRLQLSNGNRTLTLFNVTRNDTASYKCETQNPV
SARRSDSVILNVLYGPDAPTISPLNTSYRSGENLNLSCHAASNPPAQYSWFVNGT
FQQSTQELFIPNITVNNSGSYTCQAHNSDTGLNRTTVTTITVYAEPPKPFITSNNSN
PVEDEDAVALTCEPEIQNTTYLWWVNNQSLPVSPRLQLSNDNRTLTLLSVTRND
VGPYECGIQNISPPDSSYLSGANLNLSCHSASNPSPQYSWRINGIPQQHTQVLFIAK
ITPNNNGTYACFVSNLATGRNNSIVKSITVSASGTSPGLSAGATVGIMIGVLVGVA
LI
SEQ ID NO: 20
GAAGAGACTCAGGGCAGAGGGAGGAAGGACAGCAGACCAGACAGTCACAG
CAGCCTTGACAAAACGTTCCTGGAACTCAAGCTCTTCTCCACAGAGGAGGAC
AGAGCAGACAGCAGAGACCATGGAGTCTCCCTCGGCCCCTCCCCACAGATGG
TGCATCCCCTGGCAGAGGCTCCTGCTCACAGCCTCACTTCTAACCTTCTGGAA
CCCGCCCACCACTGCCAAGCTCACTATTGAATCCACGCCGTTCAATGTCGCAG
AGGGGAAGGAGGTGCTTCTACTTGTCCACAATCTGCCCCAGCATCTTTTTGGC
TACAGCTGGTACAAAGGTGAAAGAGTGGATGGCAACCGTCAAATTATAGGAT
ATGTAATAGGAACTCAACAAGCTACCCCAGGGCCCGCATACAGTGGTCGAGA
GATAATATACCCCAATGCATCCCTGCTGATCCAGAACATCATCCAGAATGAC
ACAGGATTCTACACCCTACACGTCATAAAGTCAGATCTTGTGAATGAAGAAG
CAACTGGCCAGTTCCGGGTATACCCGGAGCTGCCCAAGCCCTCCATCTCCAG
CAACAACTCCAAACCCGTGGAGGACAAGGATGCTGTGGCCTTCACCTGTGAA
CCTGAGACTCAGGACGCAACCTACCTGTGGTGGGTAAACAATCAGAGCCTCC
CGGTCAGTCCCAGGCTGCAGCTGTCCAATGGCAACAGGACCCTCACTCTATT
CAATGTCACAAGAAATGACACAGCAAGCTACAAATGTGAAACCCAGAACCC
AGTGAGTGCCAGGCGCAGTGATTCAGTCATCCTGAATGTCCTCTATGGCCCG
GATGCCCCCACCATTTCCCCTCTAAACACATCTTACAGATCAGGGGAAAATCT
GAACCTCTCCTGCCACGCAGCCTCTAACCCACCTGCACAGTACTCTTGGTTTG
TCAATGGGACTTTCCAGCAATCCACCCAAGAGCTCTTTATCCCCAACATCACT
GTGAATAATAGTGGATCCTATACGTGCCAAGCCCATAACTCAGACACTGGCC
TCAATAGGACCACAGTCACGACGATCACAGTCTATGCAGAGCCACCCAAACC
CTTCATCACCAGCAACAACTCCAACCCCGTGGAGGATGAGGATGCTGTAGCC
TTAACCTGTGAACCTGAGATTCAGAACACAACCTACCTGTGGTGGGTAAATA
ATCAGAGCCTCCCGGTCAGTCCCAGGCTGCAGCTGTCCAATGACAACAGGAC
CCTCACTCTACTCAGTGTCACAAGGAATGATGTAGGACCCTATGAGTGTGGA
ATCCAGAACAAATTAAGTGTTGACCACAGCGACCCAGTCATCCTGAATGTCC
TCTATGGCCCAGACGACCCCACCATTTCCCCCTCATACACCTATTACCGTCCA
GGGGTGAACCTCAGCCTCTCCTGCCATGCAGCCTCTAACCCACCTGCACAGT
ATTCTTGGCTGATTGATGGGAACATCCAGCAACACACACAAGAGCTCTTTATC
TCCAACATCACTGAGAAGAACAGCGGACTCTATACCTGCCAGGCCAATAACT
CAGCCAGTGGCCACAGCAGGACTACAGTCAAGACAATCACAGTCTCTGCGGA
GCTGCCCAAGCCCTCCATCTCCAGCAACAACTCCAAACCCGTGGAGGACAAG
GATGCTGTGGCCTTCACCTGTGAACCTGAGGCTCAGAACACAACCTACCTGT
GGTGGGTAAATGGTCAGAGCCTCCCAGTCAGTCCCAGGCTGCAGCTGTCCAA
TGGCAACAGGACCCTCACTCTATTCAATGTCACAAGAAATGACGCAAGAGCC
TATGTATGTGGAATCCAGAACTCAGTGAGTGCAAACCGCAGTGACCCAGTCA
CCCTGGATGTCCTCTATGGGCCGGACACCCCCATCATTTCCCCCCCAGACTCG
TCTTACCTTTCGGGAGCGAACCTCAACCTCTCCTGCCACTCGGCCTCTAACCC
ATCCCCGCAGTATTCTTGGCGTATCAATGGGATACCGCAGCAACACACACAA
GTTCTCTTTATCGCCAAAATCACGCCAAATAATAACGGGACCTATGCCTGTTT
TGTCTCTAACTTGGCTACTGGCCGCAATAATTCCATAGTCAAGAGCATCACAG
TCTCTGCATCTGGAACTTCTCCTGGTCTCTCAGCTGGGGCCACTGTCGGCATC
ATGATTGGAGTGCTGGTTGGGGTTGCTCTGATATAGCAGCCCTGGTGTAGTTT
CTTCATTTCAGGAAGACTGACAGTTGTTTTGCTTCTTCCTTAAAGCATTTGCA
ACAGCTACAGTCTAAAATTGCTTCTTTACCAAGGATATTTACAGAAAAGACTC
TGACCAGAGATCGAGACCATCCTAGCCAACATCGTGAAACCCCATCTCTACT
AAAAATACAAAAATGAGCTGGGCTTGGTGGCGCGCACCTGTAGTCCCAGTTA
CTCGGGAGGCTGAGGCAGGAGAATCGCTTGAACCCGGGAGGTGGAGATTGC
AGTGAGCCCAGATCGCACCACTGCACTCCAGTCTGGCAACAGAGCAAGACTC
CATCTCAAAAAGAAAAGAAAAGAAGACTCTGACCTGTACTCTTGAATACAAG
TTTCTGATACCACTGCACTGTCTGAGAATTTCCAAAACTTTAATGAACTAACT
GACAGCTTCATGAAACTGTCCACCAAGATCAAGCAGAGAAAATAATTAATTT
CATGGGACTAAATGAACTAATGAGGATAATATTTTCATAATTTTTTATTTGAA
ATTTTGCTGATTCTTTAAATGTCTTGTTTCCCAGATTTCAGGAAACTTTTTTTC
TTTTAAGCTATCCACAGCTTACAGCAATTTGATAAAATATACTTTTGTGAACA
AAAATTGAGACATTTACATTTTCTCCCTATGTGGTCGCTCCAGACTTGGGAAA
CTATTCATGAATATTTATATTGTATGGTAATATAGTTATTGCACAAGTTCAAT
AAAAATCTGCTCTTTGTATGACAGAATACATTTGAAAACATTGGTTATATTAC
CAAGACTTTGACTAGAATGTCGTATTTGAGGATATAAACCCATAGGTAATAA
ACCCACAGGTACTACAAACAAAGTCTGAAGTCAGCCTTGGTTTGGCTTCCTA
GTGTCAATTAAACTTCTAAAAGTTTAATCTGAGATTCCTTATAAAAACTTCCA
GCAAAGCAACTTTAAAAAAGTCTGTGTGGGCCGGGCGCGGTGGCTCACGCCT
GTAATCCCAGCACTTTGATCCGCCGAGGCGGGCGGATCACGAGGTCAGGAGA
TCCAGACCATCCTGGCTAACACAGTGAAACCCCGTCTCTACTAAAAATACAA
AAAAAGTTAGCCGGGCGTGGTGGTGGGGGCCTGTAGTCCCAGCTACTCAGGA
GGCTGAGGCAGGAGAACGGCATGAACCCGGGAGGCAGGGCTTGCAGTGAGC
CAAGATCATGCCGCTGCACTCCAGCCTGGGAGACAAAGTGAGACTCCGTCAA
AAAAAAAAAAAAGTCTATGTGGTCAGTCACTACTCTTGCTGCAGTTATGAAA
AGAATGAGGCCAAGTCTGATGAAAATAAACTTATTTTGAAAACA
SEQ ID NO: 21
MGPPSAPPCRLHVPWKEVLLTASLLTFWNPPTTAKLTIESTPFNVAEGKEVLLLA
HNLPQNRIGYSWYKGERVDGNSLIVGYVIGTQQATPGPAYSGRETIYPNASLLIQ
NVTQNDTGFYTLQVIKSDLVNEEATGQFHVYPELPKPSISSNNSNPVEDKDAVAF
TCEPEVQNTTYLWWVNGQSLPVSPRLQLSNGNMTLTLLSVKRNDAGSYECEIQN
PASANRSDPVTLNVLYGPDGPTISPSKANYRPGENLNLSCHAASNPPAQYSWFIN
GTFQQSTQELFIPNITVNNSGSYMCQAHNSATGLNRTTVTMITVSGSAPVLSAVA
TVGITIGVLARVALI
SEQ ID NO: 22
GACCCTGGGAAATGCTTCTATCCCTGAGAGGAGGCTCAGCACAGAAGGAGGA
AGGACAGCAGGGCCAACAGTCACAGCAGCCCTGACCAGAGCATTCCTGGAG
CTCAAGCTCCTCTACAAAGAGGTGGACAGAGAAGACAGCAGAGACCATGGG
ACCCCCCTCAGCCCCTCCCTGCAGATTGCATGTCCCCTGGAAGGAGGTCCTGC
TCACAGCCTCACTTCTAACCTTCTGGAACCCACCCACCACTGCCAAGCTCACT
ATTGAATCCACGCCGTTCAATGTCGCAGAGGGGAAGGAGGTTCTTCTACTCG
CCCACAACCTGCCCCAGAATCGTATTGGTTACAGCTGGTACAAAGGCGAAAG
AGTGGATGGCAACAGTCTAATTGTAGGATATGTAATAGGAACTCAACAAGCT
ACCCCAGGGCCCGCATACAGTGGTCGAGAGACAATATACCCCAATGCATCCC
TGCTGATCCAGAACGTCACCCAGAATGACACAGGATTCTATACCCTACAAGT
CATAAAGTCAGATCTTGTGAATGAAGAAGCAACCGGACAGTTCCATGTATAC
CCGGAGCTGCCCAAGCCCTCCATCTCCAGCAACAACTCCAACCCCGTGGAGG
ACAAGGATGCTGTGGCCTTCACCTGTGAACCTGAGGTTCAGAACACAACCTA
CCTGTGGTGGGTAAATGGTCAGAGCCTCCCGGTCAGTCCCAGGCTGCAGCTG
TCCAATGGCAACATGACCCTCACTCTACTCAGCGTCAAAAGGAACGATGCAG
GATCCTATGAATGTGAAATACAGAACCCAGCGAGTGCCAACCGCAGTGACCC
AGTCACCCTGAATGTCCTCTATGGCCCAGATGGCCCCACCATTTCCCCCTCAA
AGGCCAATTACCGTCCAGGGGAAAATCTGAACCTCTCCTGCCACGCAGCCTC
TAACCCACCTGCACAGTACTCTTGGTTTATCAATGGGACGTTCCAGCAATCCA
CACAAGAGCTCTTTATCCCCAACATCACTGTGAATAATAGCGGATCCTATATG
TGCCAAGCCCATAACTCAGCCACTGGCCTCAATAGGACCACAGTCACGATGA
TCACAGTCTCTGGAAGTGCTCCTGTCCTCTCAGCTGTGGCCACCGTCGGCATC
ACGATTGGAGTGCTGGCCAGGGTGGCTCTGATATAGCAGCCCTGGTGTATTTT
CGATATTTCAGGAAGACTGGCAGATTGGACCAGACCCTGAATTCTTCTAGCTC
CTCCAATCCCATTTTATCCCATGGAACCACTAAAAACAAGGTCTGCTCTGCTC
CTGAAGCCCTATATGCTGGAGATGGACAACTCAATGAAAATTTAAAGGGAAA
ACCCTCAGGCCTGAGGTGTGTGCCACTCAGAGACTTCACCTAACTAGAGACA
GGCAAACTGCAAACCATGGTGAGAAATTGACGACTTCACACTATGGACAGCT
TTTCCCAAGATGTCAAAACAAGACTCCTCATCATGATAAGGCTCTTACCCCCT
TTTAATTTGTCCTTGCTTATGCCTGCCTCTTTCGCTTGGCAGGATGATGCTGTC
ATTAGTATTTCACAAGAAGTAGCTTCAGAGGGTAACTTAACAGAGTATCAGA
TCTATCTTGTCAATCCCAACGTTTTACATAAAATAAGAGATCCTTTAGTGCAC
CCAGTGACTGACATTAGCAGCATCTTTAACACAGCCGTGTGTTCAAATGTACA
GTGGTCCTTTTCAGAGTTGGACTTCTAGACTCACCTGTTCTCACTCCCTGTTTT
AATTCAACCCAGCCATGCAATGCCAAATAATAGAATTGCTCCCTACCAGCTG
AACAGGGAGGAGTCTGTGCAGTTTCTGACACTTGTTGTTGAACATGGCTAAA
TACAATGGGTATCGCTGAGACTAAGTTGTAGAAATTAACAAATGTGCTGCTT
GGTTAAAATGGCTACACTCATCTGACTCATTCTTTATTCTATTTTAGTTGGTTT
GTATCTTGCCTAAGGTGCGTAGTCCAACTCTTGGTATTACCCTCCTAATAGTC
ATACTAGTAGTCATACTCCCTGGTGTAGTGTATTCTCTAAAAGCTTTAAATGT
CTGCATGCAGCCAGCCATCAAATAGTGAATGGTCTCTCTTTGGCTGGAATTAC
AAAACTCAGAGAAATGTGTCATCAGGAGAACATCATAACCCATGAAGGATA
AAAGCCCCAAATGGTGGTAACTGATAATAGCACTAATGCTTTAAGATTTGGT
CACACTCTCACCTAGGTGAGCGCATTGAGCCAGTGGTGCTAAATGCTACATA
CTCCAACTGAAATGTTAAGGAAGAAGATAGATCCAATTAAAAAAAATTAAAA
CCAATTTAAAAAAAAAAAGAACACAGGAGATTCCAGTCTACTTGAGTTAGCA
TAATACAGAAGTCCCCTCTACTTTAACTTTTACAAAAAAGTAACCTGAACTAA
TCTGATGTTAACCAATGTATTTATTTCTGTGGTTCTGTTTCCTTGTTCCAATTT
GACAAAACCCACTGTTCTTGTATTGTATTGCCCAGGGGGAGCTATCACTGTAC
TTGTAGAGTGGTGCTGCTTTAATTCATAAATCACAAATAAAAGCCAATTAGCT
CTATAACT
SEQ ID NO: 23
LQLSNGNMTLTLLSVKRNDAGSYECEIQNPASANRSDPVTLNVLYGPDGPTISPS
KANYRPGENLNLSCHAASNPPAQYSWFINGTFQQSTQELFIPNITVNNSGSYMCQ
AHNSATGLNRTTVTMITVS
SEQ ID NO: 24
LQLSNGNRTLTLFNVTRNDTASYKCETQNPVSARRSDSVILNVLYGPDAPTISPL
NTSYRSGENLNLSCHAASNPPAQYSWFVNGTFQQSTQELFIPNITVNNSGSYTCQ
AHNSDTGLNRTTVTTITVYA
SEQ ID NO: 25
LQLSNGNRTLTLLSVTRNDVGPYECEIQNPASANFSDPVTLNVLYGPDAPTISPSD
TYYHAGVNLNLSCHAASNPPSQYSWSVNGTFQQYTQKLFIPNITTKNSGSYACH
TTNSATGRNRTTVRMITVSD
SEQ ID NO: 26
LQLSNGNRTLTLLSVTRNDTGPYECEIQNPVSANRSDPVTLNVTYGPDTPTISPSD
TYYRPGANLSLSCYAASNPPAQYSWLINGTFQQSTQELFIPNITVNNSGSYTCHA
NNSVTGCNRTTVKTIIVTE
SEQ ID NO: 27
TTSTTSAP
SEQ ID NO: 28
GSTPSPVP
SEQ ID NO: 29
TASTTSGP
SEQ ID NO: 30
GTTPSPVPTTSTTSAP
SEQ ID NO: 31
GTTPSA VPTTSTTSVP
SEQ ID NO: 32
GTTPSPVPTTSITSVP
SEQ ID NO: 33
TTSTTSAPTTS
SEQ ID NO: 34
TTHSQPVTRDCHLRCTWTKWFDVDFPSPGPHGGDKETYNNIIRSGEKICRRPEEIT
RLQCRAESHPEVSIEHLGQVVQCSREEGLVCRNQDQQGPFKMCLNYEVRVLCCE
TPKGCPVTSTPVTAPSTPSGRATSPTQSTSSWQKSRTTTLVTTSTTSTPQTSTTSAP
TTSTTSAPTTSTTSAPTTSTTSTPQTSISSAPTSSTTSAPTSSTISARTTSIISAPTTSTT
SSPTTSTTSATTTSTTSAPTSSTTSTPQTSKTSAATSSTTSSSGTTPSPVTTTSTASVS
KTSTSHVSVSKTTHSQPVTR
SEQ ID NO: 35
GTHTTPVTRNCHPRCTWTKWFDVDFPSPGPHGGDKETYNNIIRSGEKICRRPEEIT
RLQCRAKSHPEVSIEHLGQVVQCSREEGLVCRNQDQQGPFKMCLNYEVRVLCCE
TPRGCPVTSTPVTAPSTPSGRAISPTQSTSSWQKSRTTTLVTTSTTSTPQTSTTYAH
TTSTTSAPTARTTSAPTTRTTSASPASTTSGPGNTPSPVPTTSTISAPTTSITSAPTTS
TTSAPTSSTTSAPTTSTTSAPTSSTTSSPQTSTTSAPTTSTTSGPGTTPSPVPTT
SEQ ID NO: 36
TTSTTSAPTTSTTSAP
SEQ ID NO: 37
GCPVTSTPVTAPSTP
SEQ ID NO: 38
MGHLSAPLHRVRVPWQGLLLTASLLTFWNPPTTAQLTTESMPFNVAEGKEVLLL
VHNLPQQLFGYSWYKGERVDGNRQIVGYAIGTQQATPGPANSGRETIYPNASLLI
QNVTQNDTGFYTLQVIKSDLVNEEATGQFHVYPELPKPSISSNNSNPVEDKDAVA
FTCEPETQDTTYLWWINNQSLPVSPRLQLSNGNRTLTLLSVTRNDTGPYECEIQN
PVSANRSDPVTLNVTYGPDTPTISPSDTYYRPGANLSLSCYAASNPPAQYSWLIN
GTFQQSTQELFIPNITVNNSGSYTCHANNSVTGCNRTTVKTIIVTELSPVVAKPQIK
ASKTTVTGDKDSVNLTCSTNDTGISIRWFFKNQSLPSSERMKLSQNALPQENGLS
PGAIAGIVIGVVALVALIAVALACFLHFGKTGSSGPLQ
SEQ ID NO: 39
MGPISAPSCRWRIPWQGLLLTASLFTFWNPPTTAQLTIEAVPSNAAEGKEVLLLV
HNLPQDPRGYNWYKGETVDANRRIIGYVISNQQITPGPAYSNRETIYPNASLLMR
NVTRNDTGSYTLQVIKLNLMSEEVTGQFSVHPETPKPSISSNNSNPVEDKDAVAF
TCEPETQNTTYLWWVNGQSLPVSPRLQLSNGNRTLTLLSVTRNDVGPYECEIQNP
ASANFSDPVTLNVLYGPDAPTISPSDTYYHAGVNLNLSCHAASNPPSQYSWSVN
GTFQQYTQKLFIPNITTKNSGSYACHTTNSATGRNRTTVRMITVSDALVQGSSPG
LSARATVSIMIGVLARVALI
SEQ ID NO: 40
GPDAPTIXXXXXXXXXXXXXXXXXXXXXXXXXXXXXXXXXXXXXXXXXXXX
XXXXXXXXXXXXXXXXGSYTCQAHNSDTGLNRTTVTTITVY
SEQ ID NO: 41
GPDAPTI
SEQ ID NO: 42
GSYTCQAHNSDTGLNRTTVTTITVY
SEQ ID NO: 43
GPDGPTIXXXXXXXXXXXXXXXXXXXXXXXXXXXXXXXXXXXXXXXXXXXX
XXXXXXXXXXXXXXXXGSYMCQAHNSATGLNRTTVTMITVS
SEQ ID NO: 44
GSYMCQAHNSATGLNRTTVTMITVS
SEQ ID NO: 45
MVGKMWPVLWTLCAVRVTVDAISVETPQDVLRASQGKSVTLPCTYHTSTSSRE
GLIQWDKLLLTHTERVVIWPFSNKNYIHGELYKNRVSISNNAEQSDASITIDQLTM
ADNGTYECSVSLMSDLEGNTKSRVRLLVLVPPSKPECGIEGETIIGNNIQLTCQSK
EGSPTPQYSWKRYNILNQEQPLAQPASGQPVSLKNISTDTSGYYICTSSNEEGTQF
CNITVAVRSPSMNVALYVGIAVGVVAALIIIGIIIYCCCCRGKDDNTEDKEDARPN
REAYEEPPEQLRELSREREEEDDYRQEEQRSTGRESPDHLDQ
SEQ ID NO: 46
ASQGKSVTLPCTYHTSTSSREGLIQWDKLLLTHTERVVIWPFSNKNYIHGELYKN
R
SEQ ID NO: 47
VRLLVLVPPSKPECGIEGETIIGNNIQLTCQSKEGSPTPQYSWKRYNILNQEQPLAQ
PASGQPVSLK
SEQ ID NO: 48
DDNTEDKEDARPNREAYEEPPEQLRELSREREEEDDYRQEEQRSTGRESPDHLD
Q
SEQ ID NO: 49
FSNKNYIHGEL YKNR
SEQ ID NO: 50
SPTPQYSWKRYNILNQEQP
SEQ ID NO: 51
ATGAGAATACCATTAATTAGCTAGGGACCAAAATTCAAAGACAAAATGGATT
TTCAGGTGCAGATTTTCAGCTTCCTGCTAATCAGTGCCTCAGTCATACTGTCC
AGAGGACAAGTTGTTCTCACCCAGTCTCCAGTAATCATGTCTGCATCTCCAGG
GGAGAAGGTCACCATGACCTGCAGTGCCAGCTCAAGTATAAGTTACATGTAC
TGGTACCAGCAGAAGCCAGGCACCTCCCCCAAAAGATGGATTTATGACACAT
CCAAACTGGCTTCTGGAGTCCCTGCTCGCTTCAGTGGCAGTGGGTCTGGGACC
TCTTATTCTCTCACAATCAGCAACATGGAGGCTGGAGATGCTGCCACTTATTA
CTGCCATCAGCGGGATTCTTACCCATGGACGTTCGGTGGAGGCACCAACCTG
GAAATCAAACGGGCTGATGCTGCACCAACTGTATCCATCTTCCCACCATCCA
GTGAGCAGTTAACATCTGGAGGTGCCTCAGTCGTGTGCTTCTTGAACAACTTC
TACCCCAAAGACATCAATGTCAAGTGGAAGATTGATGGCAGTGAACGACAAA
ATGGCGTCCTGAACAGTTGGACTGATCAGGACAGCAAAGACAGCACCTACAG
CATGAGCAGCACCCTCACGTTGACCAAGGACGAGTATGAACGACATAACAGC
TATACCTGTGAGGCCACTCACAAGACATCAACTTCACCCATTGTCAAGAGCTT
CAACAGGAATGAGTGTTAGAGACAAAGGTCCTGAGACGCCACCACCAGCTCC
CCAGCTCCATCCTATCTTCCCTTCTAAGGTCTTGGAGGCTTCCCCACAAGCGA
CCTACCACTGTTGCGGTGCTCCAAACCTCCTCCCCACCTCCTTCTCCTCCTCCT
CCCTTTCCTTGGCTTTTATCATGCTAATATTTGCAGAAAATATTCAATAAAGT
GAGTCTTTGCACTTGAAAA
SEQ ID NO: 52
MDFQVQIFSFLLISASVILSRGQVVLTQSPVIMSASPGEKVTMTCSASSSISYMYW
YQQKPGTSPKRWIYDTSKLASGVPARFSGSGSGTSYSLTISNMEAGDAATYYCH
QRDSYPWTFGGGTNLEIKRADAAPTVSIFPPSSEQLTSGGASVVCFLNNFYPKDI
NVKWKIDGSERQNGVLNSWTDQDSKDSTYSMSSTLTLTKDEYERHNSYTCEAT
HKTSTSPIVKSFNRNEC
SEQ ID NO: 53
SASSSISYMY
SEQ ID NO: 54
DTSKLAS
SEQ ID NO: 55
HQRDSYPWT
SEQ ID NO: 56
TTTTCCATCCTCTTCTCATAGAGCCTCCATCAGACCATGGCTGTCCTGGCACT
GCTCCTCTGCCTGGTGACATTCCCAAGCTGTGTCCTGTCCCAGGTGCAGCTGA
AGGAGTCAGGACCTGACCTGGTGGCGCCCTCACAGAGCCTGTCCATCACATG
CACTGTCTCAGGATTCTCATTAAGCAAATTTGGTGTAAACTGGGTTCGCCAGC
CTCCAGGAAAGGGTCTGGAGTGGCTGGGAGTAATATGGGGTGACGGGAGCA
CAAGTTATAATTCAGGTCTCATATCAAGACTGAGCATCAGCAAGGAGAACTC
CAAGAGCCAGGTTTTCTTAAAACTGAACAGTCTGCAAGCTGATGACACAGCC
ACATACTACTGTGTCAAACCGGGGGGTGACTACTGGGGTCACGGAACCTCAG
TCACCGTCTCCTCAGCCAAAACGACACCCCCATCTGTCTATCCACTGGCCCCT
GGATCTGCTGCCCAAACTAACTCCATGGTGACCCTGGGATGCCTGGTCAAGG
GCTATTTCCCTGAGCCAGTGACAGTGACCTGGAACTCTGGATCCCTGTCCAGC
GGTGTGCACACCTTCCCAGCTGTCCTGCAGTCTGACCTCTACACTCTGAGCAG
CTCAGTGACTGTCCCCTCCAGCACCTGGCCCAGCGAGACCGTCACCTGCAAC
GTTGCCCACCCGGCCAGCAGCACCAAGGTGGACAAGAAAATTGTGCCCAGG
GATTGTGGTTGTAAGCCTTGCATATGTACAGTCCCAGAAGTATCATCTGTCTT
CATCTTCCCCCCAAAGCCCAAGGATGTGCTCACCATTACTCTGACTCCTAAGG
TCACGTGTGTTGTGGTAGACATCAGCAAGGATGATCCCGAGGTCCAGTTCAG
CTGGTTTGTAGATGTGGAGGTGCACACAGCTCAGACGCAACCCCGGGAGGAG
CAGTTCAACAGCACTTTCCGCTCAGTCAGTGAACTTCCCATCATGCACCAGGA
CTGGCTCAATGGCAAGGAGTTCAAATGCAGGGTCAACAGTGCAGCTTTCCCT
GCCCCCATCGAGAAAACCATCTCCAAAACCAAAGGCAGACCGAAGGCTCCA
CAGGTGTACACCATTCCACCTCCCAAGGAGCAGATGGCCAAGGATAAAGTCA
GTCTGACCTGCATGATAACAGACTTCTTCCCTGAAGACATTACTGTGGAGTGG
CAGTGGAATGGGCAGCCAGCGGAGAACTACAAGAACACTCAGCCCATCATG
GACACAGATGGCTCTTACTTCGTCTACAGCAAGCTCAATGTGCAGAAGAGCA
ACTGGGAGGCAGGAAATACTTTCACCTGCTCTGTGTTACATGAGGGCCTGCA
CAACCACCATACTGAGAAGAGCCTCTCCCACTCTCCTGGTAAATGATCCCAG
TGTCCTTGGAGCCCTCTGGTCCTACAGGACTCTGACACCTACCTCCACCCCTC
CCTGTATAAATAAAGCACCCAGCACTGCCTTGGGACCCTGCAAAAAAAAAAA
AAAAA
SEQ ID NO: 57
MAVLALLLCLVTFPSCVLSQVQLKESGPDLVAPSQSLSITCTVSGFSLSKFGVNW
VRQPPGKGLEWLGVIWGDGSTSYNSGLISRLSISKENSKSQVFLKLNSLQADDT
ATYYCVKPGGDYWGHGTSVTVSSAKTTPPSVYPLAPGSAAQTNSMVTLGCLVK
GYFPEPVTVTWNSGSLSSGVHTFPAVLQSDLYTLSSSVTVPSSTWPSETVTCNVA
HPASSTKVDKKIVPRDCGCKPCICTVPEVSSVFIFPPKPKDVLTITLTPKVTCVVVD
ISKDDPEVQFSWFVDDVEVHTAQTQPREEQFNSTFRSVSELPIMHQDWLNGKEF
KCRVNSAAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTCSVL
HEGLHNHHTEKSLSHSPGK
SEQ ID NO: 58
SKFGVN
SEQ ID NO: 59
VIWGDGSTSYNSGLIS
SEQ ID NO: 60
CVKPGGDY
SEQ ID NO: 61
GCATAGATCTGCCACCATGGACTTTCAGGTCCAGATATTTAGCTTTCTATTGA
TTAGCGCCTCTGTCATTCTGAGTAGGGGGCAGGTGGTGCTCACCCAGTCTCCA
GTGATCATGTCAGCCTCACCAGGAGAAAAAGTGACTATGACCTGCTCAGCAT
CCTCCAGCATCAGTTACATGTACTGGTACCAGCAGAAGCCAGGCACCTCGCC
CAAGCGTTGGATCTACGATACTTCCAAGCTGGCAAGTGGGGTACCCGCACGC
TTCAGTGGAAGTGGCTCCGGAACCTCGTACAGTTTGACCATTTCAAATATGGA
AGCTGGGGACGCAGCTACATATTATTGCCACCAGAGAGACTCCTACCCGTGG
ACCTTCGGAGGCGGTACTAATTTAGAGATCAAGAGGACCGTAGCCGCTCCTT
CCGTGTTCATCTTTCCCCCTTCCGACGAACAACTGAAAAGCGGTACAGCCTCC
GTGGTTTGTCTGCTGAACAACTTCTACCCCCGGGAGGCTAAAGTTCAGTGGA
AGGTTGACAATGCTCTGCAGTCAGGCAACTCTCAAGAGAGCGTCACGGAGCA
AGATAGCAAAGATTCTACATATTCTCTCTCTTCTACACTTACACTTAGCAAGG
CCGATTATGAGAAGCACAAGGTGTATGCCTGCGAGGTGACTCATCAGGGTCT
TTCTTCTCCTGTCACTAAAAGCTTCAACCGAGGCGAATGTTGATGAAGATCTT
ACG
SEQ ID NO: 62
MDFQVQIFSFLLISASVILSRGQVVLTQSPVIMSASPGEKVTMTCSASSSISYMYW
YQQKPGTSPKRWIYDTSKLASGVPARFSGSGSGTSYSLTISNMEAGDAATYYCH
QRDSYPWTFGGGTNLEIKRTVAAPSVFIFPPSDEQLKSGTASVVCLLNNFYPREA
KVQWKVDNALQSGNSQESVTEQDSKDSTYSLSSTLTLSKADYEKHKVYACEVT
HQGLSSPVTKSFNRGEC
SEQ ID NO: 63
SASSSISYMY
SEQ ID NO: 64
DTSKLAS
SEQ ID NO: 65
HQRDSYPWT
SEQ ID NO: 66
GAGCGGTACCGCCACCATGGCAGTGCTGGCCCTTCTTCTATGTCTGGTGACCT
TCCCATCCTGCGTCCTGAGCCAGGTACAACTGAAGGAGTCGGGCCCAGACCT
AGTGGCTCCGTCACAATCACTCTCCATTACGTGCACTGTCTCCGGCTTCTCTTT
GTCTAAATTCGGCGTGAATTGGGTGCGACAGCCCCCCGGGAAGGGGCTTGAG
TGGTTAGGAGTTATCTGGGGTGACGGCTCAACCAGCTACAACTCAGGACTAA
TCTCACGCTTGTCAATTTCAAAGGAGAATTCAAAGTCTCAGGTGTTCCTTAAG
CTCAACTCGCTGCAAGCCGACGATACCGCAACCTATTACTGCGTCAAACCTG
GCGGGGACTACTGGGGCCATGGCACCTCCGTCACAGTGAGTTCCGCATCCAC
AAAGGGTCCCAGTGTTTTTCCTTTGGCGCCCTCTAGCAAATCGACATCTGGCG
GCACAGCCGCACTTGGGTGCTTGGTTAAAGACTACTTCCCCGAACCGGTGAC
AGTATCTTGGAACTCTGGCGCTCTTACCAGCGGAGTTCATACCTTCCCTGCCG
TATTACAGTCTAGCGGGCCCTACTCCCTCTCCTCTGTCGTGACAGTCCCAAGC
TCTTCTCTGGGAACTCAAACCTACATCTGCAATGTGAACCATAAACCTAGCAA
CACGAAAGTGGACAAAAAGGTCGAACCCAAGAGTTGCGACAAGACACACAC
CTGCCCTCCTTGTCCTGCTCCAGAGCTCCTCGGCGGACCTAGCGTTTTCTTGTT
CCCTCCGAAACCAAAGGACACCTTGATGATTTCTCGGACCCCCGAGGTGACA
TGTGTAGTAGTTGATGTCTCCCACGAGGACCCTGAGGTCAAGTTTAATTGGTA
TGTGGACGGTGTGGAGGTCCACAACGCCAAAACAAAACCACGGGAGGAACA
GTACAATTCCACATATAGGGTGGTGAGCGTCCTTACCGTCCTGCATCAGGATT
GGTTAAATGGTAAGGAGTATAAGTGTAAGGTGTCTAACAAGGCTCTGCCTGC
TCCCATCGAAAAAACTATAAGTAAGGCCAAAGGACAGCCCAGGGAACCTCA
GGTGTATACTCTTCCACCCAGTAGAGATGAGCTGACTAAAAACCAGGTGTCC
CTGACTTGTCTGGTGAAGGGATTTTACCCATCCGATATCGCCGTGGAATGGGA
GTCCAACGGACAGCCAGAAAACAATTATAAAACTATGCCACCAGTGCTGGAT
AGTGATGGTAGTTTTTTTCTGTACAGTAAGCTGACTGTTGATAAGAGTAGATG
GCAGCAGGGTAATGTTTTTAGTTGTAGCGTTATGCACGAAGCTCTGCACAATC
ACTATACTCAGAAGAGCCTGAGCCTGAGCCCCGGTAAGTGATGAGGTACCGA
GC
SEQ ID NO: 67
MAVLALLLCLVTFPSCVLSQVQLKESGPDLVAPSQSLSITCTVSGFSLSKFGVNW
VRQPPGKGLEWLGVIWGDGSTSYNSGLISRLSISKENSKSQVFLKLNSLQADDTA
TYYCVKPGGDYWGHGTSVTVSSASTKGPSVFPLAPSSKSTSGGTAALGCLVKDY
FPEPVTVSWNSGALTSGVHTFPAVLQSSGPYSLSSVVTVPSSSLGTQTYICNVNH
KPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTC
LVKGFYPSDIA VEWESNGQPENNYKTMPPVLDSDGSFFLYSKLTVDKSRWQQG
NVFSCSVMHEALHNHYTQKSLSLSPGK
SEQ ID NO: 68
GFSLSKFGVN
SEQ ID NO: 69
VIWGDGSTSYNSGLIS
SEQ ID NO: 70
PGGDY
SEQ ID NO: 71
AAGCTTGCCACCATGAAGTACCTGCTGCCCACCGCTGCTGCTGGCTTGCTGCT
GCTGGCAGCTCAGCCTGCCATGGCCGAGATCGTGCTGACCCAGTCTCCTGGC
ACCCTGTCTCTGAGCCCTGGCGAGAGAGCTACCCTGTCCTGCTCCGCCTCCTC
CAGCATCTCCTACATGTACTGGTATCAGCAGAAGCCCGGCCAGGCCCCTCGG
CTGCTGATCTACGATACCTCCAAGCTGGCCTCCGGCATCCCCGACAGATTCTC
CGGCTCTGGCTCTGGCACCGACTTCACCCTGACCATCTCCCGGCTGGAACCCG
AGGACTTCGCCGTGTACTACTGCCACCAGCGGGACTCCTACCCCTGGACCTTT
GGCCAGGGCACCAAGCTGGAAATCAAGCGGACCGTGGCCGCTCCCTCCGTGT
TCATCTTCCCACCTTCCGACGAGCAGCTGAAGTCCGGCACCGCTTCTGTCGTG
TGCCTGCTGAACAACTTCTACCCCCGCGAGGCCAAGGTGCAGTGGAAGGTGG
ACAACGCCCTGCAGTCCGGCAACTCCCAGGAATCCGTGACCGAGCAGGACTC
CAAGGACAGCACCTACTCCCTGTCCTCTACCCTGACCCTGTCCAAGGCCGACT
ACGAGAAGCACAAGGTGTACGCCTGCGAAGTGACCCACCAGGGCCTGTCTAG
CCCCGTGACCAAGTCTTTCAACCGGGGCGAGTGCTGATGAGGATCCTGATGA
SEQ ID NO: 72
KLATMKYLLPTAAAGLLLLAAQPAMAEIVLTQSPGTLSLSPGERATLSCSASSSIS
YMYWYQQKPGQAPRLLIYDTSKLASGIPDRFSGSGSGTDFTLTISRLEPEDFAVY
YCHQRDSYPWTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYAC
EVTHQGLSSPVTKSFNRGEC
SEQ ID NO: 73
AAGCTTGCCACCATGGACCTGCTGTGCAAGAACATGAAGCACCTGTGGTTCT
TTCTGCTGCTGGTGGCCGCTCCCAGATGGGTGCTGTCTCAGGTGCAGCTGGTG
GAATCTGGCCCTGGCCTGGTGCAGCCTTCCAGATCCCTGTCTCTGACCTGCTC
CTCCAGCGGCTTCAGCCTGTCCAAGTTCGGCGTGAACTGGGTGCGACAGCCT
CCTGGCAAGGGCCTGGAATGGGTGGGAGTGATCTGGGGCGACGGCTCCACCT
CCTACAACTCCGGCCTGATCTCCAGAGTGACCATCTCCCGGGACACCTCCAA
GAACCAGCTGTTCCTGAAGATGGACTCCCTGACCGCCGAGGACACCGCCGTG
TACTACTGTGCTAGACCTGGCGGCGACTACTGGGGCCAGGGCACAACAGTGA
CCGTGTCCTCCGCTTCCACCAAGGGCCCCTCTGTGTTTCCTCTGGCCCCCTCC
AGCAAGTCCACCTCTGGTGGAACTGCCGCTCTGGGCTGCCTCGTGAAGGACT
ACTTCCCCGAGCCCGTGACAGTGTCCTGGAACTCTGGCGCTCTGACCTCCGGC
GTGCACACCTTTCCAGCTGTGCTGCAGTCCAGCGGCCTGTACTCCCTGTCCTC
CGTCGTGACCGTGCCTTCCAGCTCTCTGGGCACCCAGACCTACATCTGCAACG
TGAACCACAAGCCCTCCAATACCAAGGTGGACAAGAAGGTGGAACCCAAGT
CCTGCGACAAGACCCACACCTGTCCCCCTTGTCCTGCCCCTGAACTGCTGGGC
GGACCTTCCGTGTTCCTGTTCCCCCCAAAGCCCAAGGACACCCTGATGATCTC
CCGGACCCCCGAAGTGACCTGCGTGGTGGTGGATGTGTCCCACGAGGACCCT
GAAGTGAAGTTCAATTGGTACGTGGACGGCGTGGAAGTGCACAACGCCAAG
ACCAAGCCTAGAGAGGAACAGTACAACTCCACCTACCGGGTGGTGTCCGTGC
TGACCGTGCTGCATCAGGACTGGCTGAACGGCAAAGAGTACAAGTGCAAGGT
GTCCAACAAGGCCCTGCCTGCCCCCATCGAAAAGACCATCAGCAAGGCTAAG
GGCCAGCCCCGCGAGCCCCAGGTGTACACACTGCCTCCATCCCGGGAAGAGA
TGACCAAGAATCAGGTGTCCCTGACCTGTCTCGTGAAAGGCTTCTACCCCTCC
GATATCGCCGTGGAATGGGAGTCCAACGGCCAGCCCGAGAACAACTACAAG
ACCACCCCCCCTGTGCTGGACTCCGACGGCTCATTCTTCCTGTACAGCAAGCT
GACAGTGGACAAGTCCCGGTGGCAGCAGGGCAACGTGTTCTCCTGCTCCGTG
ATGCACGAGGCCCTGCACAACCACTACACCCAGAAGTCCCTGTCCCTGAGCC
CCGGCAAGTGATGATGAGGATCCTGA
SEQ ID NO: 74
KLATMDLLCKNMKHLWFFLLLVAAPRWVLSQVQLVESGPGLVQPSRSLSLTCSS
SGFSLSKFGVNWVRQPPGKGLEWVGVIWGDGSTSYNSGLISRVTISRDTSKNQLF
LKMDSLTAEDTAVYYCARPGGDYWGQGTTVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 75
GCGGGGCAGCCTCACACAGAACACACACAGATATGGGTGTACCCACTCAGCT
CCTGTTGCTGTGGCTTACAGTCGTAGTTGTCAGATGTGACATCCAGATGACTCA
GTCTCCAGCTTCACTGTCTGCATCTGTGGGAGAAACTGTCACCATCACATGTG
GAGCAAGTGAGAATATTTACGGTGCTTTAAATTGGTATCAGCGGAAACAGGG
AAAATCTCCTCAGCTCCTGATTTATGGCGCAAGTAATTTGGCAGATGGCATGT
CATCGAGGTTCAGTGGCAGTGGATCTGGTAGACAGTATTCTCTCAAGATCAG
TAGCCTGCATCCTGACGATGTTGCAACGTATTACTGTCAAAATGTATTAAGTA
GTCCGTACACGTTCGGAGGGGGGACCAAGCTGGAAATAAAACGGGCTGATGC
TGCACCAACTGTATCCATCTTCCCACCATCCAGTGAGCAGTTAACATCTGGAG
GTGCCTCAGTCGTGTGCTTCTTGAACAACTTCTACCCCAAAGACATCAATGTC
AAGTGGAAGATTGATGGCAGTGAACGACAAAATGGCGTCCTGAACAGTTGGA
CTGATCAGGACAGCAAAGACAGCACCTACAGCATGAGCAGCACCCTCACGT
TGACCAAGGACGAGTATGAACGACATAACAGCTATACCTGTGAGGCCACTCA
CAAGACACCAACTTCACCCATTGTCAAGAGCTTCAACAGGAATGAGTGTTA
GAGACAAAGGTCCTGAGACGCCACCACCAGCTCCCCAGCTCCATCCTATCTTC
CCTTCTAAGGTCTTGGAGGCTTCCCCACAAGCGACTACCACTGTTGCGGTGCT
CCAAACCTCCTCCCCACCTCCTTCTCCTCCTCCTCCCTTTCCTTGGCTTTATCATG
CTAATATTTGCAGAAAATATTCAATAAAGTGATCTTTGCACAAAAAAAAAAA
AAAAAAAAAAAAAAA
SEQ ID NO: 76
LMTQSPASLSASVGETVTITCGASENIYGALNWYQRKQGKSPQLLIYGASNLAD
GMSSRFSGSGSGRQYSLKISSLHPDDVATYYCQNVLSSPYTFGGGTKLEIKKG
SEQ ID NO: 77
GASENIYGALN
SEQ ID NO: 78
GASNLAD
SEQ ID NO: 79
CQNVLSSPYT
SEQ ID NO: 80
ACGCGGGACACAGTAGTCTCTACAGTCACAGGAGTACACAGGACATTGCCAT
GGGTTGGAGCTGTATCATCTTCTTTCTGGTAGCAACAGCTACAGGTGTGCACTC
CCAGGTCCAGCTGCAGCAGTCTGGGCCTGAGGTGGTGAGGCCTGGGGTCTCAG
TGAAGATTTCCTGCAAGGGTTCCGGCTACACATTCACTGATTATGCTATGCACT
GGGTGAAGCAGAGTCATGCAAAGAGTCTCGAGTGGATTGGACTTATTAGTACT
TACAGTGGTGATACAAAGTACAACCAGAATTTAAGGGCAAGGCCACAATGACT
GTAGACAAATCCTCCAACACAGCCTATATGGAACTTGCCAGATTGACATCTGAG
GATTCTGCCATCTATTACTGTGCAAGAGGGGATTATTCCGGTAGTAGGTACTGG
TTTGCTTACTGGGGCCAAGGGACTCTGGTCACTGTCTCTGCAGCCAAAACGACA
CCCCCATCTGTCTATCCACTGGCCCCTGGATCTGCTGCCCAAACTAACTCCATGG
TGACCCTGGGATGCCTGGTCAAGGGCTATTTCCCTGAGCCAGTGACAGTGACC
TGGAACTCTGGATCCCTGTCCAGCGGTGTGCACACCTTCCCAGCTGTTCCTGCA
GTCTGACCTCTACACTCTGAGCAGCTCAGTGACTGTCCCCTCCAGCACCTGGCC
CAGCGAGACCGTCACCTGCAACGTTGCCCACCCGGCCAGCAGCACCAAGGTGG
ACAAGAAAATTGTGCCCAGGGATTGTGGTTGTAAGCCTTGCATATGTACAGTC
CCAGAAGTATCATCTGTCTTCATCTTCCCCCCAAAGCCCAAGGATGTGCTCACC
ATTACTCTGACTCCTAAGGTCACGTGTGTTGTGGTAGACATCAGCAAGGATGA
TCCCGAGGTCCAGTTCAGCTGGTTTGTAGATGATGTGGAGGTGCACACAGCTC
AGACGCAACCCCGGGAGGAGCAGTTCAACAGCACTTTCCGCTCAGTCAGTGAA
CTTCCCATCATGCACCAGGACTGGCTCAATGGCAAGGAGTTCAAATGCAGGGT
CAACAGTGCAGCTTTCCCTGCCCCCATCGAGAAAACCATCTCCAAAACCAAAG
GCAGACCGAAGGCTCCACAGGTGTACACCATTCCACCTCCCAAGGAGCAGAT
GGCCAAGGATAAAGTCAGTCTGACCTGCATGATAACAGACTTCTTCCCTGAAG
ACATTACTGTGGAGTGGCAGTGGAATGGGCAGCCAGCGGAGAACTACAAGAA
CACTCAGCCCATCATGGACACAGATGGCTCTTACTTCGTCTACAGCAAGCTCA
ATGTGCAGAAGAGCAACTGGGAGGCAGGAAATACTTTCACCTGCTCTGTGTTA
CATGAGGGCCTGCACAACCACCATACTGAGAAGAGCCTCTCCCACTCTCCTGGT
AAATGATCCCAGTGTCCTTGGAGCCCTCTGGCCCTACAGGACTTTGACACCTAC
CTCCACCCCTCCCTGTATAAATAAAGCACCCAGCACTGCCTCGGGACCCTGCAT
AAAAAAAAAAAAAAAAAAAAAAAAAAAA
SEQ ID NO: 81
LEESGPEVVRPGVSVKISCKGSGYTFTDYAMHWVKQSHAKSLEWIGLISTYSG
DTKYNQNFKGKATMTVDKSSNTAYMELARLTSEDSAIYYCARGDYSGSRYW
FAYWGQGTTVTR
SEQ ID NO: 82
GYTFTDYAMH
SEQ ID NO: 83
LISTYSGDTKYNQNFKG
SEQ ID NO: 84
GDYSGSRYWFAY
SEQ ID NO: 85
DIQMTQSPSSLSASVGDRVTITCGASENIYGALNWYQRKPGKSPKLLIYGASNLAD
GMPSRFSGSGSGRQYTLTISSLQPEDVATYYCQNVLSSPYTFGGGTKLEIK
SEQ ID NO: 86
DIQMTQSPSSLSASVGDRVTITCGASENIYGALNWYQRKPGKSPKLLIYGASNLAD
GMPSRFSGSGSGRQYTLTISSLQPEDVATYYCQNVLSSPYTFGGGTKLEIK
SEQ ID NO: 87
DIQMTQSPSSLSASVGDRVTITCQASENIYGALNWYQRKPGKSPKLLIYGASNLAT
GMPSRFSGSGSGTDYTFTISSLQPEDIATYYCQNVLSSPYTFGGGTKLEIK
SEQ ID NO: 88
DIQMTQSPSSLSASVGDRVTITCQASENIYGALNWYQRKPGKSPKLLIYGASNLAT
GMPSRFSGSGSGRQYTFTISSLQPEDIATYYCQQVLSSPYTFGGGTKLEIK
SEQ ID NO: 89
DIQMTQSPSSLSASVGDRVTITCCASENIYGALNWYQRKPGKSPNLLIYGASNLAD
GMPSRFSGSGSGRQYTLTISSLQPEDVATYYCQNVLSSPYTFGGGTKLEIK
SEQ ID NO: 90
QVQLQQSGPEVVRPGVSVKISCKGSGYTFTDYAMHWVKQSHAKSLEWIGLISTYSGD
TKYNQNFKGKATMTVDKSASTAYMELSSLRSEDTAVYYCARGDYSGSRYWFAYWG
QGTLVTVSS
SEQ ID NO: 91
QVQLQQSGPEVVRPGVSVKISCKGSGYTFTDYAMHWVKQSHAKSLEWIGLISTYSGD
TKYNQNFKGKATMTVDTSISTAYMELSRLRSDDTAIYYCARGDYSGSRYWFAYWGQ
GTLVTVSS
SEQ ID NO: 92
QVQLQQSGPEVVRPGVSVKISCKGSGYTFTDYAMHWVKQSHAKSLEWIGLISTYSGD
TKYSQKFQGRVTMTVDKSASTAYMELSSLRSEDTAVYYCARGDYSGSRYWFAYWG
QGTLVTVSS
SEQ ID NO: 93
QVQLQQSGPEVVRPGVSVKISCKGSGYTFTDYAMHWVKQSHAKSLEWIGLISTYSGD
TKYNQKFQGKATMTVDKSASTAYMELSSLRSEDTAVYYCARGDYSGSRYWFAYWG
QGTLVTVSS
SEQ ID NO: 94
QVQLQQSGPEVVRPGVSVKISCKGSGYTFTDYAMHWVKQSHAKSLEWIGLISTYSGD
TKYNQNFKGKATMTVDKSTSTAYMELSSLRSEDTAVYYCARGDYSGSRYWFAYWG
QGTLVTVSS
SEQ ID NO: 95
ATGGGCGTGCCCACCCAGCTGCTGCTGCTGTGGCTGACCGTGGTGGTGGTGC
GGTGCGACATCCAGATGACCCAGTCCCCTAGCTCTCTGAGCGCCTCCGTGGG
CGACAGGGTGACCATCACCTGCCAAGCCTCTGAGAACATCTACGGCGCCCTG
AACTGGTACCAGAGGAAGCCCGGCAAGAGCCCCAAGCTGCTGATCTACGGC
GCCTCTAACCTGGCCACCGGCATGCCTAGCCGGTTCTCCGGCTCCGGCAGCG
GCACCGACTACACCTTCACCATCTCCTCCCTGCAACCCGAGGACATCGCCACC
TACTACTGCCAGCAGGTGCTGTCCTCCCCCTACACCTTCGGCGGCGGCACCAA
ACTGGAGATCAAGCGGACCGTGGCCGCCCCCAGCGTGTTCATCTTCCCCCCCT
CTGACGAGCAGCTGAAGTCCGGCACCGCCTCTGTGGTGTGCCTGCTGAACAA
CTTCTACCCCAGGGAGGCCAAGGTCCAGTGGAAGGTGGACAACGCCCTGCAG
TCCGGCAACAGCCAGGAGTCTGTGACCGAGCAGGACTCCAAGGACTCCACCT
ACAGCCTGTCTAGCACCCTGACCCTGTCCAAGGCCGACTACGAGAAGCACAA
GGTGTACGCCTGCGAGGTGACCCACCAGGGCCTGTCCAGCCCTGTGACCAAG
TCCTTCAACAGGGGCGAGTGCTGA
SEQ ID NO: 96
MGVPTQLLLLWLTVVVVRCDIQMTQSPSSLSASVGDRVTITCQASENIYGALNWY
QRKPGKSPKLLIYGASNLATGMPSRFSGSGSGTDYTFTISSLQPEDIATYYCQQVLS
SPYTFGGGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVT
KSFNRGEC
SEQ ID NO: 97
QASENIYGALN
SEQ ID NO: 98
GASNLAT
SEQ ID NO: 99
QQVLSSPYT
SEQ ID NO: 100
ATGGGCTGGTCCTGCATCATCTTCTTCCTGGTGGCCACCGCCACCGGCGTGCA
CAGCCAGGTGCAGCTTGTGCAGAGCGGCGCCGAGGTGAAGAAGCCCGGCGC
CAGCGTGAAGGTGTCCTGCAAGGCCTCCGGCTACACCTTCACCGACTACGCC
ATGCACTGGGTGCGGCAGGCCCCCGGCCAGCGGCTGGAGTGGATGGGCCTGA
TCAGCACCTACTCTGGCGACACCAAGTACAACCAGAACTTCCAGGGCCGGGT
GACCATGACCGTGGACAAGAGCGCCAGCACCGCCTACATGGAGCTGTCCTCC
CTGAGGTCTGAGGACACCGCCGTGTACTACTGCGCCCGGGGCGACTACAGCG
GCAGCCGGTACTGGTTCGCCTACTGGGGCCAGGGCACCCTGGTGACCGTGTC
CAGCGCCTCTACCAAGGGCCCCAGCGTGTTTCCCCTGGCCCCTTCCTCCAAAA
GCACCAGCGGCGGTACCGCCGCCCTGGGCTGCCTGGTGAAGGACTACTTCCC
CGAGCCCGTGACCGTGAGCTGGAACTCCGGCGCCCTGACCAGCGGCGTGCAC
ACCTTCCCTGCCGTGCTGCAAAGCTCCGGCCTGTACTCCCTGAGCTCTGTGGT
GACCGTGCCCTCCAGCTCCCTGGGCACCCAGACCTACATCTGCAACGTGAAC
CACAAGCCCAGCAACACCAAGGTGGACAAGAAGGTGGAGCCTAAGTCTTGC
GACAAGACCCACACCTGCCCCCCTTGCCCTGCCCCTGAGCTGCTGGGCGGCC
CCAGCGTGTTCCTGTTCCCTCCCAAGCCCAAGGACACCCTGATGATCTCCCGG
ACCCCTGAGGTGACCTGCGTGGTGGTGGATGTGAGCCACGAGGATCCTGAAG
TGAAGTTCAATTGGTATGTGGATGGCGTGGAGGTGCACAACGCCAAGACCAA
GCCCCGGGAGGAGCAGTACAACAGCACCTACAGGGTGGTGTCCGTGCTGACC
GTGCTGCACCAGGACTGGCTGAACGGCAAGGAGTACAAGTGCAAGGTGTCCA
ACAAGGCCCTGCCCGCCCCCATCGAGAAGACCATCTCCAAGGCCAAGGGCCA
GCCCCGGGAGCCCCAGGTGTACACCCTGCCTCCCAGCCGGGACGAGCTGACC
AAGAACCAGGTGTCCCTGACCTGCCTGGTGAAGGGCTTCTACCCCTCTGACA
TCGCCGTGGAGTGGGAGAGCAACGGCCAGCCCGAGAACAACTACAAGACCA
CCCCCCCCGTGCTGGACTCCGACGGCTCCTTCTTCCTGTACTCTAAGCTGACC
GTGGACAAGTCCCGCTGGCAGCAGGGCAACGTGTTCAGCTGCTCCGTGATGC
ACGAGGCCCTGCACAACCACTACACCCAGAAGAGCCTGAGCCTGTCTCCCGG
CAAGTGA
SEQ ID NO: 101
MGWSCIIFFLVATATGVHSQVQLVQSGAEVKKPGASVKVSCKASGYTFTDYAMH
WVRQAPGQRLEWMGLISTYSGDTKYNQNFQGRVTMTVDKSASTAYMELSSLRSE
DTAVYYCARGDYSGSRYWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGN
VFSCSVMHEALHNHYTQKSLSLSPGK
SEQ ID NO: 102
GYTFTDYAMH
SEQ ID NO: 103
ISTYSGDTKYNQNFQG
SEQ ID NO: 104
GDYSGSRYWFAY
SEQ ID NO: 105
MKYLLPTAAAGLLLLAAQPAMAEIVMTQSPLSLPVSPGEPASISCKASQSVSNDV
AWYLQKPGQSPKLLIYYASNRYTGVPDRFSGSGSGTDFTLKISRVEAEDLGVYY
CQQDYSSPLTFGQGTKLEIKRTVAAPSVFIFPPSDEQLKSGTASVVCLLNNFYPRE
AKVQWKVDNALQSGNSQESVTEQDSKDSTYSLSSTLTLSKADYEKHKVYACEV
THQGLSSPVTKSFNRGEC
SEQ ID NO: 106
MDWTWRILFLVAAATGAQAQVQLVQSGPELKKPGASVKVSCKASGYTFTNYG
MNWVRQAPGKGLEWMGWINTYTGEPTYADDFKGRFSMSLDTSTSTAYLQISSL
KSEDTAVYFCARAYYGKYFDYWGQGTLVTVSSASTKGPSVFPLAPSSKSTSGGT
AALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 107
AAGCTTGCCACCATGAAGTACCTGCTGCCCACCGCTGCTGCTGGCTTGCTGCT
GCTGGCAGCTCAGCCTGCCATGGCCGAGATCGTGATGACCCAGTCCCCTCTG
TCCCTGCCTGTGTCTCCTGGCGAGCCTGCCTCCATCTCCTGCAAGGCCTCCCA
GTCCGTGTCCAACGACGTGGCCTGGTATCTGCAGAAGCCTGGCCAGTCCCCC
AAGCTGCTGATCTACTACGCCTCCAACCGGTACACCGGCGTGCCCGACAGAT
TCTCCGGCTCTGGCTCTGGCACCGACTTCACCCTGAAGATCTCCCGGGTGGAA
GCCGAGGACCTGGGCGTGTACTACTGTCAGCAGGACTACTCCTCCCCCCTGA
CCTTTGGCCAGGGCACCAAGCTGGAAATCAAGCGGACCGTGGCCGCTCCCTC
CGTGTTCATCTTCCCACCTTCCGACGAGCAGCTGAAGTCCGGCACCGCTTCTG
TCGTGTGCCTGCTGAACAACTTCTACCCCCGCGAGGCCAAGGTGCAGTGGAA
GGTGGACAACGCCCTGCAGTCCGGCAACTCCCAGGAATCCGTGACCGAGCAG
GACTCCAAGGACAGCACCTACTCCCTGTCCAGCACCCTGACCCTGTCCAAGG
CCGACTACGAGAAGCACAAGGTGTACGCCTGCGAAGTGACCCACCAGGGCCT
GTCTAGCCCCGTGACCAAGTCTTTCAACCGGGGCGAGTGCTGATGATGAGGA
TCCTGA
SEQ ID NO: 108
KLATMKYLLPTAAAGLLLLAAQPAMAEIVMTQSPLSLPVSPGEPASISCKASQSV
SNDVAWYLQKPGQSPKLLIYYASNRYTGVPDRFSGSGSGTDFTLKISRVEAEDLG
VYYCQQDYSSPLTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYA
CEVTHQGLSSPVTKSFNRGEC
SEQ ID NO: 109
AAGCTTGCCACCATGGACTGGACCTGGCGCATCCTGTTTCTGGTGGCCGCTGC
TACAGGCGCCCAGGCTCAGGTGCAGCTGGTGCAGTCTGGACCCGAGCTGAAG
AAACCTGGCGCCTCCGTGAAGGTGTCCTGCAAGGCTTCCGGCTACACCTTTAC
CAACTACGGCATGAACTGGGTGCGACAGGCCCCTGGCAAGGGCCTGGAATG
GATGGGCTGGATCAACACCTACACCGGCGAGCCCACCTACGCCGACGACTTC
AAGGGCCGGTTCTCCATGTCCCTGGACACCTCCACCAGCACCGCCTACCTGC
AGATCTCCAGCCTGAAGTCCGAGGATACCGCCGTGTACTTCTGCGCCAGAGC
CTACTACGGCAAGTACTTCGACTACTGGGGCCAGGGCACCCTCGTGACCGTG
TCCTCTGCTTCTACCAAGGGCCCCTCCGTGTTCCCTCTGGCCCCTTCCAGCAA
GTCTACCTCTGGCGGCACAGCCGCTCTGGGCTGCCTCGTGAAGGACTACTTCC
CCGAGCCCGTGACAGTGTCTTGGAACTCTGGCGCCCTGACCTCCGGCGTGCA
CACCTTTCCAGCTGTGCTGCAGTCCTCCGGCCTGTACTCCCTGTCCTCCGTCGT
GACTGTGCCCTCCAGCTCTCTGGGCACCCAGACCTACATCTGCAACGTGAAC
CACAAGCCCTCCAACACCAAGGTGGACAAGAAGGTGGAACCCAAGTCCTGC
GACAAGACCCACACCTGTCCCCCTTGTCCTGCCCCTGAACTGCTGGGCGGAC
CCAGCGTGTTCCTGTTCCCCCCAAAGCCCAAGGACACCCTGATGATCTCCCGG
ACCCCCGAAGTGACCTGCGTGGTGGTGGATGTGTCTCACGAGGACCCTGAAG
TGAAGTTCAATTGGTACGTGGACGGCGTGGAAGTGCACAACGCCAAGACCAA
GCCTAGAGAGGAACAGTACAACTCCACCTACCGGGTGGTGTCCGTGCTGACC
GTGCTGCACCAGGATTGGCTGAACGGCAAAGAGTACAAGTGCAAGGTGTCCA
ACAAGGCCCTGCCTGCCCCCATCGAAAAGACCATCTCCAAGGCCAAGGGCCA
GCCCCGGGAACCCCAGGTGTACACACTGCCCCCTAGCAGGGACGAGCTGACC
AAGAACCAGGTGTCCCTGACCTGTCTCGTGAAAGGCTTCTACCCCTCCGATAT
CGCCGTGGAATGGGAGTCCAACGGCCAGCCTGAGAACAACTACAAGACCAC
CCCCCCTGTGCTGGACTCCGACGGCTCATTCTTCCTGTACAGCAAGCTGACAG
TGGACAAGTCCCGGTGGCAGCAGGGCAACGTGTTCTCCTGCTCCGTGATGCA
CGAGGCCCTGCACAACCACTACACCCAGAAGTCCCTGTCCCTGAGCCCCGGC
AAGTGATGATGAGGATCCTGA
SEQ ID NO: 110
KLATMDWTWRILFLVAAATGAQAQVQLVQSGPELKKPGASVKVSCKASGYTFT
NYGMNWVRQAPGKGLEWMGWINTYTGEPTYADDFKGRFSMSLDTSTSTAYLQI
SSLKSEDTAVYFCARAYYGKYFDYWGQGTLVTVSSASTKGPSVFPLAPSSKSTS
GGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 111
AACCTGTGGGGACATTGTGATGTCACAGTCTCCATCCTCCCTGGCTGTGTCAG
CAGGAGAAAAGGTCACTTTGAACTGCAAATCCAGTCAGAGTCTGCTCAACAG
TAGAACCCGAAAGAACTACTTGGCTTGGTACCAGCAAAAACCAGGGCAGTCT
CCTAAATTACTGATCTACTGGGCATCCACTAGGGAATCTGGGGTCCCTGATCG
CTTCACAGGCAGTGGATCTGGGACAGATTTCACTCTCACCATCAACAGTGTGC
AGGCTGAAGACCTGGCAGTTTATTACTGCAAGCAATCTTATAATCTCTTCACG
TTCGGCTCGGGGACAAAGTMGAAGTAAAACGGGCTGATGCTGCACCAACTGT
ATCCATCTTCCCACCATCCAGTGAGCAGTTAACATCTGGAGGTGCCTCAGTCG
TGTGCTTCTTGAACAACTTCTACCCCAAAGACACCAATGTCAAGTGGAAGATT
GATGGCAGTGAACGACAAAATGGCGTCCTGAACAGTTGGACTGATCAGGACA
GCAAAGACAGCACCTACAGCATGAGCAG
SEQ ID NO: 112
TCGDIVMSQSPSSLAVSAGEKVTLNCKSSQSLLNSRTRKNYLAWYQQKPGQSP
KLLIYWASTRESGVPDRFTGSGSGTDFTLTINSVQAEDLAVYYCKQSYNLFTFGS
GTKLEVKRADAAPTVSIFPPSSEQLTSGGASVVCFLNNFYPKDTNVKWKIDGSER
QNGVLNSWTDQDSKDSTYSMS
SEQ ID NO: 113
QSLLNSRTRKNY
SEQ ID NO: 114
WAS
SEQ ID NO: 115
KQSYNLFT
SEQ ID NO: 116
TGAGGTGCAGCTGGAGGAGTCTGGAGCTGAACTGGCGAGGCCCGGGGCTTCA
GTGAAGCTGTCTTGTAAGGCTTCTGGCTACTCCTTCACTGACTATTATATAAA
TTGGGTGAAGCAGAGGACTGGACAGGGCCTTGAGTGGATTGGAGAAATTTAT
CCTTTAGGTGGTACTAGTTTCTACAATGAGAGGTTCAAGGACAAGGCCACAC
TGACTGCAGACAAATCCTCCAGCACAGTCTACATGGAACTCAGCAGCCTGAC
ATCTGAGGACTCGGCAGTCTATTTCTGTGCAAGAGGGGATAATTATTACGAC
GTCTACTTTGACTACTGGGGCCAAGGGACCACGGTCAC
SEQ ID NO: 117
EVQLEESGAELARPGASVKLSCKASGYSFTDYYINWVKQRTGQGLEWIGEIYPL
GGTSFYNERFKDKATLTADKSSSTVYMELSSLTSEDSAVYFCARGDNYYDVYF
DYWG
SEQ ID NO: 118
GYSFTDYY
SEQ ID NO: 119
IYPLGGTS
SEQ ID NO: 120
ARGDNYYDVYFDY
SEQ ID NO: 121
AGAUGUGCCUCAACUACGAtt
SEQ ID NO: 122
UCGUAGUUGAGGCACAUCUtg
SEQ ID NO: 123
GCUCUGGAACGUGAGCAUAtt
SEQ ID NO: 124
UAUGCUCACGUUCCAGAGCcg
SEQ ID NO: 125
GCGUGCUCGUCGACAACUAtt
SEQ ID NO: 126
UAGUUGUCGACGAGCACGCgg
SEQ ID NO: 127
AGAACUCAGUGAGUGCAAAtt
SEQ ID NO: 128
UUUGCACUCACUGAGUUCUgg
SEQ ID NO: 129
GGAACGAUGCAGGAUCCUAtt
SEQ ID NO: 130
UAGGAUCCUGCAUCGUUCCtt

What is claimed is:

1-144. (canceled)

145. A method of identifying a synergistic combination for potential treatment of a subject with a cancer that expresses the 16C3 antigen comprising an antibody specific for a cancer-associated antigen (“anti-CAA antibody”), wherein said cancer-associated antigen is 16C3 and second therapeutic agent, comprising:
(i) administering to a non-human animal which comprises a tumor xenograft that expresses the 16C3 antigen, said anti-CAA antibody wherein said antibody comprises a NEO-201 antibody comprising a light chain variable region containing the complementarity determining region (CDR) 1 of SEQ ID NO: 77 or 97, CDR2 of SEQ ID NO: 78 or 98, and CDR3 of SEQ ID NO: 79 or 99, and comprising a heavy chain variable region containing the CDR1 of SEQ ID NO: 82 or 102, CDR2 of SEQ ID NO: 83 or 103, and CDR3 of SEQ ID NO: 84 or 104; and said second therapeutic agent, wherein said antibody and second therapeutic agent are administered in the same or different compositions, in either order, and
(ii) determining wherein the combined administration of said anti-CAA antibody and said second therapeutic agent results in the efficacious dosage of said anti-CAA antibody or said second therapeutic agent being lower compared to when said anti-CAA antibody or said second therapeutic agent are administered individually,
(iii) if the efficacious dosage of said anti-CAA antibody or said second therapeutic agent being lower compared to when said anti-CAA antibody or said second therapeutic agent are administered individually in step (ii) identifying the combination as a synergistic combination suitable for treating a human subject with a cancer that expresses an antigen specifically bound by said anti-CAA antibody.

146. A method of identifying a synergistic combination for potential treatment of a subject with a cancer that expresses the 16C3 antigen comprising an antibody specific for a cancer-associated antigen (“anti-CAA antibody”), wherein said cancer-associated antigen is 16C3 and second therapeutic agent which comprises an anti-cancer agent for treatment of a subject with a cancer that expresses the 16C3 antigen, comprising:
(i) contacting in vitro a composition comprising tumor cells which express the 16C3 antigen with said anti-CAA antibody, wherein said antibody comprises a NEO-201 antibody comprising a light chain variable region containing the complementarity determining region (CDR) 1 of SEQ ID NO: 77 or 97, CDR2 of SEQ ID NO: 78 or 98, and CDR3 of SEQ ID NO: 79 or 99, and comprising a heavy chain variable region containing the CDR1 of SEQ ID NO: 82 or 102, CDR2 of SEQ ID NO: 83 or 103, and CDR3 of SEQ ID NO: 84 or 104; and said second therapeutic agent, wherein said antibody and second therapeutic agent are added in combination or separately, and in either order, and
(ii) determining wherein the combined addition of said anti-CAA antibody and said second therapeutic agent results in the cytotoxic dosage of said anti-CAA antibody or said second therapeutic agent being lower compared to when said anti-CAA antibody or said second therapeutic agent are added individually to said composition comprising tumor cells which express the 16C3 antigen,
(iii) if the cytotoxic dosage of said anti-CAA antibody or said second therapeutic agent are lower compared to when said anti-CAA antibody or said second therapeutic agent are added individually to the composition in step (ii) identifying the combination as a synergistic combination potentially suitable for treating a human subject with a cancer that expresses an antigen specifically bound by said anti-CAA antibody.

147. A method of treatment, comprising administering to a subject in need thereof a synergistic combination identified according to claim 145, wherein said anti-CAA antibody and said second therapeutic agent are administered at a lower dosage than the effective dosage of said anti-CAA antibody or said second therapeutic agent when administered individually, wherein said method is for the treatment of a cancer that expresses an antigen specifically bound by said anti-CAA antibody.

148. A method of treatment, comprising administering to a subject in need thereof a synergistic combination identified according to claim 146, wherein said anti-CAA antibody and said second therapeutic agent are administered at a lower dosage than the effective dosage of said anti-CAA antibody or said second therapeutic agent when administered individually, wherein said method is for the treatment of a cancer that expresses an antigen specifically bound by said anti-CAA antibody.

149. The method of claim 145, wherein said second therapeutic agent targets the extrinsic, intrinsic and/or common apoptotic pathway.

150. The method of claim 146, wherein said second therapeutic agent targets the extrinsic, intrinsic and/or common apoptotic pathway.

151. The method of claim 145, wherein said second therapeutic agent said second therapeutic agent enhances ADCC of cancer cells.

152. The method of claim 146, wherein said second therapeutic agent said second therapeutic agent enhances ADCC of cancer cells.

153. The method of claim 145, wherein the second therapeutic agent comprises gemcitabine, cisplatin, a death receptor (DR) agonist, DR ligand or fragment or variant thereof, TRAIL, a Fas ligand, TNF receptor 1 (TNFRI), TRAIL-R2, DR4, DR5, or CD95 (APO-1/Fas), an agent that agent activates a death receptor or sensitizes said death receptor to activation by another agent.

154. The method of claim 146, wherein the second therapeutic agent comprises gemcitabine, cisplatin, a death receptor (DR) agonist, DR ligand or fragment or variant thereof, TRAIL, a Fas ligand, TNF receptor 1 (TNFR1), TRAIL-R2, DR4, DR5, or CD95 (APO-1/Fas), an agent that agent activates a death receptor or sensitizes said death receptor to activation by another agent.

155. The method of claim 145, wherein the second therapeutic agent comprises
(i) an extrinsic pathway agent that agonizes the extrinsic apoptotic pathway in cancer cells, an extrinsic pathway agent that targets PML-RARα, DR4 (TRAIL R1), DR5 (TRAIL R2), an extrinsic pathway agent that comprises TRAIL polypeptide or an agonistic fragment thereof, a Dr4 agonist, a Dr5 agonist, all trans retinoic acid (ATRA), a Dr4 or Dr5 agonist comprising an agonistic anti-Dr4 or anti-Dr5 monoclonal antibody, optionally selected from Apomab, HGS-ETR1, HGS-ETR2, and GS-TR2J, an extrinsic pathway agent comprising necrosis factor alpha (TNF-alpha), tumor necrosis factor beta (TNF-beta, lymphotoxin alpha), lymphotoxin beta (LT-beta), TRAIL (Apo2L), CD95 (Fas, APO-I) ligand, TRAMP (DR3, Apo-3) ligand, DR4 ligand, DR6 ligand or a fragment, variant, or derivative thereof, an extrinsic pathway agent comprising an anti-CD95 antibody, anti-TRAIL-R1 (DR4) antibody, anti-TRAIL-R2 (DR5) antibody, anti-DR6 antibody, anti TNF-R1/2 antibody and anti-TRAMP (DR3) antibody or a fragment or derivative thereof, an extrinsic pathway agent comprising a chemotherapeutic agent, mapatumumab (HGS-ETR1), lexatumumab (HGS-ETR2), conatumumab (AMG655), dulanermin (AMG 951, APO2L/TRAIL, PRO1762, RG3639, rhApo2L/TRAIL), tigatuzumab (CS1008), TRAIL R (DR4-Specific Altrimer, Anaphore), HGS TR2J, LBY135, drozitumab (PR085780, apomab), SL231, SM164 with TRAIL R2, or TAS266, an extrinsic pathway agent comprising doxorubicin, etoposide, cisplatin, bleomycin, 5-fluorouracil, mitomycin C, oxaliplatin, 2-deoxy-D-glucose, a platin, a drug that targets a Fas pathway or a c-FLIP pathway, 4-(4-Chloro-2-methylphenoxy)-N-hydroxybutanamide (CMH), estradiol (E2), or delta-tocotrienol;
(ii) an intrinsic pathway agent that agonizes the intrinsic apoptotic pathway in cancer cells, an agonist of a proapoptotic Bcl-2 family member, an agonist of Bax, Bak, Bad, Bcl-Xs, Bid, Bik, Bim, or Hrk, an antagonist of an antiapoptotic Bcl-2 family member, an antagonist of Bcl-2, Bcl-XL, Bcl-W, Bfl-1, or Mcl-1, an agent that targets Bcl-1, Bcl-XL, Bax, BCL-Xs and/or PML-RARα, an agent that acts directly on the mitochondrial inner membrane, and agent that antagonizes the antiapoptotic members of the Bcl-2 protein family, or an agent that enhances the activity of the proapoptotic members of the Bcl-2 family of proteins such as Bax, arsenic trioxide, lonidamine an antisense agent targeting Bcl-1, Bcl-XL, Bax, BCL-Xs, both Bcl-2 and Bcl-XL, clusterin, or comprises oblimersen sodium, a small molecule, a small molecule that recognizes the surface pocket of Bcl-2 or Bcl-XL, Antimycin-A, HA14-1, synthetic BH3 organic peptides, or a derivative thereof, farnesyl-thiosalicylic acid (FTS), estradiol (E2), delta-tocotrienol, salinomycin, or curcumin, an antimetabolite, alkylator, corticosteroid, radiation, monoclonal antibody, platin or PARP inhibitor, epirubicin, cisplatin, dacarbazine, fludarabine/cyclophosphamide, dexamethasone, or doxorubicin, FTS, CMH, TMS, or estradiol (E2);
(iii) a cytostatic agent, cytocidal agent, actinomycin D, adriamycin, arsenic trioxide, asparaginase, bleomycin, busulfan, camptosar, carboplatinum, carmustine, chlorambucil, cisplatin, corticosteroids, colicheamicin, cyclophosphamide, daunorubicin, docetaxel, doxorubicin, epirubicin, etoposide, fludarabine, fluorouracil, gemcitabina, gemcitabine, gemzar, hydroxyurea, idarubicin, ifosfamide, irinotecan, lomustine, melphalan, mercaptomurine, methotrexate, mitomycin, mitoxantrone, oxaliplatin, paclitaxel, platinol, platinex, procarbizine, raltitrexeel, rixin, steroids, streptozocin, taxol, taxotere, thioguanine, thiotepa, tomudex, topotecan, treosulfan, trihydrate, vinblastine, vincristine, vindesine, vinorelbina, vinorelbine, duanomycin, dactinomysin, esorubisin, mafosfamide, cytosine arabinoside, bis-chloroethylnitrosurea, mitomycin C, mithramycin, prednisone, hydroxyprogesterone, testosterone, tamoxifen, dacarbazine, hexamethylmelamine, pentamethylmelamine, amsacrine, chlorambudil, methylcyclohexylnitrosurea, nitrogen mustards, cyclophosphamide, 6-mercaptopurine, 6-thioguanine, cytarabine, 5-azacytidine, deoxyco-formycin, 4-hydroxyperoxycyclophosphoramide, 5-fluorouracil (5-FU), 5-fluorodeoxyuridine (5-FUdR), colchicine, trimetrexate, teni-poside, diethylstilbestrol, a DNA damaging agent, nucleophosmin, an agent which induces cellular damage as part of an enhanced or synergistic process with another agent, a catalytic antibody, prodrugs, CHK1/2 inhibitor, CBP-501, AZD7762, histone deacetylase inhibitor, vorinostat, tumour necrosis factor related apoptosis inducing ligand, BH3 mimetic, ABT737, small molecule inhibitors, tyrosine kinase inhibitors, imatinib mesylate, gefitinib, erlotinib, monoclonal antibodies, rituximab, trastuzumab, a caspase activator, apoptin, survivin, an endocrine therapeutic, a biologic response modifier, interferon, interleukin, antibody, aptamer, siRNA, oligonucleotide, enzyme, ion channel and receptor inhibitor or activator, hyperthermia, cryotherapy, agent to attenuate any adverse effects, or antiemetic, an alkylating drug, mechlorethamine, chlorambucil, Cyclophosphamide, Melphalan, Ifosfamide, antimetabolite, Methotrexate, purine antagonist, pyrimidine antagonist, 6-Mercaptopurine, 5-Fluorouracil, Cytarabile, Gemcitabine, spindle poison, Vinblastine, Vincristine, Vinorelbine, Paclitaxel, podophyllotoxin, Etoposide, Irinotecan, Topotecan, antibiotic, doxorubicin, Bleomycin, Mitomycin, nitrosoureas, Carmustine, Lomustine, inorganic ion, Cisplatin, Carboplatin, enzyme, Asparaginase, hormone, Tamoxifen, Leuprolide, Flutamide, Megestrol, an agent that targets p53, p53 pathway members, IκB kinase, IKKβ, the proteasome/ubiquitin pathway, the 20S proteasome, the PI3K/Akt pathway, or mTOR, ONY-015, INGN201, PS1145, Bortezomib, CCI779, RAD-001, an siRNA targeting MDM2, mixed lineage kinase domain like (MLKL) protein, rapamycin (RAP) or derivatives and/or analogs thereof, everolimus or RAD001; CCI-779, ABT578, SAR543, ascomycin (an ethyl analog of FK506), AP23573, AP23841, KU-0063794, INK-128, EX2044, EX3855, EX7518, compounds that bind to the ATP-binding cleft of mTOR, AZD08055, OSIO27.

156. The method of claim 146, wherein the second therapeutic agent comprises
(i) an extrinsic pathway agent that agonizes the extrinsic apoptotic pathway in cancer cells, an extrinsic pathway agent that targets PML-RARα, DR4 (TRAIL R1), DR5 (TRAIL R2), an extrinsic pathway agent that comprises TRAIL polypeptide or an agonistic fragment thereof, a Dr4 agonist, a Dr5 agonist, all trans retinoic acid (ATRA), a Dr4 or Dr5 agonist comprising an agonistic anti-Dr4 or anti-Dr5 monoclonal antibody, optionally selected from Apomab, HGS-ETR1, HGS-ETR2, and GS-TR2J, an extrinsic pathway agent comprising necrosis factor alpha (TNF-alpha), tumor necrosis factor beta (TNF-beta, lymphotoxin alpha), lymphotoxin beta (LT-beta), TRAIL (Apo2L), CD95 (Fas, APO-I) ligand, TRAMP (DR3, Apo-3) ligand, DR4 ligand, DR6 ligand or a fragment, variant, or derivative thereof, an extrinsic pathway agent comprising an anti-CD95 antibody, anti-TRAIL-R1 (DR4) antibody, anti-TRAIL-R2 (DR5) antibody, anti-DR6 antibody, anti TNF-R1/2 antibody and anti-TRAMP (DR3) antibody or a fragment or derivative thereof, an extrinsic pathway agent comprising a chemotherapeutic agent, mapatumumab (HGS-ETR1), lexatumumab (HGS-ETR2), conatumumab (AMG655), dulanermin (AMG 951, APO2L/TRAIL, PRO1762, RG3639, rhApo2L/TRAIL), tigatuzumab (CS1008), TRAIL R (DR4-Specific Altrimer, Anaphore), HGS TR2J, LBY135, drozitumab (PR085780, apomab), SL231, SM164 with TRAIL R2, or TAS266, an extrinsic pathway agent comprising doxorubicin, etoposide, cisplatin, bleomycin, 5-fluorouracil, mitomycin C, oxaliplatin, 2-deoxy-D-glucose, a platin, a drug that targets a Fas pathway or a c-FLIP pathway, 4-(4-Chloro-2-methylphenoxy)-N-hydroxybutanamide (CMH), estradiol (E2), or delta-tocotrienol;
(ii) an intrinsic pathway agent that agonizes the intrinsic apoptotic pathway in cancer cells, an agonist of a proapoptotic Bcl-2 family member, an agonist of Bax, Bak, Bad, Bcl-Xs, Bid, Bik, Bim, or Hrk, an antagonist of an antiapoptotic Bcl-2 family member, an antagonist of Bcl-2, Bcl-XL, Bcl-W, Bfl-1, or Mcl-1, an agent that targets Bcl-1, Bcl-XL, Bax, BCL-Xs and/or PML-RARα, an agent that acts directly on the mitochondrial inner membrane, and agent that antagonizes the antiapoptotic members of the Bcl-2 protein family, or an agent that enhances the activity of the proapoptotic members of the Bcl-2 family of proteins such as Bax, arsenic trioxide, lonidamine an antisense agent targeting Bcl-1, Bcl-XL, Bax, BCL-Xs, both Bcl-2 and Bcl-XL, clusterin, or comprises oblimersen sodium, a small molecule, a small molecule that recognizes the surface pocket of Bcl-2 or Bcl-XL, Antimycin-A, HA14-1, synthetic BH3 organic peptides, or a derivative thereof, farnesyl-thiosalicylic acid (FTS), estradiol (E2), delta-tocotrienol, salinomycin, or curcumin, an antimetabolite, alkylator, corticosteroid, radiation, monoclonal antibody, platin or PARP inhibitor, epirubicin, cisplatin, dacarbazine, fludarabine/cyclophosphamide, dexamethasone, or doxorubicin, FTS, CMH, TMS, or estradiol (E2);
(iii) a cytostatic agent, cytocidal agent, actinomycin D, adriamycin, arsenic trioxide, asparaginase, bleomycin, busulfan, camptosar, carboplatinum, carmustine, chlorambucil, cisplatin, corticosteroids, colicheamicin, cyclophosphamide, daunorubicin, docetaxel, doxorubicin, epirubicin, etoposide, fludarabine, fluorouracil, gemcitabina, gemcitabine, gemzar, hydroxyurea, idarubicin, ifosfamide, irinotecan, lomustine, melphalan, mercaptomurine, methotrexate, mitomycin, mitoxantrone, oxaliplatin, paclitaxel, platinol, platinex, procarbizine, raltitrexeel, rixin, steroids, streptozocin, taxol, taxotere, thioguanine, thiotepa, tomudex, topotecan, treosulfan, trihydrate, vinblastine, vincristine, vindesine, vinorelbina, vinorelbine, duanomycin, dactinomysin, esorubisin, mafosfamide, cytosine arabinoside, bis-chloroethylnitrosurea, mitomycin C, mithramycin, prednisone, hydroxyprogesterone, testosterone, tamoxifen, dacarbazine, hexamethylmelamine, pentamethylmelamine, amsacrine, chlorambudil, methylcyclohexylnitrosurea, nitrogen mustards, cyclophosphamide, 6-mercaptopurine, 6-thioguanine, cytarabine, 5-azacytidine, deoxyco-formycin, 4-hydroxyperoxycyclophosphoramide, 5-fluorouracil (5-FU), 5-fluorodeoxyuridine (5-FUdR), colchicine, trimetrexate, teni-poside, diethylstilbestrol, a DNA damaging agent, nucleophosmin, an agent which induces cellular damage as part of an enhanced or synergistic process with another agent, a catalytic antibody, prodrugs, CHK1/2 inhibitor, CBP-501, AZD7762, histone deacetylase inhibitor, vorinostat, tumour necrosis factor related apoptosis inducing ligand, BH3 mimetic, ABT737, small molecule inhibitors, tyrosine kinase inhibitors, imatinib mesylate, gefitinib, erlotinib, monoclonal antibodies, rituximab, trastuzumab, a caspase activator, apoptin, survivin, an endocrine therapeutic, a biologic response modifier, interferon, interleukin, antibody, aptamer, siRNA, oligonucletoide, enzyme, ion channel and receptor inhibitor or activator, hyperthermia, cryotherapy, agent to attenuate any adverse effects, or antiemetic, an alkylating drug, mechlorethamine, chlorambucil, Cyclophosphamide, Melphalan, Ifosfamide, antimetabolite, Methotrexate, purine antagonist, pyrimidine antagonist, 6-Mercaptopurine, 5-Fluorouracil, Cytarabile, Gemcitabine, spindle poison, Vinblastine, Vincristine, Vinorelbine, Paclitaxel, podophyllotoxin, Etoposide, Irinotecan, Topotecan, antibiotic, doxorubicin, Bleomycin, Mitomycin, nitrosoureas, Carmustine, Lomustine, inorganic ion, Cisplatin, Carboplatin, enzyme, Asparaginase, hormone, Tamoxifen, Leuprolide, Flutamide, Megestrol, an agent that targets p53, p53 pathway members, IκB kinase, IKKβ, the proteasome/ubiquitin pathway, the 20S proteasome, the PI3K/Akt pathway, or mTOR, ONY-015, INGN201, PS1145, Bortezomib, CCI779, RAD-001, an siRNA targeting MDM2, mixed lineage kinase domain like (MLKL) protein, rapamycin (RAP) or derivatives and/or analogs thereof, everolimus or RAD001; CCI-779, ABT578, SAR543, ascomycin (an ethyl analog of FK506), AP23573, AP23841, KU-0063794, INK-128, EX2044, EX3855, EX7518, compounds that bind to the ATP-binding cleft of mTOR, AZD08055, OSIO27.

157. The method of claim 147, wherein
(i) said anti-CAA antibody and said second therapeutic agent exhibit enhanced therapeutic efficacy relative to said anti-CAA antibody and said second therapeutic agent administered individually;
(ii) said anti-CAA antibody comprises a NEO-201 antibody comprising:
(a) a light chain variable region polypeptide comprising the polypeptide of SEQ ID NO: 76 or a polypeptide comprising the CDRs thereof and having at least 90% identity thereto and/or a heavy chain variable region polypeptide comprising the polypeptide of SEQ ID NO: 81 or a polypeptide comprising the CDRs thereof and having at least 90% identity thereto;
(b) a light chain variable region polypeptide comprising the polypeptide of any one of SEQ ID NOs: 85-89 or a polypeptide comprising the CDRs thereof and having at least 90% identity thereto and optionally containing the light constant domain contained in SEQ ID NO: 52, 62, or 72 and/or a heavy chain variable region polypeptide comprising the polypeptide of any one of SEQ ID NOs: 90-94 or a polypeptide comprising the CDRs thereof and having at least 90% identity thereto and optionally containing the heavy constant domain contained in SEQ ID NO: 57, 67, or 74;
(c) a light chain comprising the polypeptide of SEQ ID NO: 96 and/or a heavy chain comprising the polypeptide of SEQ ID NO: 101, and said anti-CAA antibody and said second therapeutic agent are administered at a lower dosage than the effective dosage of said anti-CAA antibody or said second therapeutic agent when administered individually, wherein said method is for the treatment of a cancer that expresses an antigen specifically bound by said anti-CAA antibody;
(iii) said anti-CAA antibody is chimeric or humanized;
(iv) said anti-CAA antibody and said second therapeutic agent in said synergistic combination are administered to said subject together or separately;
(v) said synergistic combination results in apoptosis of cancer cells in said subject
(vi) said cancer is selected from the group consisting of: a carcinoma, colon cancer, pancreatic cancer, lung cancer, prostate cancer, melanoma, breast cancer, ovarian cancer, uterine cancer, cervical cancer, and mesothelioma, or is selected from the group consisting of: breast cancer, ovarian cancer, uterine cancer, cervical cancer;
(vii) said cancer comprises breast, ovarian, uterine, or cervical cancer;
(viii) the administration of said synergistic combination results in a synergistic effect on treatment outcome, wherein said synergistic effect is determined based on: reduction in the number of cancer cells or absence of the cancer cells; reduction in the tumor size; inhibition of cancer cell infiltration into peripheral organs including the spread of cancer into soft tissue and bone; inhibition of tumor metastasis; inhibition, to some extent, of tumor growth; relief of one or more of the symptoms associated with the specific cancer; reduced morbidity; reduced mortality; or improvement in quality of life.

158. The method of claim 148, wherein
(i) said anti-CAA antibody and said second therapeutic agent when administered exhibit enhanced therapeutic efficacy relative to said anti-CAA antibody and said second therapeutic agent administered individually;
(ii) said anti-CAA antibody comprises a NEO-201 antibody comprising:
(a) a light chain variable region polypeptide comprising the polypeptide of SEQ ID NO: 76 or a polypeptide comprising the CDRs thereof and having at least 90% identity thereto and/or a heavy chain variable region polypeptide comprising the polypeptide of SEQ ID NO: 81 or a polypeptide comprising the CDRs thereof and having at least 90% identity thereto;
(b) a light chain variable region polypeptide comprising the polypeptide of any one of SEQ ID NOs: 85-89 or a polypeptide comprising the CDRs thereof and having at least 90% identity thereto and optionally containing the light constant domain contained in SEQ ID NO: 52, 62, or 72 and/or a heavy chain variable region polypeptide comprising the polypeptide of any one of SEQ ID NOs: 90-94 or a polypeptide comprising the CDRs thereof and having at least 90% identity thereto and optionally containing the heavy constant domain contained in SEQ ID NO: 57, 67, or 74;
(c) a light chain comprising the polypeptide of SEQ ID NO: 96 and/or a heavy chain comprising the polypeptide of SEQ ID NO: 101, and said anti-CAA antibody and said second therapeutic agent are administered at a lower dosage than the effective dosage of said anti-CAA antibody or said second therapeutic agent when administered individually, wherein said method is for the treatment of a cancer that expresses an antigen specifically bound by said anti-CAA antibody;
(iii) said anti-CAA antibody is chimeric or humanized;
(iv) said anti-CAA antibody and said second therapeutic agent in said synergistic combination are administered to said subject together or separately;
(v) said synergistic combination results in apoptosis of cancer cells in said subject
(vi) said cancer is selected from the group consisting of: a carcinoma, colon cancer, pancreatic cancer, lung cancer, prostate cancer, melanoma, breast cancer, ovarian cancer, uterine cancer, cervical cancer, and mesothelioma, or is selected from the group consisting of: breast cancer, ovarian cancer, uterine cancer, cervical cancer;
(vii) said cancer comprises breast, ovarian, uterine, or cervical cancer;
(viii) the administration of said synergistic combination results in a synergistic effect on treatment outcome, wherein said synergistic effect is determined based on: reduction in the number of cancer cells or absence of the cancer cells; reduction in the tumor size; inhibition of cancer cell infiltration into peripheral organs including the spread of cancer into soft tissue and bone; inhibition of tumor metastasis; inhibition, to some extent, of tumor growth; relief of one or more of the symptoms associated with the specific cancer; reduced morbidity; reduced mortality; or improvement in quality of life.
